SEQUENCE LISTING (521 sequences)

SEQ ID NO: 1 - protein, 10 aa, Homo sapiens
  MISC_FEATURE (1)..(1): Xaa is Arg or is absent
XVWDLPGVLK

SEQ ID NO: 2 - protein, 10 aa, Homo sapiens
RVWDLPGVLK

SEQ ID NO: 3 - protein, 9 aa, Homo sapiens
VWDLPGVLK

SEQ ID NO: 4 - protein, 10 aa, Homo sapiens
  MISC_FEATURE (1)..(1): Xaa is Ala or Ser
XEIEQKIKEY

SEQ ID NO: 5 - protein, 10 aa, Homo sapiens
AEIEQKIKEY

SEQ ID NO: 6 - protein, 10 aa, Homo sapiens
SEIEQKIKEY

SEQ ID NO: 7 - protein, 10 aa, Homo sapiens
  MISC_FEATURE (8)..(8): Xaa is Pro or His
AAQTARQXPK

SEQ ID NO: 8 - protein, 10 aa, Homo sapiens
AAQTARQPPK

SEQ ID NO: 9 - protein, 10 aa, Homo sapiens
AAQTARQHPK

SEQ ID NO: 10 - protein, 9 aa, Homo sapiens
NESNTQKTY

SEQ ID NO: 11 - protein, 14 aa, Homo sapiens
QTDPRAGGGGGGDY

SEQ ID NO: 12 - protein, 9 aa, Homo sapiens
  MISC_FEATURE (2)..(2): Xaa is Glu or Ala
AXIQEKKEI

SEQ ID NO: 13 - protein, 9 aa, Homo sapiens
AEIQEKKEI

SEQ ID NO: 14 - protein, 9 aa, Homo sapiens
AAIQEKKEI

SEQ ID NO: 15 - protein, 9 aa, Homo sapiens
  MISC_FEATURE (5)..(5): Xaa is Ser or Ala
AELQXRLAA

SEQ ID NO: 16 - protein, 9 aa, Homo sapiens
AELQSRLAA

SEQ ID NO: 17 - protein, 9 aa, Homo sapiens
AELQARLAA

SEQ ID NO: 18 - protein, 10 aa, Homo sapiens
  MISC_FEATURE (7)..(7): Xaa is Ala or Val
APPAEKXVPV

SEQ ID NO: 19 - protein, 9 aa, Homo sapiens
APPAEKAPV

SEQ ID NO: 20 - protein, 9 aa, Homo sapiens
APPAEKVPV

SEQ ID NO: 21 - protein, 10 aa, Homo sapiens
  MISC_FEATURE (5)..(5): Xaa is Pro or Gln
APREXFAHSL

SEQ ID NO: 22 - protein, 10 aa, Homo sapiens
APREPFAHSL

SEQ ID NO: 23 - protein, 10 aa, Homo sapiens
APREQFAHSL

SEQ ID NO: 24 - protein, 9 aa, Homo sapiens
  MISC_FEATURE (5)..(5): Xaa is Ser or Asn
APREXAQAI

SEQ ID NO: 25 - protein, 9 aa, Homo sapiens
APRESAQAI

SEQ ID NO: 26 - protein, 9 aa, Homo sapiens
APRENAQAI

SEQ ID NO: 27 - protein, 9 aa, Homo sapiens
  MISC_FEATURE (9)..(9): Xaa is Phe or Ser
APRPFGSVX

SEQ ID NO: 28 - protein, 9 aa, Homo sapiens
APRPFGSVF

SEQ ID NO: 29 - protein, 9 aa, Homo sapiens
APRPFGSVS

SEQ ID NO: 30 - protein, 9 aa, Homo sapiens
  MISC_FEATURE (4)..(4): Xaa is Arg or Cys
APRXPPPPP

SEQ ID NO: 31 - protein, 9 aa, Homo sapiens
APRRPPPPP

SEQ ID NO: 32 - protein, 9 aa, Homo sapiens
APRCPPPPP

SEQ ID NO: 33 - protein, 9 aa, Homo sapiens
  MISC_FEATURE (7)..(7): Xaa is Phe or His
AQTARQXPK

SEQ ID NO: 34 - protein, 9 aa, Homo sapiens
AQTARQPPK

SEQ ID NO: 35 - protein, 9 aa, Homo sapiens
AQTARQHPK

SEQ ID NO: 36 - protein, 9 aa, Homo sapiens
  MISC_FEATURE (8)..(8): Xaa is Tyr or is absent
DRANRFEXL

SEQ ID NO: 37 - protein, 9 aa, Homo sapiens
DRANRFEYL

SEQ ID NO: 38 - protein, 8 aa, Homo sapiens
DRANRFEL

SEQ ID NO: 39 - protein, 8 aa, Homo sapiens
  MISC_FEATURE (7)..(7): Xaa is Lys, Arg, Met or Thr
DRFVARXL

SEQ ID NO: 40 - protein, 8 aa, Homo sapiens
DRFVARKL

SEQ ID NO: 41 - protein, 8 aa, Homo sapiens
DRFVARRL

SEQ ID NO: 42 - protein, 8 aa, Homo sapiens
DRFVARML

SEQ ID NO: 43 - protein, 8 aa, Homo sapiens
DRFVARTL

SEQ ID NO: 44 - protein, 9 aa, Homo sapiens
  MISC_FEATURE (2)..(2): Xaa is Glu or Gly
EXRGENTSY

SEQ ID NO: 45 - protein, 9 aa, Homo sapiens
EERGENTSY

SEQ ID NO: 46 - protein, 9 aa, Homo sapiens
EGRGENTSY

SEQ ID NO: 47 - protein, 10 aa, Homo sapiens
  MISC_FEATURE (6)..(6): Xaa is Asn or His
EEADGXKQWW

SEQ ID NO: 48 - protein, 10 aa, Homo sapiens
EEADGNKQWW

SEQ ID NO: 49 - protein, 10 aa, Homo sapiens
EEADGHKQWW

SEQ ID NO: 50 - protein, 9 aa, Homo sapiens
  MISC_FEATURE (7)..(7): Xaa is His or Gln
  misc_feature (8)..(8): Xaa can be any naturally occurring amino acid
EEALGLYXW

SEQ ID NO: 51 - protein, 9 aa, Homo sapiens
EEALGLYHW

SEQ ID NO: 52 - protein, 9 aa, Homo sapiens
EEALGLYQW

SEQ ID NO: 53 - protein, 9 aa, Homo sapiens
  MISC_FEATURE (7)..(7): Xaa is Arg or Ile
EEINLQXNI

SEQ ID NO: 54 - protein, 9 aa, Homo sapiens
EEINLQRNI

SEQ ID NO: 55 - protein, 9 aa, Homo sapiens
EEINLQINI

SEQ ID NO: 56 - protein, 9 aa, Homo sapiens
  MISC_FEATURE (5)..(5): Xaa is Val or Ile
EEIPXSSHY

SEQ ID NO: 57 - protein, 9 aa, Homo sapiens
EEIPVSSHY

SEQ ID NO: 58 - protein, 9 aa, Homo sapiens
EEIPISSHY

SEQ ID NO: 59 - protein, 10 aa, Homo sapiens
  MISC_FEATURE (5)..(5): Xaa is Val or Ile
EEIPXSSHYF

SEQ ID NO: 60 - protein, 10 aa, Homo sapiens
EEIPVSSHYF

SEQ ID NO: 61 - protein, 10 aa, Homo sapiens
EEIPISSHYF

SEQ ID NO: 62 - protein, 9 aa, Homo sapiens
  MISC_FEATURE (7)..(7): Xaa is Gly or Ser
EELLAVXKF

SEQ ID NO: 63 - protein, 9 aa, Homo sapiens
EELLAVGKF

SEQ ID NO: 64 - protein, 9 aa, Homo sapiens
EELLAVSKF

SEQ ID NO: 65 - protein, 10 aa, Homo sapiens
  MISC_FEATURE (7)..(7): Xaa is Glu or Lys
EESAVPXRSW

SEQ ID NO: 66 - protein, 10 aa, Homo sapiens
EESAVPERSW

SEQ ID NO: 67 - protein, 10 aa, Homo sapiens
EESAVPKRSW

SEQ ID NO: 68 - protein, 9 aa, Homo sapiens
  MISC_FEATURE (2)..(2): Xaa is Glu or Lys
EXEQSQSRW

SEQ ID NO: 69 - protein, 9 aa, Homo sapiens
EEEQSQSRW

SEQ ID NO: 70 - protein, 9 aa, Homo sapiens
EKEQSQSRW

SEQ ID NO: 71 - protein, 9 aa, Homo sapiens
  MISC_FEATURE (4)..(4): Xaa is Ala or Ser
ELQXRLAAL

SEQ ID NO: 72 - protein, 9 aa, Homo sapiens
ELQARLAAL

SEQ ID NO: 73 - protein, 9 aa, Homo sapiens
ELQSRLAAL

SEQ ID NO: 74 - protein, 12 aa, Homo sapiens
  MISC_FEATURE (5)..(5): Xaa is Ser or Phe
EPQGXGRQGNSL

SEQ ID NO: 75 - protein, 12 aa, Homo sapiens
EPQGSGRQGNSL

SEQ ID NO: 76 - protein, 12 aa, Homo sapiens
EPQGFGRQGNSL

SEQ ID NO: 77 - protein, 8 aa, Homo sapiens
  MISC_FEATURE (5)..(5): Xaa is Arg or Cys
ESKIXVLL

SEQ ID NO: 78 - protein, 8 aa, Homo sapiens
ESKIRVLL

SEQ ID NO: 79 - protein, 8 aa, Homo sapiens
ESKICVLL

SEQ ID NO: 80 - protein, 10 aa, Homo sapiens
  MISC_FEATURE (1)..(1): Xaa is Gly or Asp
XPRPSPTRSV

SEQ ID NO: 81 - protein, 10 aa, Homo sapiens
GPRPSPTRSV

SEQ ID NO: 82 - protein, 10 aa, Homo sapiens
DPRPSPTRSV

SEQ ID NO: 83 - protein, 9 aa, Homo sapiens
  MISC_FEATURE (3)..(3): Xaa is Asp or Gly
GEXKGIKAL

SEQ ID NO: 84 - protein, 9 aa, Homo sapiens
GEDKGIKAL

SEQ ID NO: 85 - protein, 9 aa, Homo sapiens
GEGKGIKAL

SEQ ID NO: 86 - protein, 9 aa, Homo sapiens
  MISC_FEATURE (3)..(3): Xaa is Ala or Glu
GRXGIVARL

SEQ ID NO: 87 - protein, 9 aa, Homo sapiens
GRAGIVARL

SEQ ID NO: 88 - protein, 9 aa, Homo sapiens
GREGIVARL

SEQ ID NO: 89 - protein, 14 aa, Homo sapiens
  MISC_FEATURE (12)..(12): Xaa is Ile or Val
GTLSPSLGNSSXLK

SEQ ID NO: 90 - protein, 14 aa, Homo sapiens
GTLSPSLGNSSILK

SEQ ID NO: 91 - protein, 14 aa, Homo sapiens
GTLSPSLGNSSVLK

SEQ ID NO: 92 - protein, 9 aa, Homo sapiens
  MISC_FEATURE (9)..(9): Xaa is Leu or Ile
HRVYLVRKX

SEQ ID NO: 93 - protein, 9 aa, Homo sapiens
HRVYLVRKL

SEQ ID NO: 94 - protein, 9 aa, Homo sapiens
HRVYLVRKI

SEQ ID NO: 95 - protein, 9 aa, Homo sapiens
  MISC_FEATURE (5)..(5): Xaa is Val or Leu
IYPQXLHSL

SEQ ID NO: 96 - protein, 9 aa, Homo sapiens
IYPQVLHSL

SEQ ID NO: 97 - protein, 9 aa, Homo sapiens
IYPQLLHSL

SEQ ID NO: 98 - protein, 10 aa, Homo sapiens
  MISC_FEATURE (6)..(6): Xaa is Asp or Gly
KEFEDXIINW

SEQ ID NO: 99 - protein, 10 aa, Homo sapiens
KEFEDDIINW

SEQ ID NO: 100 - protein, 10 aa, Homo sapiens
KEFEDGIINW

SEQ ID NO: 101 - protein, 10 aa, Homo sapiens
  MISC_FEATURE (8)..(8): Xaa is Asn or Ser
KEINEKSXIL

SEQ ID NO: 102 - protein, 10 aa, Homo sapiens
KEINEKSNIL

SEQ ID NO: 103 - protein, 10 aa, Homo sapiens
KEINEKSSIL

SEQ ID NO: 104 - protein, 9 aa, Homo sapiens
  MISC_FEATURE (6)..(6): Xaa is Ala or Gly
KLYSEXKTK

SEQ ID NO: 105 - protein, 9 aa, Homo sapiens
KLYSEAKTK

SEQ ID NO: 106 - protein, 9 aa, Homo sapiens
KLYSEGKTK

SEQ ID NO: 107 - protein, 9 aa, Homo sapiens
  MISC_FEATURE (9)..(9): Xaa is Arg, Trp or Gly
KRVGASYEX

SEQ ID NO: 108 - protein, 9 aa, Homo sapiens
KRVGASYER

SEQ ID NO: 109 - protein, 9 aa, Homo sapiens
KRVGASYEW

SEQ ID NO: 110 - protein, 9 aa, Homo sapiens
KRVGASYEG

SEQ ID NO: 111 - protein, 11 aa, Homo sapiens
  MISC_FEATURE (10)..(10): Xaa is Met or Thr
KVKTSLNEQXY

SEQ ID NO: 112 - protein, 11 aa, Homo sapiens
KVKTSLNEQMY

SEQ ID NO: 113 - protein, 11 aa, Homo sapiens
KVKTSLNEQTY

SEQ ID NO: 114 - protein, 9 aa, Homo sapiens
  MISC_FEATURE (2)..(2): Xaa is Tyr or His
KXMTAVVKL

SEQ ID NO: 115 - protein, 9 aa, Homo sapiens
KYMTAVVKL

SEQ ID NO: 116 - protein, 9 aa, Homo sapiens
KHMTAVVKL

SEQ ID NO: 117 - protein, 10 aa, Homo sapiens
  MISC_FEATURE (2)..(2): Xaa is Tyr or His
KXMTAVVKLF

SEQ ID NO: 118 - protein, 10 aa, Homo sapiens
KYMTAVVKLF

SEQ ID NO: 119 - protein, 10 aa, Homo sapiens
KHMTAVVKLF

SEQ ID NO: 120 - protein, 8 aa, Homo sapiens
  MISC_FEATURE (6)..(6): Xaa is His or Arg
LENGAXAY

SEQ ID NO: 121 - protein, 8 aa, Homo sapiens
LENGAHAY

SEQ ID NO: 122 - protein, 8 aa, Homo sapiens
LENGARAY

SEQ ID NO: 123 - protein, 9 aa, Homo sapiens
  MISC_FEATURE (5)..(5): Xaa is Cys or Arg
LPRVXGTTL

SEQ ID NO: 124 - protein, 9 aa, Homo sapiens
LPRVCGTTL

SEQ ID NO: 125 - protein, 9 aa, Homo sapiens
LPRVRGTTL

SEQ ID NO: 126 - protein, 8 aa, Homo sapiens
  MISC_FEATURE (8)..(8): Xaa is Leu or Ile
LPSKRVSX

SEQ ID NO: 127 - protein, 8 aa, Homo sapiens
LPSKRVSL

SEQ ID NO: 128 - protein, 8 aa, Homo sapiens
LPSKRVSI

SEQ ID NO: 129 - protein, 9 aa, Homo sapiens
  MISC_FEATURE (4)..(4): Xaa is Q or H
  MISC_FEATURE (4)..(4): Xaa is Gln or His
LRIXQREQL

SEQ ID NO: 130 - protein, 9 aa, Homo sapiens
LRIQQREQL

SEQ ID NO: 131 - protein, 9 aa, Homo sapiens
LRIHQREQL

SEQ ID NO: 132 - protein, 10 aa, Homo sapiens
  MISC_FEATURE (8)..(8): Xaa is Thr or Ile
MPSHLRNXLL

SEQ ID NO: 133 - protein, 10 aa, Homo sapiens
MPSHLRNTLL

SEQ ID NO: 134 - protein, 10 aa, Homo sapiens
MPSHLRNILL

SEQ ID NO: 135 - protein, 11 aa, Homo sapiens
  MISC_FEATURE (8)..(8): Xaa is Thr or Ile
MPSHLRNXLLM

SEQ ID NO: 136 - protein, 11 aa, Homo sapiens
MPSHLRNTLLM

SEQ ID NO: 137 - protein, 11 aa, Homo sapiens
MPSHLRNILLM

SEQ ID NO: 138 - protein, 9 aa, Homo sapiens
  MISC_FEATURE (8)..(8): Xaa is Arg or Lys
NSEEHSAXY

SEQ ID NO: 139 - protein, 9 aa, Homo sapiens
NSEEHSARY

SEQ ID NO: 140 - protein, 9 aa, Homo sapiens
NSEEHSAKY

SEQ ID NO: 141 - protein, 11 aa, Homo sapiens
  MISC_FEATURE (2)..(2): Xaa is His or Pro
PXRYRPGTVAL

SEQ ID NO: 142 - protein, 11 aa, Homo sapiens
PHRYRPGTVAL

SEQ ID NO: 143 - protein, 11 aa, Homo sapiens
PPRYRPGTVAL

SEQ ID NO: 144 - protein, 10 aa, Homo sapiens
  MISC_FEATURE (9)..(9): Xaa is Pro or Leu
PPSGLRLLXL

SEQ ID NO: 145 - protein, 10 aa, Homo sapiens
PPSGLRLLPL

SEQ ID NO: 146 - protein, 10 aa, Homo sapiens
PPSGLRLLLL

SEQ ID NO: 147 - protein, 9 aa, Homo sapiens
  MISC_FEATURE (2)..(2): Xaa is Glu or Asp
QXLIGKKEY

SEQ ID NO: 148 - protein, 9 aa, Homo sapiens
QELIGKKEY

SEQ ID NO: 149 - protein, 9 aa, Homo sapiens
QDLIGKKEY

SEQ ID NO: 150 - protein, 9 aa, Homo sapiens
  MISC_FEATURE (3)..(3): Xaa is Asn or Asp
  MISC_FEATURE (5)..(5): Xaa is Gln or His
QEXIXNLQL

SEQ ID NO: 151 - protein, 9 aa, Homo sapiens
QENIQNLQL

SEQ ID NO: 152 - protein, 9 aa, Homo sapiens
QENIHNLQL

SEQ ID NO: 153 - protein, 9 aa, Homo sapiens
QEDIQNLQL

SEQ ID NO: 154 - protein, 9 aa, Homo sapiens
QEDIHNLQL

SEQ ID NO: 155 - protein, 10 aa, Homo sapiens
  MISC_FEATURE (5)..(5): Xaa is Gly or Arg
QELDXVFQKL

SEQ ID NO: 156 - protein, 10 aa, Homo sapiens
QELDGVFQKL

SEQ ID NO: 157 - protein, 10 aa, Homo sapiens
QELDRVFQKL

SEQ ID NO: 158 - protein, 9 aa, Homo sapiens
  MISC_FEATURE (7)..(7): Xaa is Arg or Gly
QENQDPXRW

SEQ ID NO: 159 - protein, 9 aa, Homo sapiens
QENQDPRRW

SEQ ID NO: 160 - protein, 9 aa, Homo sapiens
QENQDPGRW

SEQ ID NO: 161 - protein, 8 aa, Homo sapiens
  MISC_FEATURE (8)..(8): Xaa is Tyr or Asn
QERSFQEX

SEQ ID NO: 162 - protein, 8 aa, Homo sapiens
QERSFQEY

SEQ ID NO: 163 - protein, 8 aa, Homo sapiens
QERSFQEN

SEQ ID NO: 164 - protein, 9 aa, Homo sapiens
  MISC_FEATURE (1)..(1): Xaa is Arg or Gly
XIFASRLYY

SEQ ID NO: 165 - protein, 9 aa, Homo sapiens
RIFASRLYY

SEQ ID NO: 166 - protein, 9 aa, Homo sapiens
GIFASRLYY

SEQ ID NO: 167 - protein, 9 aa, Homo sapiens
  MISC_FEATURE (7)..(7): Xaa is Thr, Ala or Asn
RANLRAXKL

SEQ ID NO: 168 - protein, 9 aa, Homo sapiens
RANLRATKL

SEQ ID NO: 169 - protein, 9 aa, Homo sapiens
RANLRAAKL

SEQ ID NO: 170 - protein, 9 aa, Homo sapiens
RANLRANKL

SEQ ID NO: 171 - protein, 9 aa, Homo sapiens
  MISC_FEATURE (4)..(4): Xaa is Gly or Glu
RPPXGSGPL

SEQ ID NO: 172 - protein, 9 aa, Homo sapiens
RPPGGSGPL

SEQ ID NO: 173 - protein, 9 aa, Homo sapiens
RPPEGSGPL

SEQ ID NO: 174 - protein, 10 aa, Homo sapiens
  MISC_FEATURE (4)..(4): Xaa is Gly or Glu
  MISC_FEATURE (10)..(10): Xaa is Leu, His, Arg or Pro
RPPXGSGPLX

SEQ ID NO: 175 - protein, 10 aa, Homo sapiens
RPPGGSGPLL

SEQ ID NO: 176 - protein, 10 aa, Homo sapiens
RPPGGSGPLH

SEQ ID NO: 177 - protein, 10 aa, Homo sapiens
RPPGGSGPLR

SEQ ID NO: 178 - protein, 10 aa, Homo sapiens
RPPGGSGPLP

SEQ ID NO: 179 - protein, 10 aa, Homo sapiens
RPPEGSGPLL

SEQ ID NO: 180 - protein, 10 aa, Homo sapiens
RPPEGSGPLH

SEQ ID NO: 181 - protein, 10 aa, Homo sapiens
RPPEGSGPLR

SEQ ID NO: 182 - protein, 10 aa, Homo sapiens
RPPEGSGPLP

SEQ ID NO: 183 - protein, 9 aa, Homo sapiens
  MISC_FEATURE (5)..(5): Xaa is Pro or Ser
RPPPXPAWL

SEQ ID NO: 184 - protein, 9 aa, Homo sapiens
RPPPPPAWL

SEQ ID NO: 185 - protein, 9 aa, Homo sapiens
RPPPSPAWL

SEQ ID NO: 186 - protein, 9 aa, Homo sapiens
  MISC_FEATURE (5)..(5): Xaa is Val or Ile
RREDXVLGR

SEQ ID NO: 187 - protein, 9 aa, Homo sapiens
RREDVVLGR

SEQ ID NO: 188 - protein, 9 aa, Homo sapiens
RREDIVLGR

SEQ ID NO: 189 - protein, 11 aa, Homo sapiens
  MISC_FEATURE (3)..(3): Xaa is Ala or Thr
RTXDNFDDILK

SEQ ID NO: 190 - protein, 11 aa, Homo sapiens
RTADNFDDILK

SEQ ID NO: 191 - protein, 11 aa, Homo sapiens
RTTDNFDDILK

SEQ ID NO: 192 - protein, 9 aa, Homo sapiens
  MISC_FEATURE (1)..(1): Xaa is Ser or Thr
XVLKPGNSK

SEQ ID NO: 193 - protein, 9 aa, Homo sapiens
SVLKPGNSK

SEQ ID NO: 194 - protein, 9 aa, Homo sapiens
TVLKPGNSK

SEQ ID NO: 195 - protein, 11 aa, Homo sapiens
  MISC_FEATURE (8)..(8): Xaa is Glu or Lys
SEESAVPXRSW

SEQ ID NO: 196 - protein, 11 aa, Homo sapiens
SEESAVPERSW

SEQ ID NO: 197 - protein, 11 aa, Homo sapiens
SEESAVPKRSW

SEQ ID NO: 198 - protein, 9 aa, Homo sapiens
  MISC_FEATURE (6)..(6): Xaa is Arg or Cys
SESKIXVLL

SEQ ID NO: 199 - protein, 9 aa, Homo sapiens
SESKIRVLL

SEQ ID NO: 200 - protein, 9 aa, Homo sapiens
SESKICVLL

SEQ ID NO: 201 - protein, 9 aa, Homo sapiens
  MISC_FEATURE (3)..(3): Xaa is Asp or Glu
SPXSSTPKL

SEQ ID NO: 202 - protein, 9 aa, Homo sapiens
SPDSSTPKL

SEQ ID NO: 203 - protein, 9 aa, Homo sapiens
SPESSTPKL

SEQ ID NO: 204 - protein, 10 aa, Homo sapiens
  MISC_FEATURE (5)..(5): Xaa is Asn or Lys
SPRGXLPLLL

SEQ ID NO: 205 - protein, 10 aa, Homo sapiens
SPRGNLPLLL

SEQ ID NO: 206 - protein, 10 aa, Homo sapiens
SPRGKLPLLL

SEQ ID NO: 207 - protein, 9 aa, Homo sapiens
  MISC_FEATURE (3)..(3): Xaa is Ala or Ser
SQXEIEQKI

SEQ ID NO: 208 - protein, 9 aa, Homo sapiens
SQAEIEQKI

SEQ ID NO: 209 - protein, 9 aa, Homo sapiens
SQSEIEQKI

SEQ ID NO: 210 - protein, 9 aa, Homo sapiens
  MISC_FEATURE (6)..(6): Xaa is Asn or Lys
SRVLQXVAF

SEQ ID NO: 211 - protein, 9 aa, Homo sapiens
SRVLQNVAF

SEQ ID NO: 212 - protein, 9 aa, Homo sapiens
SRVLQKVAF

SEQ ID NO: 213 - protein, 9 aa, Homo sapiens
  MISC_FEATURE (7)..(7): Xaa is Thr or Asn
SVSKLSXPK

SEQ ID NO: 214 - protein, 9 aa, Homo sapiens
SVSKLSTPK

SEQ ID NO: 215 - protein, 9 aa, Homo sapiens
SVSKLSNPK

SEQ ID NO: 216 - protein, 9 aa, Homo sapiens
  MISC_FEATURE (1)..(1): Xaa is Thr or Pro
XARPQSSAL

SEQ ID NO: 217 - protein, 9 aa, Homo sapiens
TARPQSSAL

SEQ ID NO: 218 - protein, 9 aa, Homo sapiens
PARPQSSAL

SEQ ID NO: 219 - protein, 9 aa, Homo sapiens
  MISC_FEATURE (9)..(9): Xaa is Ala or Val
TAKQKLDPX

SEQ ID NO: 220 - protein, 9 aa, Homo sapiens
TAKQKLDPA

SEQ ID NO: 221 - protein, 9 aa, Homo sapiens
TAKQKLDPV

SEQ ID NO: 222 - protein, 9 aa, Homo sapiens
  MISC_FEATURE (3)..(3): Xaa is Asn or Ser
TLXERFTSY

SEQ ID NO: 223 - protein, 9 aa, Homo sapiens
TLNERFTSY

SEQ ID NO: 224 - protein, 9 aa, Homo sapiens
TLSERFTSY

SEQ ID NO: 225 - protein, 11 aa, Homo sapiens
  MISC_FEATURE (11)..(11): Xaa is Leu or Val
TPRNTYKMTSX

SEQ ID NO: 226 - protein, 11 aa, Homo sapiens
TPRNTYKMTSL

SEQ ID NO: 227 - protein, 11 aa, Homo sapiens
TPRNTYKMTSV

SEQ ID NO: 228 - protein, 9 aa, Homo sapiens
  MISC_FEATURE (9)..(9): Xaa is Leu or Pro
TPRPIQSSX

SEQ ID NO: 229 - protein, 9 aa, Homo sapiens
TPRPIQSSL

SEQ ID NO: 230 - protein, 9 aa, Homo sapiens
TPRPIQSSP

SEQ ID NO: 231 - protein, 8 aa, Homo sapiens
  MISC_FEATURE (6)..(6): Xaa is Arg or Ser
TPVDDXSL

SEQ ID NO: 232 - protein, 8 aa, Homo sapiens
TPVDDRSL

SEQ ID NO: 233 - protein, 8 aa, Homo sapiens
TPVDDSSL

SEQ ID NO: 234 - protein, 9 aa, Homo sapiens
  MISC_FEATURE (3)..(3): Xaa is Arg or Ser
TQXPADVIF

SEQ ID NO: 235 - protein, 9 aa, Homo sapiens
TQRPADVIF

SEQ ID NO: 236 - protein, 9 aa, Homo sapiens
TQSPADVIF

SEQ ID NO: 237 - protein, 9 aa, Homo sapiens
  MISC_FEATURE (3)..(3): Xaa is Tyr or Cys
TVXHSPVSR

SEQ ID NO: 238 - protein, 9 aa, Homo sapiens
TVYHSPVSR

SEQ ID NO: 239 - protein, 9 aa, Homo sapiens
TVCHSPVSR

SEQ ID NO: 240 - protein, 10 aa, Homo sapiens
  MISC_FEATURE (7)..(7): Xaa is Asn or His
VEEADGXKQW

SEQ ID NO: 241 - protein, 10 aa, Homo sapiens
VEEADGNKQW

SEQ ID NO: 242 - protein, 10 aa, Homo sapiens
VEEADGHKQW

SEQ ID NO: 243 - protein, 10 aa, Homo sapiens
  MISC_FEATURE (8)..(8): Xaa is His or Arg
VYNNIMRXYL

SEQ ID NO: 244 - protein, 10 aa, Homo sapiens
VYNNIMRHYL

SEQ ID NO: 245 - protein, 10 aa, Homo sapiens
VYNNIMRRYL

SEQ ID NO: 246 - protein, 9 aa, Homo sapiens
  MISC_FEATURE (6)..(6): Xaa is Ser or Arg
YPRAGXKPP

SEQ ID NO: 247 - protein, 9 aa, Homo sapiens
YPRAGSKPP

SEQ ID NO: 248 - protein, 9 aa, Homo sapiens
YPRAGRKPP

SEQ ID NO: 249 - protein, 10 aa, Homo sapiens
  MISC_FEATURE (7)..(7): Xaa is Ile or Val
YTDSSSXLNY

SEQ ID NO: 250 - protein, 10 aa, Homo sapiens
YTDSSSILNY

SEQ ID NO: 251 - protein, 10 aa, Homo sapiens
YTDSSSVLNY

SEQ ID NO: 252 - protein, 10 aa, Homo sapiens
  MISC_FEATURE (9)..(9): Xaa is Ile or Met
QELEEKLNXL

SEQ ID NO: 253 - protein, 9 aa, Homo sapiens
  MISC_FEATURE (3)..(3): Xaa is Val or Ala
REXLELDSI

SEQ ID NO: 254 - protein, 9 aa, Homo sapiens
  MISC_FEATURE (1)..(1): Xaa is Arg or Gln
XLAPTLSQL

SEQ ID NO: 255 - protein, 9 aa, Homo sapiens
  MISC_FEATURE (5)..(5): Xaa is Asp or Asn
QEFIXNPKW

SEQ ID NO: 256 - protein, 11 aa, Homo sapiens
  MISC_FEATURE (5)..(5): Xaa is Gly or Ala
AEELXGPVHAL

SEQ ID NO: 257 - protein, 9 aa, Homo sapiens
  MISC_FEATURE (9)..(9): Xaa is Ala or Gly
SESEDRLVX

SEQ ID NO: 258 - protein, 9 aa, Homo sapiens
  MISC_FEATURE (9)..(9): Xaa is Leu or Phe
ILSEVERNX

SEQ ID NO: 259 - protein, 13 aa, Homo sapiens
  MISC_FEATURE (10)..(10): Xaa is Ile or Val
EENGRKEIDXKKY

SEQ ID NO: 260 - protein, 9 aa, Homo sapiens
  MISC_FEATURE (6)..(6): Xaa is Arg or Lys
QEEQTXVAL

SEQ ID NO: 261 - protein, 10 aa, Homo sapiens
  MISC_FEATURE (1)..(1): Xaa is Ile or Ser
XLAPCKLETV

SEQ ID NO: 262 - protein, 11 aa, Homo sapiens
  MISC_FEATURE (8)..(8): Xaa is Thr or Ile
RSVDVTNXTFL

SEQ ID NO: 263 - protein, 10 aa, Homo sapiens
  MISC_FEATURE (6)..(6): Xaa is Asn or His
EEADGXKQWW

SEQ ID NO: 264 - protein, 9 aa, Homo sapiens
  MISC_FEATURE (9)..(9): Xaa is Ala or Thr
AEVEHVVNX

SEQ ID NO: 265 - protein, 9 aa, Homo sapiens
  MISC_FEATURE (4)..(4): Xaa is Ala or Thr
KEIXKTVLI

SEQ ID NO: 266 - protein, 9 aa, Homo sapiens
  MISC_FEATURE (2)..(2): Xaa is Leu or Ile
KXRGVINQL

SEQ ID NO: 267 - protein, 8 aa, Homo sapiens
  MISC_FEATURE (5)..(5): Xaa is Glu or Gln
MLRSXLLL

SEQ ID NO: 268 - protein, 9 aa, Homo sapiens
  MISC_FEATURE (2)..(2): Xaa is Gln or Glu
RXPDLVLRL

SEQ ID NO: 269 - protein, 9 aa, Homo sapiens
  MISC_FEATURE (5)..(5): Xaa is Ala or Thr
LLLAXPAQA

SEQ ID NO: 270 - protein, 10 aa, Homo sapiens
  MISC_FEATURE (1)..(1): Xaa is Glu or Gln
XETAIYKGDY

SEQ ID NO: 271 - protein, 9 aa, Homo sapiens
  MISC_FEATURE (2)..(2): Xaa is Ile or Val
LXDTSRHYL

SEQ ID NO: 272 - protein, 9 aa, Homo sapiens
  MISC_FEATURE (8)..(8): Xaa is Arg or Cys
KILEKEIXV

SEQ ID NO: 273 - protein, 9 aa, Homo sapiens
VEVPEAHQL

SEQ ID NO: 274 - protein, 9 aa, Homo sapiens
  MISC_FEATURE (4)..(4): Xaa is Ile or Met
MESXNPHKY

SEQ ID NO: 275 - protein, 10 aa, Homo sapiens
  MISC_FEATURE (7)..(7): Xaa is Ile or Asn
QELETSXKKI

SEQ ID NO: 276 - protein, 10 aa, Homo sapiens
  MISC_FEATURE (1)..(1): Xaa is Asn or Asp
XEVLIHSSQY

SEQ ID NO: 277 - protein, 11 aa, Homo sapiens
  MISC_FEATURE (11)..(11): Xaa is Leu or Pro
SLLESSRSQEX

SEQ ID NO: 278 - protein, 9 aa, Homo sapiens
  MISC_FEATURE (9)..(9): Xaa is Val or Leu
ALSGHLETX

SEQ ID NO: 279 - protein, 10 aa, Homo sapiens
  MISC_FEATURE (4)..(4): Xaa is Gln or Lys
AELXGFHRSF

SEQ ID NO: 280 - protein, 9 aa, Homo sapiens
  MISC_FEATURE (7)..(7): Xaa is Ala or Pro
HLEEQIXKV

SEQ ID NO: 281 - protein, 10 aa, Homo sapiens
  MISC_FEATURE (1)..(1): Xaa is Thr or Ile
XLLEDGTFKV

SEQ ID NO: 282 - protein, 9 aa, Homo sapiens
  MISC_FEATURE (5)..(5): Xaa is Ile or Val
VIAEXLRGV

SEQ ID NO: 283 - protein, 9 aa, Homo sapiens
  MISC_FEATURE (3)..(3): Xaa is Ile or Val
AEXLRGVRL

SEQ ID NO: 284 - protein, 10 aa, Homo sapiens
  MISC_FEATURE (7)..(7): Xaa is Asp or Glu
KLAENIXAQL

SEQ ID NO: 285 - protein, 11 aa, Homo sapiens
  MISC_FEATURE (5)..(5): Xaa is Asp or Glu
AENIXAQLKRM

SEQ ID NO: 286 - protein, 9 aa, Homo sapiens
  MISC_FEATURE (8)..(8): Xaa is Ala or Thr
FLQAKQIXL

SEQ ID NO: 287 - protein, 9 aa, Homo sapiens
  MISC_FEATURE (6)..(6): Xaa is Thr, Ile or Arg
DEIVCXQHW

SEQ ID NO: 288 - protein, 9 aa, Homo sapiens
  MISC_FEATURE (7)..(7): Xaa is Phe or Cys
YTWEEVXRV

SEQ ID NO: 289 - protein, 9 aa, Homo sapiens
  MISC_FEATURE (8)..(8): Xaa is Leu, Met or Val
KTDKTLVXL

SEQ ID NO: 290 - protein, 9 aa, Homo sapiens
  MISC_FEATURE (9)..(9): Xaa is Ala or Pro
SQVQVPLEX

SEQ ID NO: 291 - protein, 9 aa, Homo sapiens
  MISC_FEATURE (8)..(8): Xaa is Arg or His
EEYEELLXY

SEQ ID NO: 292 - protein, 14 aa, Homo sapiens
  MISC_FEATURE (4)..(4): Xaa is Asp or Glu
TEGXALDALGLKRY

SEQ ID NO: 293 - protein, 9 aa, Homo sapiens
  MISC_FEATURE (2)..(2): Xaa is Gln or His
GXYTDLLRL

SEQ ID NO: 294 - protein, 9 aa, Homo sapiens
  MISC_FEATURE (2)..(2): Xaa is Glu or Lys
IXDRQYKDY

SEQ ID NO: 295 - protein, 9 aa, Homo sapiens
  MISC_FEATURE (7)..(7): Xaa is His or Arg
AENDFVXRI

SEQ ID NO: 296 - protein, 9 aa, Homo sapiens
  MISC_FEATURE (2)..(2): Xaa Leu or Val
RXLQEQHQL

SEQ ID NO: 297 - protein, 9 aa, Homo sapiens
  misc_feature (1)..(1): Xaa can be any naturally occurring amino acid
  MISC_FEATURE (2)..(2): Xaa is Leu or Ser
XLQEELEKL

SEQ ID NO: 298 - protein, 9 aa, Homo sapiens
  MISC_FEATURE (2)..(2): Xaa is Leu or Ser
GXSPLLQKI

SEQ ID NO: 299 - protein, 9 aa, Homo sapiens
  MISC_FEATURE (6)..(6): Xaa is Ser or Pro
TEMEIXRAA

SEQ ID NO: 300 - protein, 9 aa, Homo sapiens
  MISC_FEATURE (2)..(2): Xaa is Gln or Arg
EXQLLYRSW

SEQ ID NO: 301 - protein, 10 aa, Homo sapiens
  MISC_FEATURE (4)..(4): Xaa is Asp or Gly
TEVXEAGSQL

SEQ ID NO: 302 - protein, 11 aa, Homo sapiens
  MISC_FEATURE (1)..(1): Xaa is Gln or Glu
XEAPESATVIF

SEQ ID NO: 303 - protein, 9 aa, Homo sapiens
  MISC_FEATURE (5)..(5): Xaa is Glu or Asp
TETQXKNTL

SEQ ID NO: 304 - protein, 8 aa, Homo sapiens
  MISC_FEATURE (3)..(3): Xaa is Val or Ile
AEXRAENL

SEQ ID NO: 305 - protein, 9 aa, Homo sapiens
  MISC_FEATURE (8)..(8): Xaa is Ile or Thr
LLWAGPVXA

SEQ ID NO: 306 - protein, 9 aa, Homo sapiens
  MISC_FEATURE (3)..(3): Xaa is Asn or Asp
KEXQEAEKL

SEQ ID NO: 307 - protein, 9 aa, Homo sapiens
  MISC_FEATURE (1)..(1): Xaa is Gln or Arg
XEYQVKLQA

SEQ ID NO: 308 - protein, 9 aa, Homo sapiens
  MISC_FEATURE (1)..(1): Xaa is Leu, Met or Val
XEADLPRSW

SEQ ID NO: 309 - protein, 9 aa, Homo sapiens
  MISC_FEATURE (5)..(5): Xaa is Gly or Glu
IEATXFDRL

SEQ ID NO: 310 - protein, 9 aa, Homo sapiens
  MISC_FEATURE (2)..(2): Xaa is Leu or Pro
SXDDHVVAV

SEQ ID NO: 311 - protein, 9 aa, Homo sapiens
  MISC_FEATURE (7)..(7): Xaa is His or Arg
QEPFVFXEF

SEQ ID NO: 312 - protein, 9 aa, Homo sapiens
  MISC_FEATURE (7)..(7): Xaa is Leu or Ser
ELQEKFXSL

SEQ ID NO: 313 - protein, 9 aa, Homo sapiens
  MISC_FEATURE (8)..(8): Xaa is Pro or Ala
SLFFRKVXF

SEQ ID NO: 314 - protein, 11 aa, Homo sapiens
  MISC_FEATURE (9)..(9): Xaa is Arg or Trp
AMYDKGPFXSK

SEQ ID NO: 315 - protein, 9 aa, Homo sapiens
  MISC_FEATURE (9)..(9): Xaa is Gly or Arg
RVSLPTSPX

SEQ ID NO: 316 - protein, 9 aa, Homo sapiens
  MISC_FEATURE (9)..(9): Xaa is Lys or Asn
VMGNPGTFX

SEQ ID NO: 317 - protein, 9 aa, Homo sapiens
VLHDDLLEA

SEQ ID NO: 318 - protein, 10 aa, Homo sapiens
KECVLHDDLL

SEQ ID NO: 319 - protein, 9 aa, Homo sapiens
YIGEVLVSL

SEQ ID NO: 320 - protein, 10 aa, Homo sapiens
EEKRGSLHVW

SEQ ID NO: 321 - protein, 11 aa, Homo sapiens
EEKRGSLYVVV

SEQ ID NO: 322 - protein, 9 aa, Homo sapiens
TPNQRQNVC

SEQ ID NO: 323 - protein, 10 aa, Homo sapiens
RVWDLPGVLK

SEQ ID NO: 324 - protein, 11 aa, Homo sapiens
MEIFIEVFSHF

SEQ ID NO: 325 - protein, 15 aa, Homo sapiens
PEIWEGEPPCLPPRD

SEQ ID NO: 326 - protein, 9 aa, Homo sapiens
DYLQYVLQI

SEQ ID NO: 327 - protein, 9 aa, Homo sapiens
DYLQCVLQI

SEQ ID NO: 328 - protein, 10 aa, Homo sapiens
SLPRGTSTPK

SEQ ID NO: 329 - protein, 11 aa, Homo sapiens
SVAPALALFPA

SEQ ID NO: 330 - protein, 11 aa, Homo sapiens
RPHAIRRPLAL

SEQ ID NO: 331 - protein, 10 aa, Homo sapiens
WATLPLLCAR

SEQ ID NO: 332 - protein, 12 aa, Homo sapiens
SRSSSAELDRSR

SEQ ID NO: 333 - protein, 8 aa, Homo sapiens
LPHNHTDL

SEQ ID NO: 334 - protein, 9 aa, Homo sapiens
TIRYPDPVI

SEQ ID NO: 335 - protein, 9 aa, Homo sapiens
RTLDKVLEV

SEQ ID NO: 336 - protein, 9 aa, Homo sapiens
VTEPGTAQY

SEQ ID NO: 337 - protein, 10 aa, Homo sapiens
AELLNIPFLY

SEQ ID NO: 338 - protein, 9 aa, Homo sapiens
ATLPLLCAR

SEQ ID NO: 339 - protein, 10 aa, Homo sapiens
WATLPLLCAR

SEQ ID NO: 340 - protein, 11 aa, Homo sapiens
SPSVDKARAEL

SEQ ID NO: 341 - protein, 9 aa, Homo sapiens
FIDSYICQV

SEQ ID NO: 342 - protein, 9 aa, Homo sapiens
IVDCLTEMY

SEQ ID NO: 343 - protein, 10 aa, Homo sapiens
RESEEESVSL

SEQ ID NO: 344 - protein, 12 aa, Homo sapiens
HIENFSDIDMGE

SEQ ID NO: 345 - protein, 19 aa, Homo sapiens
GSTASKGRYIPPHLRNREA

SEQ ID NO: 346 - protein, 10 aa, Homo sapiens
VIKVNDTVQI

SEQ ID NO: 347 - protein, 11 aa, Homo sapiens
EVLLRPGLHFR

SEQ ID NO: 348 - protein, 10 aa, Homo sapiens
  MISC_FEATURE (8)..(8): Xaa is Ala or Val
APKKPTGXDL

SEQ ID NO: 349 - protein, 10 aa, Homo sapiens
APKKPTGADL

SEQ ID NO: 350 - protein, 10 aa, Homo sapiens
APKKPTGVDL

SEQ ID NO: 351 - protein, 9 aa, Homo sapiens
  MISC_FEATURE (9)..(9): Xaa is His or Tyr
ASELHTSLX

SEQ ID NO: 352 - protein, 9 aa, Homo sapiens
ASELHTSLH

SEQ ID NO: 353 - protein, 9 aa, Homo sapiens
ASELHTSLY

SEQ ID NO: 354 - protein, 9 aa, Homo sapiens
  MISC_FEATURE (3)..(3): Xaa is Val or Leu
EEXKLRQQL

SEQ ID NO: 355 - protein, 9 aa, Homo sapiens
EEVKLRQQL

SEQ ID NO: 356 - protein, 9 aa, Homo sapiens
EELKLRQQL

SEQ ID NO: 357 - protein, 11 aa, Homo sapiens
  MISC_FEATURE (2)..(2): Xaa is Leu or Ile
EXDPSNTKALY

SEQ ID NO: 358 - protein, 11 aa, Homo sapiens
ELDPSNTKALY

SEQ ID NO: 359 - protein, 11 aa, Homo sapiens
EIDPSNTKALY

SEQ ID NO: 360 - protein, 9 aa, Homo sapiens
  MISC_FEATURE (4)..(4): Xaa is Val or Glu
VPNXKSGAL

SEQ ID NO: 361 - protein, 9 aa, Homo sapiens
VPNVKSGAL

SEQ ID NO: 362 - protein, 9 aa, Homo sapiens
VPNEKSGAL

SEQ ID NO: 363 - protein, 10 aa, Homo sapiens
  MISC_FEATURE (2)..(2): Xaa is Ser or Pro
IXRAAAERSL

SEQ ID NO: 364 - protein, 10 aa, Homo sapiens
ISRAAAERSL

SEQ ID NO: 365 - protein, 10 aa, Homo sapiens
IPRAAAERSL

SEQ ID NO: 366 - protein, 9 aa, Homo sapiens
  MISC_FEATURE (8)..(8): Xaa is Pro, Thr or Ser
LPSDDRGXL

SEQ ID NO: 367 - protein, 9 aa, Homo sapiens
LPSDDRGPL

SEQ ID NO: 368 - protein, 9 aa, Homo sapiens
LPSDDRGSL

SEQ ID NO: 369 - protein, 9 aa, Homo sapiens
LPSDDRGTL

SEQ ID NO: 370 - protein, 11 aa, Homo sapiens
  MISC_FEATURE (2)..(2): Xaa is Cys or Ser
LXEKPTVTTVY

SEQ ID NO: 371 - protein, 11 aa, Homo sapiens
LCEKPTVTTVY

SEQ ID NO: 372 - protein, 11 aa, Homo sapiens
LSEKPTVTTVY

SEQ ID NO: 373 - protein, 12 aa, Homo sapiens
  MISC_FEATURE (8)..(8): Xaa is Ser or Asn
RPRAPREXAQAI

SEQ ID NO: 374 - protein, 12 aa, Homo sapiens
RPRAPRESAQAI

SEQ ID NO: 375 - protein, 12 aa, Homo sapiens
RPRAPRENAQAI

SEQ ID NO: 376 - protein, 9 aa, Homo sapiens
  MISC_FEATURE (1)..(1): Xaa is His or Arg
XESPIFKQF

SEQ ID NO: 377 - protein, 9 aa, Homo sapiens
HESPIFKQF

SEQ ID NO: 378 - protein, 9 aa, Homo sapiens
RESPIFKQF

SEQ ID NO: 379 - protein, 11 aa, Homo sapiens
  MISC_FEATURE (11)..(11): Xaa is Leu or Val
TPRNTYKMTSX

SEQ ID NO: 380 - protein, 11 aa, Homo sapiens
TPRNTYKMTSL

SEQ ID NO: 381 - protein, 11 aa, Homo sapiens
TPRNTYKMTSV

SEQ ID NO: 382 - protein, 9 aa, Homo sapiens
  MISC_FEATURE (6)..(6): Xaa is Ile or Val
VPREYXRAL

SEQ ID NO: 383 - protein, 9 aa, Homo sapiens
VPREYIRAL

SEQ ID NO: 384 - protein, 9 aa, Homo sapiens
VPREYVRAL

SEQ ID NO: 385 - protein, 10 aa, Homo sapiens
  MISC_FEATURE (8)..(8): Xaa is Ile or Val
RPRARYYXQV

SEQ ID NO: 386 - protein, 10 aa, Homo sapiens
RPRARYYIQV

SEQ ID NO: 387 - protein, 10 aa, Homo sapiens
RPRARYYVQV

SEQ ID NO: 388 - protein, 9 aa, Homo sapiens
  MISC_FEATURE (8)..(8): Xaa is Ser or Ala
SAFADRPXF

SEQ ID NO: 389 - protein, 9 aa, Homo sapiens
SAFADRPSF

SEQ ID NO: 390 - protein, 9 aa, Homo sapiens
SAFADRPAF

SEQ ID NO: 391 - protein, 9 aa, Homo sapiens
  MISC_FEATURE (1)..(1): Xaa is Val or Ala
XPEEARPAL

SEQ ID NO: 392 - protein, 9 aa, Homo sapiens
VPEEARPAL

SEQ ID NO: 393 - protein, 9 aa, Homo sapiens
APEEARPAL

SEQ ID NO: 394 - protein, 9 aa, Homo sapiens
  MISC_FEATURE (7)..(7): Xaa is Val or Met
NLDKNTXGY

SEQ ID NO: 395 - protein, 9 aa, Homo sapiens
NLDKNTVGY

SEQ ID NO: 396 - protein, 9 aa, Homo sapiens
NLDKNTMGY

SEQ ID NO: 397 - protein, 11 aa, Homo sapiens
  misc_feature (4)..(4): Xaa is Val or Ala
SPRXPVSPLKF

SEQ ID NO: 398 - protein, 11 aa, Homo sapiens
SPRVPVSPLKF

SEQ ID NO: 399 - protein, 11 aa, Homo sapiens
SPRAPVSPLKF

SEQ ID NO: 400 - protein, 13 aa, Homo sapiens
  MISC_FEATURE (2)..(2): Xaa is Leu or Pro
SXRPQGLSNPSTL

SEQ ID NO: 401 - protein, 13 aa, Homo sapiens
SLRPQGLSNPSTL

SEQ ID NO: 402 - protein, 13 aa, Homo sapiens
SPRPQGLSNPSTL

SEQ ID NO: 403 - protein, 9 aa, Homo sapiens
  MISC_FEATURE (9)..(9): Xaa is Pro or Leu
TPRPIQSSX

SEQ ID NO: 404 - protein, 9 aa, Homo sapiens
TPRPIQSSP

SEQ ID NO: 405 - protein, 9 aa, Homo sapiens
TPRPIQSSL

SEQ ID NO: 406 - protein, 9 aa, Homo sapiens
  MISC_FEATURE (3)..(3): Xaa is Arg or Pro
HPXQEQIAL

SEQ ID NO: 407 - protein, 9 aa, Homo sapiens
HPRQEQIAL

SEQ ID NO: 408 - protein, 9 aa, Homo sapiens
HPPQEQIAL

SEQ ID NO: 409 - protein, 9 aa, Homo sapiens
  MISC_FEATURE (7)..(7): Xaa is Thr, Ser or Ile
YYRTNHXVM

SEQ ID NO: 410 - protein, 9 aa, Homo sapiens
YYRTNHTVM

SEQ ID NO: 411 - protein, 9 aa, Homo sapiens
YYRTNHIVM

SEQ ID NO: 412 - protein, 9 aa, Homo sapiens
YYRTNHSVM

SEQ ID NO: 413 - protein, 11 aa, Homo sapiens
  MISC_FEATURE (9)..(9): Xaa is Arg, Thr or Lys
KEMDSDQQXSY

SEQ ID NO: 414 - protein, 11 aa, Homo sapiens
KEMDSDQQRSY

SEQ ID NO: 415 - protein, 11 aa, Homo sapiens
KEMDSDQQTSY

SEQ ID NO: 416 - protein, 11 aa, Homo sapiens
KEMDSDQQKSY

SEQ ID NO: 417 - protein, 9 aa, Homo sapiens
  MISC_FEATURE (1)..(1): Xaa is Met, Leu or Val
XELQQKAEF

SEQ ID NO: 418 - protein, 9 aa, Homo sapiens
MELQQKAEF

SEQ ID NO: 419 - protein, 9 aa, Homo sapiens
LELQQKAEF

SEQ ID NO: 420 - protein, 9 aa, Homo sapiens
VELQQKAEF

SEQ ID NO: 421 - protein, 9 aa, Homo sapiens
  MISC_FEATURE (1)..(1): Xaa is Ser or Tyr
XGGPLRSEY

SEQ ID NO: 422 - protein, 9 aa, Homo sapiens
SGGPLRSEY

SEQ ID NO: 423 - protein, 9 aa, Homo sapiens
YGGPLRSEY

SEQ ID NO: 424 - protein, 9 aa, Homo sapiens
  MISC_FEATURE (4)..(4): Xaa is Gly or Ala
TEAXVQKQW

SEQ ID NO: 425 - protein, 9 aa, Homo sapiens
TEAGVQKQW

SEQ ID NO: 426 - protein, 9 aa, Homo sapiens
TEAAVQKQW

SEQ ID NO: 427 - protein, 9 aa, Homo sapiens
  MISC_FEATURE (3)..(3): Xaa is Arg or His
RPXPEDQRL

SEQ ID NO: 428 - protein, 9 aa, Homo sapiens
RPRPEDQRL

SEQ ID NO: 429 - protein, 9 aa, Homo sapiens
RPHPEDQRL

SEQ ID NO: 430 - protein, 14 aa, Homo sapiens
  MISC_FEATURE (6)..(6): Xaa is Gln or Lys
LPRGMXPTEFFQSL

SEQ ID NO: 431 - protein, 14 aa, Homo sapiens
LPRGMQPTEFFQSL

SEQ ID NO: 432 - protein, 14 aa, Homo sapiens
LPRGMKPTEFFQSL

SEQ ID NO: 433 - protein, 9 aa, Homo sapiens
  MISC_FEATURE (5)..(5): Xaa is Ala or Val
LARPXSAAL

SEQ ID NO: 434 - protein, 9 aa, Homo sapiens
LARPASAAL

SEQ ID NO: 435 - protein, 9 aa, Homo sapiens
LARPVSAAL

SEQ ID NO: 436 - protein, 9 aa, Homo sapiens
  MISC_FEATURE (5)..(5): Xaa is Ser or Asn
APREXAQAI

SEQ ID NO: 437 - protein, 9 aa, Homo sapiens
APRESAQAI

SEQ ID NO: 438 - protein, 9 aa, Homo sapiens
APRENAQAI

SEQ ID NO: 439 - protein, 12 aa, Homo sapiens
  MISC_FEATURE (1)..(1): Xaa is Arg or Gln
XPRAPRESAQAI

SEQ ID NO: 440 - protein, 12 aa, Homo sapiens
RPRAPRESAQAI

SEQ ID NO: 441 - protein, 12 aa, Homo sapiens
QPRAPRESAQAI

SEQ ID NO: 442 - protein, 10 aa, Homo sapiens
  MISC_FEATURE (2)..(2): Xaa is Pro or Leu
RXRKEVKEEL

SEQ ID NO: 443 - protein, 10 aa, Homo sapiens
RPRKEVKEEL

SEQ ID NO: 444 - protein, 10 aa, Homo sapiens
RLRKEVKEEL

SEQ ID NO: 445 - protein, 10 aa, Homo sapiens
  MISC_FEATURE (2)..(2): Xaa is Pro or Leu
SXYPRVKVDF

SEQ ID NO: 446 - protein, 10 aa, Homo sapiens
SPYPRVKVDF

SEQ ID NO: 447 - protein, 10 aa, Homo sapiens
SLYPRVKVDF

SEQ ID NO: 448 - protein, 9 aa, Homo sapiens
  MISC_FEATURE (3)..(3): Xaa is Phe or Leu
IPXSNPRVL

SEQ ID NO: 449 - protein, 9 aa, Homo sapiens
IPFSNPRVL

SEQ ID NO: 450 - protein, 9 aa, Homo sapiens
IPLSNPRVL

SEQ ID NO: 451 - protein, 13 aa, Homo sapiens
  MISC_FEATURE (4)..(4): Xaa is Ser, Thr or Ala
  misc_feature (5)..(5): Xaa can be any naturally occurring amino acid
EEVTXSEDKRKTY

SEQ ID NO: 452 - protein, 13 aa, Homo sapiens
EEVTSSEDKRKTY

SEQ ID NO: 453 - protein, 13 aa, Homo sapiens
EEVTTSEDKRKTY

SEQ ID NO: 454 - protein, 13 aa, Homo sapiens
EEVTASEDKRKTY

SEQ ID NO: 455 - protein, 9 aa, Homo sapiens
  MISC_FEATURE (7)..(7): Xaa is Ile or Val
FSEPRAXFY

SEQ ID NO: 456 - protein, 9 aa, Homo sapiens
FSEPRAIFY

SEQ ID NO: 457 - protein, 9 aa, Homo sapiens
FSEPRAVFY

SEQ ID NO: 458 - protein, 10 aa, Homo sapiens
  MISC_FEATURE (2)..(2): Xaa is Ile or Thr
VXDSAELQAY

SEQ ID NO: 459 - protein, 10 aa, Homo sapiens
VIDSAELQAY

SEQ ID NO: 460 - protein, 10 aa, Homo sapiens
VTDSAELQAY

SEQ ID NO: 461 - protein, 10 aa, Homo sapiens
  MISC_FEATURE (6)..(6): Xaa is Gln or Lys
LPRGMXPTEF

SEQ ID NO: 462 - protein, 10 aa, Homo sapiens
LPRGMQPTEF

SEQ ID NO: 463 - protein, 10 aa, Homo sapiens
LPRGMKPTEF

SEQ ID NO: 464 - protein, 9 aa, Homo sapiens
  MISC_FEATURE (8)..(8): Xaa is Lys or Arg
NSEEHSAXY

SEQ ID NO: 465 - protein, 9 aa, Homo sapiens
NSEEHSAKY

SEQ ID NO: 466 - protein, 9 aa, Homo sapiens
NSEEHSARY

SEQ ID NO: 467 - protein, 9 aa, Homo sapiens
  MISC_FEATURE (5)..(5): Xaa is Arg or Trp
TTDKXTSFY

SEQ ID NO: 468 - protein, 9 aa, Homo sapiens
TTDKRTSFY

SEQ ID NO: 469 - protein, 9 aa, Homo sapiens
TTDKWTSFY

SEQ ID NO: 470 - protein, 10 aa, Homo sapiens
  MISC_FEATURE (1)..(1): Xaa is Ser or Gly
XEMDRRNDAW

SEQ ID NO: 471 - protein, 10 aa, Homo sapiens
SEMDRRNDAW

SEQ ID NO: 472 - protein, 10 aa, Homo sapiens
GEMDRRNDAW

SEQ ID NO: 473 - protein, 9 aa, Homo sapiens
  MISC_FEATURE (1)..(1): Xaa is Arg or Cys
XPTRKPLSL

SEQ ID NO: 474 - protein, 9 aa, Homo sapiens
RPTRKPLSL

SEQ ID NO: 475 - protein, 9 aa, Homo sapiens
CPTRKPLSL

SEQ ID NO: 476 - protein, 10 aa, Homo sapiens
  MISC_FEATURE (7)..(7): Xaa is Ile or Val
YTDSSSXLNY

SEQ ID NO: 477 - protein, 10 aa, Homo sapiens
YTDSSSILNY

SEQ ID NO: 478 - protein, 10 aa, Homo sapiens
YTDSSSVLNY

SEQ ID NO: 479 - protein, 11 aa, Homo sapiens
  MISC_FEATURE (4)..(4): Xaa is Lys or Asn
SPGXERHLNAL

SEQ ID NO: 480 - protein, 11 aa, Homo sapiens
SPGKERHLNAL

SEQ ID NO: 481 - protein, 11 aa, Homo sapiens
SPGNERHLNAL

SEQ ID NO: 482 - protein, 14 aa, Homo sapiens
  MISC_FEATURE (2)..(2): Xaa is Thr, Arg or Ile
FXESRVSSQQTVSY

SEQ ID NO: 483 - protein, 14 aa, Homo sapiens
FTESRVSSQQTVSY

SEQ ID NO: 484 - protein, 14 aa, Homo sapiens
FRESRVSSQQTVSY

SEQ ID NO: 485 - protein, 14 aa, Homo sapiens
FIESRVSSQQTVSY

SEQ ID NO: 486 - protein, 9 aa, Homo sapiens
  MISC_FEATURE (9)..(9): Xaa is Val or Ile
KETDVVLKX

SEQ ID NO: 487 - protein, 9 aa, Homo sapiens
KETDVVLKV

SEQ ID NO: 488 - protein, 9 aa, Homo sapiens
KETDVVLKI

SEQ ID NO: 489 - protein, 9 aa, Homo sapiens
  MISC_FEATURE (7)..(7): Xaa is Ile or Met
REEPEKXIL

SEQ ID NO: 490 - protein, 9 aa, Homo sapiens
REEPEKIIL

SEQ ID NO: 491 - protein, 9 aa, Homo sapiens
REEPEKMIL

SEQ ID NO: 492 - protein, 9 aa, Homo sapiens
  MISC_FEATURE (1)..(1): Xaa is Met, Leu or Val
XELQQKAEF

SEQ ID NO: 493 - protein, 9 aa, Homo sapiens
MELQQKAEF

SEQ ID NO: 494 - protein, 9 aa, Homo sapiens
LELQQKAEF

SEQ ID NO: 495 - protein, 9 aa, Homo sapiens
VELQQKAEF

SEQ ID NO: 496 - protein, 9 aa, Homo sapiens
  MISC_FEATURE (6)..(6): Xaa is Arg or Lys
QEEQTXVAL

SEQ ID NO: 497 - protein, 9 aa, Homo sapiens
QEEQTRVAL

SEQ ID NO: 498 - protein, 9 aa, Homo sapiens
QEEQTKVAL

SEQ ID NO: 499 - protein, 9 aa, Homo sapiens
  MISC_FEATURE (7)..(7): Xaa is Val or Ile
ATFYGPXKK

SEQ ID NO: 500 - protein, 9 aa, Homo sapiens
ATFYGPVKK

SEQ ID NO: 501 - protein, 9 aa, Homo sapiens
ATFYGPIKK

SEQ ID NO: 502 - protein, 10 aa, Homo sapiens
  MISC_FEATURE (1)..(1): Xaa is Glu or Gln
XETAIYKGDY

SEQ ID NO: 503 - protein, 10 aa, Homo sapiens
EETAIYKGDY

SEQ ID NO: 504 - protein, 10 aa, Homo sapiens
QETAIYKGDY

SEQ ID NO: 505 - protein, 10 aa, Homo sapiens
  MISC_FEATURE (7)..(7): Xaa is Met or Thr
ATSNVHXVKK

SEQ ID NO: 506 - protein, 10 aa, Homo sapiens
ATSNVHMVKK

SEQ ID NO: 507 - protein, 10 aa, Homo sapiens
ATSNVHTVKK

SEQ ID NO: 508 - protein, 9 aa, Homo sapiens
  MISC_FEATURE (7)..(7): Xaa is Arg or Ile
EEINLQXNI

SEQ ID NO: 509 - protein, 9 aa, Homo sapiens
EEINLQRNI

SEQ ID NO: 510 - protein, 9 aa, Homo sapiens
EEINLQINI

SEQ ID NO: 511 - protein, 9 aa, Homo sapiens
  MISC_FEATURE (2)..(2): Xaa is Glu or Asp
QXLIGKKEY

SEQ ID NO: 512 - protein, 9 aa, Homo sapiens
QELIGKKEY

SEQ ID NO: 513 - protein, 9 aa, Homo sapiens
QDLIGKKEY

SEQ ID NO: 514 - protein, 9 aa, Homo sapiens
  MISC_FEATURE (2)..(2): Xaa is Pro, Leu or Arg
RXAGPALLL

SEQ ID NO: 515 - protein, 9 aa, Homo sapiens
RPAGPALLL

SEQ ID NO: 516 - protein, 9 aa, Homo sapiens
RLAGPALLL

SEQ ID NO: 517 - protein, 9 aa, Homo sapiens
RRAGPALLL

SEQ ID NO: 518 - protein, 9 aa, Homo sapiens
  MISC_FEATURE (3)..(3): Xaa is Ala, Thr or Ser
EEXPSQQGF

SEQ ID NO: 519 - protein, 9 aa, Homo sapiens
EEAPSQQGF

SEQ ID NO: 520 - protein, 9 aa, Homo sapiens
EETPSQQGF

SEQ ID NO: 521 - protein, 9 aa, Homo sapiens
EESPSQQGF

(19) United States
(12) Patent Application Publication
(10) Pub. No.: US 2020/0055918 A1
(43) Pub. Date: Feb. 20, 2020

(54) NOVEL MINOR HISTOCOMPATIBILITY ANTIGENS AND USES THEREOF

(71) Applicant: UNIVERSITÉ DE MONTRÉAL, Montréal (CA)

(72) Inventors: Claude Perreault, Outremont (CA); Pierre Thibault, Montréal (CA); Sébastien Lemieux, Lasalle (CA); Krystel Vincent, Terrebonne (CA)

(21) Appl. No.: 16/487,604

(22) PCT Filed: Feb. 22, 2018
(86) PCT No.: PCT/CA2018/050201

§ 371 Date: Aug. 21, 2019

Related U.S. Application Data
(60) Provisional application No. 62/462,035, filed on Feb. 22, 2017.

(51) Int. Cl. C07K 14/74; A61P 35/00; A61P 37/04; C07K 7/06; C07K 7/08; A61K 39/00

(52) U.S. Cl. CPC C07K 14/70539; A61P 35/00; A61P 37/04; A61K 2039/605; C07K 7/08; A61K 39/001114; C07K 7/06

(57) ABSTRACT

Minor histocompatibility antigens (MiHAs) binding to certain human leukocyte antigen (HLA) alleles are described. These MiHAs were selected based on two features: (i) they are encoded by loci with a minor allele frequency (MAF) of at least 0.05; and (ii) they have adequate tissue distribution. Compositions, nucleic acids and cells related to these MiHAs are also described. The present application also discloses the use of these MiHAs, and related compositions, nucleic acids and cells, in applications related to cancer immunotherapy, for example for the treatment of hematologic cancers such as leukemia.

### CROSS-REFERENCE TO RELATED APPLICATIONS

The present application claims the benefit of U.S. provisional application Ser. No. 62/462,035 filed on Feb. 22, 2017, which is incorporated herein by reference in its entirety.

### TECHNICAL FIELD

The present disclosure generally relates to histocompatibility antigens, and more specifically to minor histocompatibility antigens (MiHAs) and use thereof, for example in immunotherapies.

### BACKGROUND ART

While several treatment modalities have proven effective for cancer immunotherapy, cancer immunotherapists will undoubtedly need more than one weapon in their therapeutic armamentarium. In particular, different approaches are required for tumors with high vs. low mutation loads.¹ Solid tumors induced by carcinogens (e.g., melanoma, lung cancer) express numerous mutations that create tumor-specific antigens (TSAs) which can be targeted using two approaches: injection of ex vivo expanded tumor-infiltrating lymphocytes and administration of antibodies against checkpoint molecules.¹⁻³ However, TSAs are exceedingly rare on hematologic cancers (HCs), because of their very low mutation load, and alternative targets must therefore be found for immunotherapy of HCs.¹ T cells redirected to CD19 or CD20 antigen targets with engineered chimeric antigen receptors are spectacularly effective for treatment of B-cell malignancies and represent a breakthrough in cancer immunotherapy.^{4,5} However, whether chimeric antigen receptors might be used for treatment of myeloid malignancies remains a matter of speculation.⁶

Major histocompatibility complex (MHC) molecules are transmembrane glycoproteins encoded by closely linked polymorphic loci located on chromosome 6 in humans. Their primary role is to bind peptides and present them to T cells. MHC molecules (human leukocyte antigen or HLA in humans) present thousands of peptides at the surface of human cells. These MHC-associated peptides (MAPs) are referred to as the immunopeptidome. The normal immunopeptidome derived from self-proteins of identical twins (AKA syngeneic individuals) is identical. By contrast, MAPs derived from self-proteins present on cells from HLA-identical non-syngeneic individuals are classified into two categories: i) monomorphic MAPs which originate from invariant genomic regions and are therefore present in all individuals with a given HLA type, and ii) polymorphic MAPs (AKA MiHAs) which are encoded by polymorphic genomic regions and are therefore present in some individuals but absent in other individuals. MiHAs are essentially genetic polymorphisms viewed from a T-cell perspective. MiHAs are typically encoded by bi-allelic loci and where each allele can be dominant (generates a MAP) or recessive (generates no MAP). Indeed, a non-synonymous single nucleotide polymorphism (ns-SNP) in a MAP-coding genomic sequence will either hinder MAP generation (recessive allele) or generate a variant MAP (dominant allele). Another strategy that can be used for cancer immunotherapy is adoptive T-cell immunotherapy (ATCI). The term “ATCI” refers to transfusing a patient with T lymphocytes obtained from: the patient (autologous transfusion), a genetically-identical twin donor (syngeneic transfusion), or a non-identical HLA-compatible donor (allogeneic transfusion). To date, ATCI has yielded much higher cancer remission and cure rates than vaccines, and the most widely used form of cancer ATCI is allogeneic hematopoietic cell transplantation (AHCT). The so-called graft-versus-leukemia (GVL) effect induced by allogeneic hematopoietic cell transplantation (AHCT) is due mainly to T-cell responses against host MiHAs: the GVL is abrogated or significantly reduced if the donor is an identical twin (no MiHA differences with the recipient) or if the graft is depleted of T lymphocytes. More than 400,000 individuals treated for hematological cancers owe their life to the MiHA-dependent GVL effect which represents the most striking evidence of the ability of the human immune system to eradicate neoplasia. Though the allogeneic GVT effect is being used essentially to treat patients with hematologic malignancies, preliminary evidence suggests that it may be also effective for the treatment of solid tumors. The considerable potential of MiHA-targeted cancer immunotherapy has not been properly exploited in medicine. In current medical practice, MiHA-based immunotherapy is limited to “conventional” AHCT, that is, injection of hematopoietic cells from an allogeneic HLA-matched donor. Such unselective injection of allogeneic lymphocytes is a very rudimentary form of MiHA-targeted therapy. First, it lacks specificity and is therefore highly toxic: unselected allogeneic T cells react against a multitude of host MiHAs and thereby induce graft-versus-host-disease (GVHD) in 60% of recipients. GVHD is always incapacitating and frequently lethal. Second, conventional AHCT induces only an attenuated form of GVT reaction because donor T cells are not being primed (pre-activated) against specific MiHAs expressed on cancer cells prior to injection into the patient. While primed T cells are resistant to tolerance induction, naïve T cells can be tolerized by tumor cells. It has been demonstrated in mice models of AHCT that, by replacing unselected donor lymphocytes with CD8⁺ T cells primed against a single MiHA, it was possible to cure leukemia and melanoma without causing GVHD or any other untoward effect. Success depends on two key elements: selection of an immunogenic MiHA expressed on neoplastic cells, and priming of donor CD8⁺ T cells against the target MiHA prior to AHCT. A recent report discusses why MiHA-targeted ATCI is so effective and how translation of this approach in the clinic could have a tremendous impact on cancer immunotherapy⁸. High-avidity T cell responses capable of eradicating tumors can be generated in an allogeneic setting. In hematological malignancies, allogeneic HLA-matched hematopoietic stem cell transplantation (ASCT) provides a platform for allogeneic immunotherapy due to the induction of T cell-mediated graft-versus-tumor (GVT) immune responses. Immunotherapy in an allogeneic setting enables induction of effective T cell responses due to the fact that T cells of donor origin are not selected for low reactivity against self-antigens of the recipient. Therefore, high-affinity T cells against tumor- or recipient-specific antigens can be found in the T cell inoculum administered to the patient during or after ASCT. The main targets of the tumor-reactive T cell responses are polymorphic proteins for which donor and recipient are disparate, namely MiHAs. However, implementation of MiHA-targeted immunotherapy in humans has been limited mainly by the paucity of molecularly defined human MiHAs. Based on the MiHAs currently known, only 33% of patients with leukemia would be eligible for MiHA-based ATCI. MiHA discovery is a difficult task because it cannot be achieved using standard genomic and proteomic methods. Indeed, i) less than 1% of SNPs generate a MiHA and ii) current mass spectrometry methods cannot detect MiHAs. Thus, there is a need for the identification of MiHAs that may be used in immunotherapies.

The present description refers to a number of documents, the content of which is herein incorporated by reference in their entirety.

### SUMMARY OF THE INVENTION

The present disclosure relates to the following items 1 to 65:

1. A Minor Histocompatibility Antigen (MiHA) peptide of 8 to 14 amino acids of the formula I

Z¹—X¹—Z² (I)

wherein
Z¹ is an amino terminal modifying group or is absent;
X¹ is a sequence comprising at least 8 contiguous residues of one of the peptide sequences of MiHAs Nos. 3, 2, 1 and 4-138 or MiHAs Nos. 3, 2, 1 and 4-81, preferably MiHAs Nos. 3, 5, 8-15, 25-28, 30-33, 36-49, 54-61, 65-66, 68-77 and 79-81 set forth in Table I and comprising the polymorphic amino acid depicted; and
Z² is a carboxy terminal modifying group or is absent.
2. The MiHA peptide of item 1, wherein X¹ consists of any one of the peptide sequences of MiHAs Nos. 3, 5, 8-15, 25-28, 30-33, 36-49, 54-61, 65-66, 68-77 and 79-81 set forth in Table I.
3. The MiHA peptide of item 1 or 2, wherein Z¹ is absent.
4. The MiHA peptide of any one of items 1 to 3, wherein Z² is absent.
5. The MiHA peptide of any one of items 1 to 4, wherein said MiHA peptide consists of any one of the peptide sequences of MiHAs Nos. 3, 5, 8-15, 25-28, 30-33, 36-49, 54-61, 65-66, 68-77 and 79-81 set forth in Table I.
6. The MiHA peptide of any one of items 1 to 5, wherein said MiHA derives from a locus with a minor allele frequency (MAF) of at least 0.1.
7. The MiHA peptide of item 6, wherein said MiHA derives from a locus with a minor allele frequency (MAF) of at least 0.2.
8. The MiHA peptide of any one of items 1 to 7, wherein said MiHA peptide binds to a major histocompatibility complex (MHC) class I molecule of the HLA-A*01:01 allele, wherein X¹ is a sequence of at least 8 amino acids of any one of the MiHA Nos. 5, 47 and 81 set forth in Table I, wherein said sequence comprises the polymorphic amino acid depicted.
9. The MiHA peptide of any one of items 1 to 7, wherein said peptide binds to a major histocompatibility complex (MHC) class I molecule of the HLA-A*03:01 allele, wherein X¹ is a sequence of at least 8 amino acids of any one of the MiHA Nos. 36 and 77 set forth in Table I, wherein said sequence comprises the polymorphic amino acid depicted.
10. The MiHA peptide of any one of items 1 to 7, wherein said peptide binds to a major histocompatibility complex (MHC) class I molecule of the HLA-A*11:01 allele, wherein X¹ is a sequence of at least 8 amino acids of any one of the MiHA Nos. 1, 3, 13, 31, 61, 62 and 69 set forth in Table I, wherein said sequence comprises the polymorphic amino acid depicted.
11. The MiHA peptide of any one of items 1 to 7, wherein said peptide binds to a major histocompatibility complex (MHC) class I molecule of the HLA-A*24:02 allele, wherein X¹ is a sequence of at least 8 amino acids of any one of the MiHA Nos. 33, 39, 40 and 79 set forth in Table I, wherein said sequence comprises the polymorphic amino acid depicted.
12. The MiHA peptide of any one of items 1 to 7, wherein said peptide binds to a major histocompatibility complex (MHC) class I molecule of the HLA-A*29:02 allele, wherein X¹ is a sequence of at least 8 amino acids of MiHA No. 21 set forth in Table I, wherein said sequence comprises the polymorphic amino acid depicted.
13. The MiHA peptide of any one of items 1 to 7, wherein said peptide binds to a major histocompatibility complex (MHC) class I molecule of the HLA-A*32:01 allele, wherein X¹ is a sequence of at least 8 amino acids of MiHA No. 55 set forth in Table I, wherein said sequence comprises the polymorphic amino acid depicted.
14. The MiHA peptide of any one of items 1 to 7, wherein said peptide binds to a major histocompatibility complex (MHC) class I molecule of the HLA-B*07:02 allele, wherein X¹ is a sequence of at least 8 amino acids of any one of the MiHA Nos. 8-12, 26, 28, 42, 43, 45, 46, 48, 49, 56-59, 65, 66, 70, 73, 74 set forth in Table I, wherein said sequence comprises the polymorphic amino acid depicted.
15. The MiHA peptide of any one of items 1 to 7, wherein said peptide binds to a major histocompatibility complex (MHC) class I molecule of the HLA-B*08:01 allele, wherein X¹ is a sequence of at least 8 amino acids of any one of the MiHA Nos. 25, 27 and 71 set forth in Table I, wherein said sequence comprises the polymorphic amino acid depicted.
16. The MiHA peptide of any one of items 1 to 7, wherein said peptide binds to a major histocompatibility complex (MHC) class I molecule of the HLA-B*13:02 allele, wherein X¹ is a sequence of at least 8 amino acids of MiHA No. 67 set forth in Table I, wherein said sequence comprises the polymorphic amino acid depicted.
17. The MiHA peptide of any one of items 1 to 7, wherein said peptide binds to a major histocompatibility complex (MHC) class I molecule of the HLA-B*14:02 allele, wherein X¹ is a sequence of at least 8 amino acids of any one of the MiHA Nos. 14, 15 and 44 set forth in Table I, wherein said sequence comprises the polymorphic amino acid depicted.
18. The MiHA peptide of any one of items 1 to 7, wherein said peptide binds to a major histocompatibility complex (MHC) class I molecule of the HLA-B*15:01 allele, wherein X¹ is a sequence of at least 8 amino acids of any one of the MiHA Nos. 38, 40, 72 and 76 set forth in Table I, wherein said sequence comprises the polymorphic amino acid depicted.
19. The MiHA peptide of any one of items 1 to 7, wherein said peptide binds to a major histocompatibility complex (MHC) class I molecule of the HLA-B*18:01 allele, wherein X¹ is a sequence of at least 8 amino acids of any one of the MiHA Nos. 2, 20, 34, 41, 50, 52 and 54 set forth in Table I, wherein said sequence comprises the polymorphic amino acid depicted.
20. The MiHA peptide of any one of items 1 to 7, wherein said peptide binds to a major histocompatibility complex (MHC) class I molecule of the HLA-B*27:05 allele, wherein X¹ is a sequence of at least 8 amino acids of any one of the MiHA Nos. 1, 30, 32, 37, 65 and 68 set forth in Table I, wherein said sequence comprises the polymorphic amino acid depicted.
21. The MiHA peptide of any one of items 1 to 7, wherein said peptide binds to a major histocompatibility complex (MHC) class I molecule of the HLA-B*35:01 allele, wherein X¹ is a sequence of at least 8 amino acids of MiHA No. 75 set forth in Table I, wherein said sequence comprises the polymorphic amino acid depicted.
22. The MiHA peptide of any one of items 1 to 7, wherein said peptide binds to a major histocompatibility complex (MHC) class I molecule of the HLA-B*40:01 allele, wherein X¹ is a sequence of at least 8 amino acids of any one of the MiHA Nos. 2, 19, 21, 22, 29, 34, 35, 52 and 64 set forth in Table I, wherein said sequence comprises the polymorphic amino acid depicted.
23. The MiHA peptide of any one of items 1 to 7, wherein said peptide binds to a major histocompatibility complex (MHC) class I molecule of the HLA-B*44:02 allele, wherein X¹ is a sequence of at least 8 amino acids of any one of the MiHA Nos. 2, 4, 6, 7, 16-24, 29, 34, 35, 50-53, 63, 64 and 78 set forth in Table I, wherein said sequence comprises the polymorphic amino acid depicted.
24. The MiHA peptide of any one of items 1 to 7, wherein said peptide binds to a major histocompatibility complex (MHC) class I molecule of the HLA-B*57:01 allele, wherein X¹ is a sequence of at least 8 amino acids of MiHA No. 34 set forth in Table I, wherein said sequence comprises the polymorphic amino acid depicted.
25. A polypeptide comprising an amino acid sequence of at least one of the MiHA peptide defined in any one of items 1 to 24, wherein said polypeptide is of the following formula Ia:

Z¹—X²—X¹—X³—Z² (Ia)

wherein
Z¹, X¹ and Z² are as defined in any one of items 1 to 24; and
X² and X³ are each independently absent or a sequence of one or more amino acids, wherein said polypeptide does not comprise or consist of an amino acid sequence of a native protein, and wherein processing of said polypeptide by a cell results in the loading of the MiHA peptide in the peptide-binding groove of MHC class I molecules expressed by said cell.
26. A peptide combination comprising (i) at least two of the MiHA peptides defined in any one of items 1 to 24; or (ii) at least one of the MiHA peptides defined in any one of items 1 to 24 and at least one additional MiHA peptide.
27. A nucleic acid encoding the MiHA peptide of any one of items 1 to 24, or the polypeptide of item 25.
28. The nucleic acid of item 27, which is present in a plasmid or a vector.
29. An isolated major histocompatibility complex (MHC) class I molecule comprising the MiHA peptide of any one of items 1 to 24 in its peptide binding groove.
30. The isolated MHC class I molecule of item 29, which is in the form of a multimer.
31. The isolated MHC class I molecule of item 30, wherein said multimer is a tetramer.
32. An isolated cell comprising the MiHA peptide of any one of items 1 to 24, the polypeptide of item 25, the peptide combination of item 26, or the nucleic acid of item 27 or 28.
33. An isolated cell expressing at its surface major histocompatibility complex (MHC) class I molecules comprising the MiHA peptide of any one of items 1 to 24, or the peptide combination of item 26, in their peptide binding groove.
34. The cell of item 33, which is an antigen-presenting cell (APC).
35. The cell of item 34, wherein said APC is a dendritic cell.
36. A T-cell receptor (TCR) that specifically recognizes the isolated MHC class I molecule of any one of items 29-31 and/or MHC class I molecules expressed at the surface of the cell of any one of items 32-35.
37. One or more nucleic acids encoding the alpha and beta chains of the TCR of item 36.
38. The one or more nucleic acids of item 37, which are present in a plasmid or a vector.
39. An isolated CD8⁺ T lymphocyte expressing at its cell surface the TCR of item 36.
40. The CD8⁺ T lymphocyte of item 39, which is transfected or transduced with the one or more nucleic acids of item 37 or 38.
41. A cell population comprising at least 0.5% of CD8⁺ T lymphocytes as defined in item 39 or 40.
42. A composition comprising (i) the MiHA peptide of any one of items 1 to 24; (ii) the polypeptide of item 25; (iii) the peptide combination of item 26; (iv) the nucleic acid of item 27 or 28; (iv) the MHC class I molecule of any one of items 29-31; (v) the cell of any one of 32-35; (v) the TCR of item 36; (vi) the one or more nucleic acids of item 37 or 38; the CD8⁺ T lymphocyte of item 39 or 40; and/or (vii) the cell population of item 41.
43. The composition of item 42, further comprising a buffer, an excipient, a carrier, a diluent and/or a medium.
44. The composition of item 42 or 43, wherein said composition is a vaccine and further comprises an adjuvant.
45. The composition of any one of items 42 to 44, wherein said composition comprises the peptide combination of item 26, or one or more nucleic acids encoding the at least two MiHA peptides present in said peptide combination.
46. The composition of any one of items 42 to 45, which comprises the cell of any one of items 32-35 and the CD8⁺ T lymphocyte of item 38 or 39.
47. A method of expanding CD8⁺ T lymphocytes specifically recognizing one or more of the MiHA peptides defined in any one of items 1 to 24, said method comprising culturing, under conditions suitable for CD8⁺ T lymphocyte expansion, CD8⁺ T lymphocytes from a candidate donor that does not express said one or more MiHA peptides in the presence of cells according to any one of items 32-35.
48. A method of treating cancer, said method comprising administering to a subject in need thereof an effective amount of (i) the CD8⁺ T lymphocytes of item 39 or 40; (ii) the cell population of item 41; and/or (iii) a composition comprising (i) or (ii).
49. The method of item 48, said method further comprising determining one or more MiHA variants expressed by said subject in need thereof, wherein the CD8⁺ T lymphocytes specifically recognize said one or more MiHA variants presented by MHC class I molecules.
50. The method of item 49, wherein said determining comprises sequencing a nucleic acid encoding said MiHA.
51. The method of any one of items 48 to 50, wherein said CD8⁺ T lymphocytes are ex vivo expanded CD8⁺ T lymphocytes prepared according to the method of item 47.
52. The method of any one of items 48 to 51, wherein said method further comprises expanding CD8⁺ T lymphocytes according to the method of item 47.
53. The method of any one of items 48 to 52, wherein said subject in need thereof is an allogeneic stem cell transplantation (ASCT) recipient.
54. The method of any one of items 48 to 53, further comprising administering an effective amount of the MiHA peptide recognized by said CD8⁺ T lymphocytes, and/or (ii) a cell expressing at its surface MHC class I molecules comprising the MiHA peptide defined in (i) in their peptide binding groove.
55. The method of any one of items 48 to 54, wherein said cancer is a hematologic cancer.
56. The method of item 55, wherein said hematologic cancer is a leukemia, a lymphoma or a myeloma.
57. An antigen presenting cell or an artificial construct mimicking an antigen-presenting cell that presents the MiHA peptide of any one of items 1 to 24 or the peptide combination of item 26.
58. An in vitro method for producing cytotoxic T lymphocytes (CTLs) comprising contacting a T lymphocyte with human class I MHC molecules loaded with the MiHA peptide of any one of items 1 to 24 or the peptide combination of item 26 expressed on the surface of a suitable antigen presenting cell or an artificial construct mimicking an antigen-presenting cell for a period of time sufficient to activate said T lymphocyte in an antigen-specific manner.
59. An activated cytotoxic T lymphocyte obtained by method of item 58.
60. A method of treating a subject with haematological cancer comprising administering to the patient an effective amount of the cytotoxic T lymphocyte of item 59.
61. A method of generating immune response against tumor cells expressing human class I MHC molecules loaded with the MiHA peptide of any one of items 1 to 24 or the peptide combination of item 26 in a subject, said method comprising administering the cytotoxic T lymphocyte of item 59.
62. An antigen presenting cell (APC) artificially loaded with one or more of the MiHA peptides defined in any one of items 1 to 24, or the peptide combination of item 26.
63. The APC of item 62 for use as a therapeutic vaccine.
64. A method for generating an immune response in a subject comprising administering to the subject allogenic T lymphocytes and a composition comprising one or more of the MiHA peptides defined in any one of items 1 to 24, or the peptide combination of item 26.
65. The method of any one of items 60, 61 and 64, wherein said subject has a haematological cancer selected from leukemia, lymphoma and myeloma.

Other objects, advantages and features of the present invention will become more apparent upon reading of the following non-restrictive description of specific embodiments thereof, given by way of example only.

### BRIEF DESCRIPTION OF DRAWINGS

In the appended drawings:

FIGS. 1A to 1D show the MiHA peptides described in PCT publication Nos. WO/2016/127249 (FIG. 1A) and WO/2014/026277 (FIG. 1B), Spaapen and Mutis, Best Practice & Research Clinical Hematology, 21(3): 543-557 (FIG. 1C), and Akatsuka et al., Cancer Sci, 98(8): 1139-1146, 2007 (FIG. 1D). FIG. 1C is derived from Table 1 of Spaapen and Mutis, and FIG. 1D is derived from Table 1 of Akatsuka et al.

### DISCLOSURE OF INVENTION

Terms and symbols of genetics, molecular biology, biochemistry and nucleic acid used herein follow those of standard treatises and texts in the field, e.g. Kornberg and Baker, DNA Replication, Second Edition (W University Science Books, 2005); Lehninger, Biochemistry, sixth Edition (W H Freeman & Co (Sd), New York, 2012); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

The articles “a” and “an” are used herein to refer to one or to more than one (i.e. to at least one) of the grammatical object of the article. By way of example, “an element” means one element or more than one element. Throughout this specification, unless the context requires otherwise, the words “comprise,” “comprises” and “comprising” will be understood to imply the inclusion of a stated step or element or group of steps or elements but not the exclusion of any other step or element or group of steps or elements. The terms “subject”, “patient” and “recipient” are used interchangeably herein, and refer to an animal, preferably a mammal, most preferably a human, who is in the need of treatment for cancer using one or more MiHAs as described herein. The term “individual” refers to an animal, preferably a mammal, most preferably a human, who does not have cancer (i.e. healthy). These terms encompass both adults and children. A “donor” is either a cancer patient (in case of autogenic cell transfusion), or a healthy patient (in case of allogenic cell transfusion).

### MiHA Peptides and Nucleic Acids

In an aspect, the present disclosure provides a polypeptide (e.g., an isolated or synthetic polypeptide) comprising an amino acid sequence of a MiHA peptide, wherein said polypeptide is of the following formula Ia:

Z¹—X²—X¹—X³—Z² (Ia)

wherein
Z¹, X¹ and Z² are as defined below; and
X² and X³ are each independently absent or a sequence of one or more amino acids, wherein said polypeptide does not comprise or consist of an amino acid sequence of a native protein (e.g., the amino acid sequence of the native protein from which the MiHA peptide is derived), and wherein processing of said polypeptide by a cell (e.g., an antigen-presenting cell) results in the loading of the MiHA peptide of sequence X¹ in the peptide-binding groove of MHC class I molecules expressed by said cell.

In an embodiment, X² and/or X³ are each independently a sequence of about 1 to about 5, 10, 15, 20, 25, 30, 40, 50, 100, 200, 300, 400, 500 or 1000 amino acids. In an embodiment, X² is a sequence of amino acids that is immediately amino-terminal to the sequence of X¹ in the native polypeptide from which the MiHA is derived (see Table II for the Ensembl gene ID corresponding to the gene from which the MiHA described herein are derived). In an embodiment, X³ is a sequence of amino acids that is immediately carboxy-terminal to the sequence of X¹ in the native polypeptide from which the MiHA is derived (see Table II). For example, MiHA No. 2 derives from the protein Ras association domain family member 1 (RASSF1), and thus X² and/or X³ may comprises the one or more amino acids immediately amino- and/or carboxy-terminal to the sequence A/SEIEQKIKEY in RASSF1 (Ensembl gene ID No. ENSG00000068028, NCBI Reference Sequence: NP_009113). Thus, the sequences immediately amino- and/or carboxy-terminal to the sequences of the MiHAs described herein may be easily identified using the information available in public databases such as Ensembl, NCBI, UniProt, which may be retrieved for example using the SNP ID Nos. and/or Ensembl gene ID Nos. provided in Table II below. The entire content and information, including the full sequences of all the transcripts and encoded polypeptides, corresponding to the SNP ID Nos. and Ensembl gene ID Nos. provided herein (e.g., in Table II), are incorporated herein by reference.

In another embodiment, X² and/or X³ are absent. In a further embodiment, X² and X³ are both absent.

Thus, in another aspect, the present disclosure provides a MiHA peptide (e.g., an isolated or synthetic peptide) of about 8 to about 14 amino acids of formula I

Z¹—X¹—Z² (I)

wherein Z¹ is an amino terminal modifying group or is absent; X¹ is a sequence comprising at least 8 (preferably contiguous) residues of one of the peptide sequences of MiHA Nos. 1-81, preferably MiHA Nos. 3, 5, 8-15, 25-28, 30-33, 36-49, 54-61, 65-66, 68-77 and 79-81, set forth in Table I below and comprising the polymorphic amino acid (variation) depicted (underlined, e.g., for MiHA No. 2, the N-terminal residue A or S is comprised in X¹ and for MiHA No. 3, the residue P or H is comprised in domain X¹, etc.); and Z² is a carboxy terminal modifying group or is absent. The reference to MiHA Nos. 1-81 encompasses each of the variants defined by the sequences depicted. For example, the term “MiHA No. 2” (A/SEIEQKIKEY, SEQ ID NO: 4) refers to AEIEQKIKEY (SEQ ID NO: 5) and/or SEIEQKIKEY (SEQ ID NO: 6).

TABLE I
Sequences of MiHAs described herein

| MiHA | | SEQ ID |
|---|---|---|
| No. | Sequence | No. |
| 1 | R/*VWDLPGVLK | 1-3 |
| 2 | A/SEIEQKIKEY | 4-6 |
| 3 | AAQTARQP/HPK | 7-9 |
| 4 | NESNTQKTY or | 10 |
| | absent^{a} | |
| 5 | QTDPRAGGGGGGDY | 11 |
| | or absent^{b} | |
| 6 | AE/AIQEKKEI | 12-14 |
| 7 | AELQS/APLAA | 15-17 |
| 8 | APPAEKA/VPV | 18-20 |
| 9 | APREP/QFAHSL | 21-23 |
| 10 | APRES/NAQAI | 24-26 |
| 11 | APRPFGSVF/S | 27-29 |
| 12 | APRR/CPPPPP | 30-32 |
| 13 | AQTARQP/HPK | 33-35 |
| 14 | DRANRFEY/*L | 36-38 |
| 15 | DRFVARK/R/M/TL | 39-43 |
| 16 | EE/GRGENTSY | 44-46 |
| 17 | EEADGN/HKQWW | 47-49 |
| 18 | EEALGLYH/QW | 50-52 |
| 19 | EEINLQR/INI | 53-55 |
| 20 | EEIPV/ISSHY | 56-58 |
| 21 | EEIPV/ISSHYF | 59-61 |
| 22 | EELLAVG/SKF | 62-64 |
| 23 | EESAVPE/KPSW | 65-67 |
| 24 | EE/KEQSQSPW | 68-70 |
| 25 | ELQA/SRLAAL | 71-73 |
| 26 | EPQGS/FGRQGNSL | 74-76 |
| 27 | ESKIR/CVLL | 77-79 |
| 28 | G/DPRPSPTRSV | 80-82 |
| 29 | GED/GKGIKAL | 83-85 |
| 30 | GRA/EGIVARL | 86-88 |
| 31 | GTLSPSLGNSSI/VLK | 89-91 |
| 32 | HRVYLVRKL/I | 92-94 |
| 33 | IYPQV/LLHSL | 95-97 |
| 34 | KEFEDD/GIINW | 98-100 |
| 35 | KEINEKSN/SIL | 101-103 |
| 36 | KLYSEA/GKTK | 104-106 |
| 37 | KRVGASYER/W/G | 107-110 |
| 38 | KVKTSLNEQM/TY | 111-113 |
| 39 | KY/HMTAVVKL | 114-116 |
| 40 | KY/HMTAVVKLF | 117-119 |
| 41 | LENGAH/RAY | 120-122 |
| 42 | LPRVC/RGTTL | 123-125 |
| 43 | LPSKRVSL/I | 126-128 |
| 44 | LRIQ/HQREQL | 129-131 |
| 45 | MPSHLRNT/ILL | 132-134 |
| 46 | MPSHLRNT/ILLM | 135-137 |
| 47 | NSEEHSAR/KY | 138-140 |
| 48 | PH/PRYRPGTVAL | 141-143 |
| 49 | PPSGLRLLP/LL | 144-146 |
| 50 | QE/DLIGKKEY | 147-149 |
| 51 | QEN/DIQ/HNLQL | 150-154 |
| 52 | QELDG/RVFQKL | 155-157 |
| 53 | QENQDPR/GPW | 158-160 |
| 54 | QERSFQEY/N | 161-163 |
| 55 | R/GIFASRLYY | 164-166 |
| 56 | RANLRAT/A/NKL | 167-170 |
| 57 | RPPG/EGSGPL | 171-173 |
| 58 | RPPG/EGSGPLL/H/R/P | 174-182 |
| 59 | RPPPP/SPAWL | 183-185 |
| 60 | RREDV/IVLGR | 186-188 |
| 61 | RTA/TDNFDDILK | 189-191 |
| 62 | S/TVLKPGNSK | 192-194 |
| 63 | SEESAVPE/KPSW | 195-197 |
| 64 | SESKIR/CVLL | 198-200 |
| 65 | SPD/ESSTPKL | 201-203 |
| 66 | SPRGN/KLPLLL | 204-206 |
| 67 | SQA/SEIEQKI | 207-209 |
| 68 | SRVLQN/KVAF | 210-212 |
| 69 | SVSKLST/NPK | 213-215 |
| 70 | T/PARPQSSAL | 216-218 |
| 71 | TAKQKLDPA/V | 219-221 |
| 72 | TLN/SERFTSY | 222-224 |
| 73 | TPRNTYKMTSL/V | 225-227 |
| 74 | TPRPIQSSL/P | 228-230 |
| 75 | TPVDDR/SSL | 231-233 |
| 76 | TQR/SPADVIF | 234-236 |
| 77 | TVY/CHSPVSR | 237-239 |
| 78 | VEEADGN/HKQW | 240-242 |
| 79 | VYNNIMRH/RYL | 243-245 |
| 80 | YPRAGS/RKPP | 246-248 |
| 81 | YTDSSSI/VLNY | 249-251 |
| 82 | APKKPTGA/VDL | 348-350 |
| 83 | ASELHTSLH/Y | 351-353 |
| 84 | EEV/LKLRQQL | 354-356 |
| 85 | EL/IDPSNTKALY | 357-359 |
| 86 | EI/LDPSNTKALY | 357-359 |
| 87 | VPNV/EKSGAL | 360-362 |
| 88 | IS/PRAAAERSL | 363-365 |
| 89 | LPSDDRGP/SL | 366-368 |
| 90 | LC/SEKPTVTTVY | 369-371 |
| 91 | RPRAPRES/NAQAI | 373-375 |
| 92 | H/RESPIFKQF | 376-378 |
| 93 | TPRNTYKMTSL/V | 379-381 |
| 94 | VPREYI/VRAL | 382-384 |
| 95 | RPRARYYI/VQV | 385-387 |
| 96 | SAFADRPS/AF | 388-390 |
| 97 | V/APEEARPAL | 391-393 |
| 98 | NLDKNTV/MGY | 394-396 |
| 99 | SPRV/APVSPLKF | 397-399 |
| 100 | SL/PRPQGLSNPSTL | 400-402 |
| 101 | SPRA/VPVSPLKF | 397-399 |
| 102 | TPRPIQSSP/L | 403-405 |
| 103 | HPR/PQEQIAL | 406-408 |
| 104 | YYRTNHT/I/SVM | 409-412 |
| 105 | KEMDSDQQR/T/KSY | 413-416 |
| 106 | M/L/VELQQKAEF | 417-420 |
| 107 | S/YGGPLRSEY | 421-423 |
| 108 | TEAG/AVQKQW | 424-426 |
| 109 | RPR/HPEDQRL | 427-429 |
| 110 | LPRGMQ/KPTEFFQSL | 430-432 |
| 111 | LARPA/VSAAL | 433-435 |
| 112 | APRES/NAQAI | 436-438 |
| 113 | R/QPRAPRESAQAI | 439-441 |
| 114 | RP/LRKEVKEEL | 442-444 |
| 115 | SP/LYPRVKVDF | 445-447 |
| 116 | IPF/LSNPRVL | 448-450 |
| 117 | EEVTS/T/ASEDKRKTY | 451-454 |
| 118 | FSEPRAI/VFY | 455-457 |
| 119 | VI/TDSAELQAY | 458-460 |
| 120 | LPRGMQ/KPTEF | 461-463 |
| 121 | NSEEHSAK/RY | 464-466 |
| 122 | TTDKR/WTSFY | 467-469 |
| 123 | S/GEMDRRNDAW | 470-472 |
| 124 | R/CPTRKPLSL | 473-475 |
| 125 | YTDSSSI/VLNY | 476-478 |
| 126 | SPGK/NERHLNAL | 479-481 |
| 127 | FT/R/IESRVSSQQTVSY | 482-485 |
| 128 | RP/L/RAGPALLL | 514-517 |
| 129 | EEA/T/SPSQQGF | 518-521 |
| 130 | KETDVVLKV/I | 486-488 |
| 131 | REEPEKI/MIL | 489-491 |
| 132 | M/L/VELQQKAEF | 492-495 |
| 133 | QEEQTR/KVAL | 496-498 |
| 134 | ATFYGPV/IKK | 499-501 |
| 135 | E/QETAIYKGDY | 502-504 |
| 136 | ATSNVHM/TVKK | 505-507 |
| 137 | EEINLQR/INI | 508-510 |
| 138 | QE/DLIGKKEY | 511-513 |

Amino acid is absent
^{a}The genes from which this MiHA is derived is located on chromosome Y. Accordingly, this MiHa is present in male but absent in female individuals.
^{b}Deletion mutation (CGC codon) resulting in absence of the MiHA (SNP rs151075597).
MiHA peptides in italics were previously reported but in other HLA alleles.

In another aspect, the present disclosure provides a MiHA peptide of the formula I or Ia as defined herein, wherein X¹ is a sequence comprising at least 8, 9, 10, 11, 12, 13 or 14 amino acids of one of the peptide sequences of MiHAs Nos: 1-138 or MiHAs Nos: 1-81, preferably MiHA Nos. 3, 5, 8-15, 25-28, 30-33, 36-49, 54-61, 65-66, 68-77 and 79-81, and wherein said sequence comprises the polymorphic amino acid depicted.

In an embodiment, the present disclosure provides a peptide pool or combination comprising two, three, four, five, six, seven, eight, nine, ten or more of the MiHA peptides of the formula I or Ia as defined herein.

In another aspect, the present disclosure provides a MiHA peptide of the formula I or Ia as defined herein, wherein X¹ is a sequence comprising at least 8 amino acids of MiHA No. 1 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 2 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 3 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 4 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 5 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 6 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 7 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 8 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 9 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 10 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 11 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 12 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 13 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 14 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 15 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 16 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 17 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 18 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 19 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 20 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 21 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 22 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 23 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 24 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 25 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 26 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 27 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 28 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 29 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 30 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 31 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 32 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 33 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 34 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 35 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 36 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 37 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 38 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 39 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 40 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 41 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 42 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 43 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 44 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 45 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 46 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 47 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 48 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 49 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 50 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 51 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 52 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 53 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 54 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 55 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 56 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 57 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 58 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 59 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 60 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 61 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 62 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 63 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 64 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 65 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 66 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 67 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 68 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 69 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 70 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 71 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 72 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 73 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 74 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 75 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 76 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 77 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 78 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 79 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 80 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 81 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 82 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 83 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 84 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 85 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 86 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 87 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 88 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 89 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 90 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 91 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 92 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 93 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 94 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 95 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 96 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 97 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 98 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 99 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 100 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 101 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 102 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 103 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 104 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 105 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 106 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 107 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 108 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 109 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 110 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 111 set forth in Table I, wherein said sequence comprises the polymorphic amino acid depicted. In another aspect, the present disclosure provides a MiHA peptide of the formula I or Ia as defined herein, wherein X¹ is a sequence comprising at least 8 amino acids of MiHA No. 112 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 113 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 114 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 115 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 116 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 117 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 118 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 119 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 120 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 121 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 122 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 123 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 124 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 125 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 126 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 127 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 128 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 129 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 130 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 131 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 132 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 133 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 134 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 135 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 136 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 137 set forth in Table I, wherein said sequence comprises the polymorphic amino acid depicted. In another aspect, the present disclosure provides a MiHA peptide of the formula I or Ia as defined herein, wherein X′ is a sequence comprising at least 8 amino acids of MiHA No. 138 set forth in Table I, wherein said sequence comprises the polymorphic amino acid depicted.

In an embodiment, the MiHA peptide is able to bind to, or to be presented by, HLA-A1 molecules (HLA-A*01:01 allele). In another aspect, the present disclosure provides an HLA-A1/HLA-A*01:01-binding MiHA peptide of 8-14 amino acids of the formula I as defined herein, wherein X¹ is a sequence of at least 8 amino acids of any one of the MiHA Nos. 5, 47, 81, 83, 85, 86, 90, 98, 105, 118, 119, 121, 122, 125 or 127, preferably MiHA Nos. 5, 47 and 81 set forth in Table I, wherein said sequence comprises the polymorphic amino acid depicted. In an embodiment, the HLA-A1/HLA-A*01:01-binding MiHA peptide comprises or consists of the sequence of MiHA Nos. 5, 47, 81, 83, 85, 86, 90, 98, 105, 118, 119, 121, 122, 125 or 127, preferably MiHA Nos. 5, 47 and 81. In an embodiment, the present disclosure provides a peptide pool or combination comprising the HLA-A1/HLA-A*01:01-binding MiHA peptides defined herein. In a further embodiment, the present disclosure provides a peptide pool or combination comprising all the HLA-A*01:01 binding MiHA peptides defined herein.

In an embodiment, the MiHA peptide is able to bind to, or to be presented by, HLA-A3 molecules (HLA-A*03:01 allele). In another aspect, the present disclosure provides an HLA-A3/HLA-A*03:01-binding MiHA peptide of 8-14 amino acids of the formula I as defined herein, wherein X¹ is a sequence of at least 8 amino acids of any one of the MiHA Nos. 36 and 77 set forth in Table I, wherein said sequence comprises the polymorphic amino acid depicted. In an embodiment, the HLA-A3/HLA-A*03:01-binding MiHA peptide comprises or consists of the sequence of MiHA Nos. 36 or 77. In an embodiment, the present disclosure provides a peptide pool or combination comprising the HLA-A3/HLA-A*03:01-binding MiHA peptides defined herein.

In an embodiment, the MiHA peptide is able to bind to, or to be presented by, HLA-A11 molecules (HLA-A*11:01 allele). In another aspect, the present disclosure provides an HLA-A11/HLA-A*11:01-binding MiHA peptide of 8-14 amino acids of the formula I as defined herein, wherein X¹ is a sequence of at least 8 amino acids of any one of the MiHA Nos. 1, 3, 13, 31, 61, 62, 69, 134 and 136, preferably MiHA Nos. 1, 3, 13, 31, 61, 62 and 69 set forth in Table I, wherein said sequence comprises the polymorphic amino acid depicted. In an embodiment, the HLA-A11/HLA-A*11:01-binding MiHA peptide comprises or consists of the sequence of MiHA Nos. 1, 3, 13, 31, 61, 62, 69, 134 and 136, preferably MiHA Nos. 1, 3, 13, 31, 61, 62 or 69. In an embodiment, the present disclosure provides a peptide pool or combination comprising two, three, four or more of the HLA-A11/HLA-A*11:01-binding MiHA peptides defined herein. In a further embodiment, the present disclosure provides a peptide pool or combination comprising all the HLA-A11/HLA-A*11:01-binding MiHA peptides defined herein.

In an embodiment, the MiHA peptide is able to bind to, or to be presented by, HLA-A24 molecules (HLA-A*24:02 allele). In another aspect, the present disclosure provides an HLA-A24/HLA-A*24:02-binding MiHA peptide of 8-14 amino acids of the formula I as defined herein, wherein X′ is a sequence of at least 8 amino acids of any one of the MiHA Nos. 33, 39, 40 and 79 set forth in Table I, wherein said sequence comprises the polymorphic amino acid depicted. In an embodiment, the HLA-A24/HLA-A*24:02-binding MiHA peptide comprises or consists of the sequence of MiHA Nos. 33, 39, 40 or 79. In an embodiment, the present disclosure provides a peptide pool or combination comprising two, three, four or more of the HLA-A24/HLA-A*24:02-binding MiHA peptides defined herein. In a further embodiment, the present disclosure provides a peptide pool or combination comprising all the HLA-A24/HLA-A*24:02-binding MiHA peptides defined herein.

In an embodiment, the MiHA peptide is able to bind to, or to be presented by, HLA-A29 molecules (HLA-A*29:02 allele). In another aspect, the present disclosure provides an HLA-A29/HLA-A*29:02-binding MiHA peptide of 8-14 amino acids of the formula I as defined herein, wherein X′ is a sequence of at least 8 amino acids of MiHA No. 21 set forth in Table I, wherein said sequence comprises the polymorphic amino acid depicted. In an embodiment, the HLA-A29/HLA-A*29:02-binding MiHA peptide comprises or consists of the sequence of MiHA No. 21.

In an embodiment, the MiHA peptide is able to bind to, or to be presented by, HLA-A32 molecules (HLA-A*32:01 allele). In another aspect, the present disclosure provides an HLA-A32/HLA-A*32:01-binding MiHA peptide of 8-14 amino acids of the formula I as defined herein, wherein X′ is a sequence of at least 8 amino acids of MiHA No. 55 set forth in Table I, wherein said sequence comprises the polymorphic amino acid depicted. In an embodiment, the HLA-A32/HLA-A*32:01-binding MiHA peptide comprises or consists of the sequence of MiHA No. 55.

In an embodiment, the MiHA peptide is able to bind to, or to be presented by, HLA-B7 molecules (HLA-B*07:02 allele). In another aspect, the present disclosure provides an HLA-B7/HLA-B*07:02-binding MiHA peptide of 8-14 amino acids of the formula I as defined herein, wherein X′ is a sequence of at least 8 amino acids of any one of the MiHA Nos. 8-12, 26, 28, 42, 43, 45, 46, 48, 49, 56-59, 65, 66, 70, 73, 74, 80, 82, 87-89, 91, 93-97, 99-103, 109-116, 120, 124, 126 and 128, preferably MiHA Nos. 8-12, 26, 28, 42, 43, 45, 46, 48, 49, 56-59, 65, 66, 70, 73, 74 and 80 set forth in Table I, wherein said sequence comprises the polymorphic amino acid depicted. In an embodiment, the HLA-B7/HLA-B*07:02-binding MiHA peptide comprises or consists of the sequence of MiHA Nos. 8-12, 26, 28, 42, 43, 45, 46, 48, 49, 56-59, 65, 66, 70, 73, 74, 80, 82, 87-89, 91, 93-97, 99-103, 109-116, 120, 124, 126 and 128, preferably MiHA Nos. 8-12, 26, 28, 42, 43, 45, 46, 48, 49, 56-59, 65, 66, 70, 73, 74 and 80. In an embodiment, the present disclosure provides a peptide pool or combination comprising two, three, four or more of the HLA-B7/HLA-B*07:02-binding MiHA peptides defined herein. In a further embodiment, the present disclosure provides a peptide pool or combination comprising all the HLA-B7/HLA-B*07:02-binding MiHA peptides defined herein.

In an embodiment, the MiHA peptide is able to bind to, or to be presented by, HLA-B8 molecules (HLA-B*08:01 allele). In another aspect, the present disclosure provides an HLA-B8/HLA-B*08:01-binding MiHA peptide of 8-14 amino acids of the formula I as defined herein, wherein X¹ is a sequence of at least 8 amino acids of any one of the MiHA Nos. 25, 27 and 71 set forth in Table I, wherein said sequence comprises the polymorphic amino acid depicted. In an embodiment, the HLA-B8/HLA-B*08:01-binding MiHA peptide comprises or consists of the sequence of MiHA Nos. 25, 27 or 71. In an embodiment, the present disclosure provides a peptide pool or combination comprising two, three, four or more of the HLA-B8/HLA-B*08:01-binding MiHA peptides defined herein. In a further embodiment, the present disclosure provides a peptide pool or combination comprising all the HLA-B8/HLA-B*08:01-binding MiHA peptides defined herein.

In an embodiment, the MiHA peptide is able to bind to, or to be presented by, HLA-B13 molecules (HLA-B*13:02 allele). In another aspect, the present disclosure provides an HLA-B13/HLA-B*13:02-binding MiHA peptide of 8-14 amino acids of the formula I as defined herein, wherein X¹ is a sequence of at least 8 amino acids of MiHA No. 67 set forth in Table I, wherein said sequence comprises the polymorphic amino acid depicted. In an embodiment, the HLA-B13/HLA-B*13:02-binding MiHA peptide comprises or consists of the sequence of MiHA No. 67.

In an embodiment, the MiHA peptide is able to bind to, or to be presented by, HLA-B14 molecules (HLA-B*14:02 allele). In another aspect, the present disclosure provides an HLA-B14/HLA-B*14:02-binding MiHA peptide of 8-14 amino acids of the formula I as defined herein, wherein X¹ is a sequence of at least 8 amino acids of any one of the MiHA Nos. 14, 15 and 44 set forth in Table I, wherein said sequence comprises the polymorphic amino acid depicted. In an embodiment, the HLA-B14/HLA-B*14:02-binding MiHA peptide comprises or consists of the sequence of MiHA Nos. 14, 15 or 44. In an embodiment, the present disclosure provides a peptide pool or combination comprising two, three, four or more of the HLA-B14/HLA-B*14:02-binding MiHA peptides defined herein. In a further embodiment, the present disclosure provides a peptide pool or combination comprising all the HLA-B14/HLA-B*14:02-binding MiHA peptides defined herein.

In an embodiment, the MiHA peptide is able to bind to, or to be presented by, HLA-B15 molecules (HLA-B*15:01 allele). In another aspect, the present disclosure provides an HLA-B15/HLA-B*15:01-binding MiHA peptide of 8-14 amino acids of the formula I as defined herein, wherein X¹ is a sequence of at least 8 amino acids of any one of the MiHA Nos. 38, 40, 72 and 76 set forth in Table I, wherein said sequence comprises the polymorphic amino acid depicted. In an embodiment, the HLA-B15/HLA-B*15:01-binding MiHA peptide comprises or consists of the sequence of MiHA Nos. 38, 40, 72 or 76. In an embodiment, the present disclosure provides a peptide pool or combination comprising two, three, four or more of the HLA-B15/HLA-B*15:01-binding MiHA peptides defined herein. In a further embodiment, the present disclosure provides a peptide pool or combination comprising all the HLA-B15/HLA-B*15:01-binding MiHA peptides defined herein.

In an embodiment, the MiHA peptide is able to bind to, or to be presented by, HLA-B18 molecules (HLA-B*18:01 allele). In another aspect, the present disclosure provides an HLA-B18/HLA-B*18:01-binding MiHA peptide of 8-14 amino acids of the formula I as defined herein, wherein X¹ is a sequence of at least 8 amino acids of any one of the MiHA Nos. 2, 20, 34, 41, 50, 52 and 54 set forth in Table I, wherein said sequence comprises the polymorphic amino acid depicted. In an embodiment, the HLA-B18/HLA-B*18:01-binding MiHA peptide comprises or consists of the sequence of MiHA Nos. 2, 20, 34, 41, 50, 52 or 54. In an embodiment, the present disclosure provides a peptide pool or combination comprising two, three, four or more of the HLA-B18/HLA-B*18:01-binding MiHA peptides defined herein. In a further embodiment, the present disclosure provides a peptide pool or combination comprising all the HLA-B18/HLA-B*18:01-binding MiHA peptides defined herein.

In an embodiment, the MiHA peptide is able to bind to, or to be presented by, HLA-B27 molecules (HLA-B*27:05 allele). In another aspect, the present disclosure provides an HLA-B27/HLA-B*27:05-binding MiHA peptide of 8-14 amino acids of the formula I as defined herein, wherein X¹ is a sequence of at least 8 amino acids of any one of the MiHA Nos. 1, 30, 32, 37, 65 and 68 set forth in Table I, wherein said sequence comprises the polymorphic amino acid depicted. In an embodiment, the HLA-B27/HLA-B*27:05-binding MiHA peptide comprises or consists of the sequence of MiHA Nos. 1, 30, 32, 37, 65 or 68. In an embodiment, the present disclosure provides a peptide pool or combination comprising two, three, four or more of the HLA-B27/HLA-B*27:05-binding MiHA peptides defined herein. In a further embodiment, the present disclosure provides a peptide pool or combination comprising all the HLA-B27/HLA-B*27:05-binding MiHA peptides defined herein.

In an embodiment, the MiHA peptide is able to bind to, or to be presented by, HLA-B35 molecules (HLA-B*35:01 allele). In another aspect, the present disclosure provides an HLA-B35/HLA-B*35:01-binding MiHA peptide of 8-14 amino acids of the formula I as defined herein, wherein X¹ is a sequence of at least 8 amino acids of MiHA No. 75 set forth in Table I, wherein said sequence comprises the polymorphic amino acid depicted. In an embodiment, the HLA-B35/HLA-B*35:01-binding MiHA peptide comprises or consists of the sequence of MiHA No. 75.

In an embodiment, the MiHA peptide is able to bind to, or to be presented by, HLA-B40 molecules (HLA-B*40:01 allele). In another aspect, the present disclosure provides an HLA-B40/HLA-B*40:01-binding MiHA peptide of 8-14 amino acids of the formula I as defined herein, wherein X¹ is a sequence of at least 8 amino acids of any one of the MiHA Nos. 2, 19, 21, 22, 29, 34, 35, 52, 64, 130, 131 and 133, preferably MiHA Nos. 2, 19, 21, 22, 29, 34, 35, 52 and 64 set forth in Table I, wherein said sequence comprises the polymorphic amino acid depicted. In an embodiment, the HLA-B40/HLA-B*40:01-binding MiHA peptide comprises or consists of the sequence of MiHA Nos. 2, 19, 21, 22, 29, 34, 35, 52, 64, 130, 131 and 133, preferably MiHA Nos. 2, 19, 21, 22, 29, 34, 35, 52 or 64. In an embodiment, the present disclosure provides a peptide pool or combination comprising two, three, four or more of the HLA-B40/HLA-B*40:01-binding MiHA peptides defined herein. In a further embodiment, the present disclosure provides a peptide pool or combination comprising all the HLA-B40/HLA-B*40:01-binding MiHA peptides defined herein.

In an embodiment, the MiHA peptide is able to bind to, or to be presented by, HLA-B44 molecules (HLA-B*44:02 or HLA-B*44:03 allele). In another aspect, the present disclosure provides an HLA-B44/HLA-B*44:02-binding MiHA peptide of 8-14 amino acids of the formula I as defined herein, wherein X¹ is a sequence of at least 8 amino acids of any one of the MiHA Nos. 2, 4, 6, 7, 16-24, 29, 34, 35, 50-53, 63, 64 and 78 set forth in Table I, wherein said sequence comprises the polymorphic amino acid depicted. In an embodiment, the HLA-B44/HLA-B*44:02-binding MiHA peptide comprises or consists of the sequence of MiHA Nos. 2, 4, 6, 7, 16-24, 29, 34, 35, 50-53, 63, 64 or 78. In an embodiment, the present disclosure provides a peptide pool or combination comprising two, three, four or more of the HLA-B44/HLA-B*44:02-binding MiHA peptides defined herein. In a further embodiment, the present disclosure provides a peptide pool or combination comprising all the HLA-B44/HLA-B*44:02-binding MiHA peptides defined herein. In another aspect, the present disclosure provides an HLA-B44/HLA-B*44:03-binding MiHA peptide of 8-14 amino acids of the formula I as defined herein, wherein X¹ is a sequence of at least 8 amino acids of any one of the MiHA Nos. 92, 106, 108, 117, 123, 129, 132, 135, 137 and 138 set forth in Table I, wherein said sequence comprises the polymorphic amino acid depicted. In an embodiment, the HLA-B44/HLA-B*44:03-binding MiHA peptide comprises or consists of the sequence of MiHA Nos. 92, 106, 108, 117, 123, 129, 132, 135, 137 and 138. In an embodiment, the present disclosure provides a peptide pool or combination comprising two, three, four or more of the HLA-B44/HLA-B*44:03-binding MiHA peptides defined herein. In a further embodiment, the present disclosure provides a peptide pool or combination comprising all the HLA-B44/HLA-B*44:03-binding MiHA peptides defined herein.

In an embodiment, the MiHA peptide is able to bind to, or to be presented by, HLA-B57 molecules (HLA-B*57:01 allele). In another aspect, the present disclosure provides an HLA-B57/HLA-B*57:01-binding MiHA peptide of 8-14 amino acids of the formula I as defined herein, wherein X¹ is a sequence of at least 8 amino acids of MiHA No. 34 set forth in Table I, wherein said sequence comprises the polymorphic amino acid depicted. In an embodiment, the HLA-B57/HLA-B*57:01-binding MiHA peptide comprises or consists of the sequence of MiHA No. 34.

In an embodiment, the MiHA peptide is able to bind to, or to be presented by, HLA-007 molecules (HLA-C*07:02 allele). In another aspect, the present disclosure provides an HLA-007/HLA-C*07:02-binding MiHA peptide of 8-14 amino acids of the formula I as defined herein, wherein X¹ is a sequence of at least 8 amino acids of MiHA No. 104 or 107 set forth in Table I, wherein said sequence comprises the polymorphic amino acid depicted. In an embodiment, the HLA-007/HLA-C*07:02-binding MiHA peptide comprises or consists of the sequence of MiHA No. 104 or 107. In an embodiment, the present disclosure provides a peptide pool or combination comprising the HLA-007/HLA-C*07:02-binding MiHA peptides defined herein.

In an embodiment, the MiHA peptide is derived from a gene that does not exhibit ubiquitous expression. The expression “does not exhibit ubiquitous expression” is used herein to refer to a gene which, according to the data from Fagerberg et al., Mol Cell Proteomics 2014 13: 397-406, is not expressed with a FPKM >10 in all 27 tissues disclosed therein.

In an embodiment, the MiHA peptide derives from a locus with a minor allele frequency (MAF) of at least 0.05 as determined according to data from the dbSNP database (NCBI) and the National Heart, Lung and Blood Institute (NHLBI) Exome Sequencing Project (ESP) (as set forth in Table II). In an embodiment, the MiHA peptide derives from a locus with a MAF of at least 0.1 as determined according to data from the dbSNP database (NCBI) and/or the NHLBI Exome Sequencing Project (ESP). In an embodiment, the MiHA peptide derives from a locus with a MAF of at least 0.1 as determined according to data from the dbSNP database (NCBI) and the NHLBI Exome Sequencing Project (ESP). In an embodiment, the MiHA peptide derives from a locus with a MAF of at least 0.15 as determined according to data from the dbSNP database (NCBI) and/or the NHLBI Exome Sequencing Project (ESP). In an embodiment, the MiHA peptide derives from a locus with a MAF of at least 0.15 as determined according to data from the dbSNP database (NCBI) and the NHLBI Exome Sequencing Project (ESP). In an embodiment, the MiHA peptide derives from a locus with a MAF of at least 0.2 as determined according to data from the dbSNP database (NCBI) and/or the NHLBI Exome Sequencing Project (ESP). In an embodiment, the MiHA peptide derives from a locus with a MAF of at least 0.2 as determined according to data from the dbSNP database (NCBI) and the NHLBI Exome Sequencing Project (ESP). In an embodiment, the MiHA peptide derives from a locus with a MAF of at least 0.25 as determined according to data from the dbSNP database (NCBI) and/or the NHLBI Exome Sequencing Project (ESP). In an embodiment, the MiHA peptide derives from a locus with a MAF of at least 0.25 as determined according to data from the dbSNP database (NCBI) and the NHLBI Exome Sequencing Project (ESP). In an embodiment, the MiHA peptide derives from a locus with a MAF of at least 0.3 as determined according to data from the dbSNP database (NCBI) and/or the NHLBI Exome Sequencing Project (ESP). In an embodiment, the MiHA peptide derives from a locus with a MAF of at least 0.3 as determined according to data from the dbSNP database (NCBI) and the NHLBI Exome Sequencing Project (ESP). In an embodiment, the MiHA peptide derives from a locus with a MAF of at least 0.35 as determined according to data from the dbSNP database (NCBI) and/or the NHLBI Exome Sequencing Project (ESP). In an embodiment, the MiHA peptide derives from a locus with a MAF of at least 0.35 as determined according to data from the dbSNP database (NCBI) and the NHLBI Exome Sequencing Project (ESP). In an embodiment, the MiHA peptide derives from a locus with a MAF of at least 0.4 as determined according to data from the dbSNP database (NCBI) and/or the NHLBI Exome Sequencing Project (ESP). In an embodiment, the MiHA peptide derives from a locus with a MAF of at least 0.4 as determined according to data from the dbSNP database (NCBI) and the NHLBI Exome Sequencing Project (ESP).

In some embodiments, the present disclosure provides a MiHA peptide comprising any combination/subcombination of the features or properties defined herein, for example, a MiHA peptide of the formula I as defined herein, wherein the peptide (i) binds to HLA-A2 molecules, (ii) derives from a gene that does not exhibit ubiquitous expression and (iii) derives from a locus with a MAF of at least 0.1 as determined according to data from the dbSNP database (NCBI) and/or the NHLBI Exome Sequencing Project (ESP).

In general, peptides presented in the context of HLA class I vary in length from about 7 or 8 to about 15, or preferably 8 to 14 amino acid residues. In some embodiments of the methods of the disclosure, longer peptides comprising the MiHA peptide sequences defined herein are artificially loaded into cells such as antigen presenting cells (APCs), processed by the cells and the MiHA peptide is presented by MHC class I molecules at the surface of the APC. In this method, peptides/polypeptides longer than 15 amino acid residues (i.e. a MiHA precursor peptide, such as those defined by formula Ia herein) can be loaded into APCs, are processed by proteases in the APC cytosol providing the corresponding MiHA peptide as defined herein for presentation. In some embodiments, the precursor peptide/polypeptide (e.g., polypeptide of formula Ia defined herein) that is used to generate the MiHA peptide defined herein is for example 1000, 500, 400, 300, 200, 150, 100, 75, 50, 45, 40, 35, 30, 25, 20 or 15 amino acids or less. Thus, all the methods and processes using the MiHA peptides described herein include the use of longer peptides or polypeptides (including the native protein), i.e. MiHA precursor peptides/polypeptides, to induce the presentation of the “final” 8-14 MiHA peptide following processing by the cell (APCs). In some embodiments, the herein-mentioned MiHA peptide is about 8 to 14, 8 to 13, or 8 to 12 amino acids long (e.g., 8, 9, 10, 11, 12 or 13 amino acids long), small enough for a direct fit in an HLA class I molecule (e.g., HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule), but it may also be larger, between 12 to about 20, 25, 30, 35, 40, 45 or 50 amino acids, and a MiHA peptide corresponding to the domain defined by X¹ herein be presented by HLA molecules only after cellular uptake and intracellular processing by the proteasome and/or other proteases and transport before presentation in the groove of an HLA class I molecule (HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule), as explained herein. In an embodiment, the MiHA peptide consists of an amino acid sequence of 8 to 14 amino acids, e.g., 8, 9, 10, 11, 12, 13, or 14 amino acids, wherein the sequence is the sequence of any one of MiHAs Nos: 1-138 or MiHAs Nos: 1-81 set forth in Table I. In another aspect, the present disclosure provides a MiHA peptide consisting of an amino acid sequence of 8 to 14 amino acids, e.g., 8, 9, 10, 11, 12, 13, or 14 amino acids, said amino acid sequence consisting of the sequence of MiHAs Nos: 1-138 or MiHAs Nos: 1-81, preferably MiHA Nos. 3, 5, 8-15, 25-28, 30-33, 36-49, 54-61, 65-66, 68-77 and 79-81. In an embodiment, the at least 8 amino acids of one of MiHA Nos. MiHAs Nos: 1-138 or MiHAs Nos: 1-81, preferably MiHA Nos. 3, 5, 8-15, 25-28, 30-33, 36-49, 54-61, 65-66, 68-77 and 79-81 are contiguous amino acids. In an embodiment, X¹ is a domain comprising at least 8 amino acids of any one of MiHAs Nos: 1-138 or MiHAs Nos: 1-81, preferably MiHA Nos. 3, 5, 8-15, 25-28, 30-33, 36-49, 54-61, 65-66, 68-77 and 79-81, wherein said sequence comprises the polymorphic amino acid depicted. In another embodiment, X¹ is a sequence comprising, or consisting of, the amino acids of any one of MiHAs Nos: 1-138 or MiHAs Nos: 1-81, preferably MiHA Nos. 3, 5, 8-15, 25-28, 30-33, 36-49, 54-61, 65-66, 68-77 and 79-81.

The term “amino acid” as used herein includes both L- and D-isomers of the naturally occurring amino acids as well as other amino acids (e.g., naturally-occurring amino acids, non-naturally-occurring amino acids, amino acids which are not encoded by nucleic acid sequences, etc.) used in peptide chemistry to prepare synthetic analogs of MiHA peptides. Examples of naturally occurring amino acids are glycine, alanine, valine, leucine, isoleucine, serine, threonine, etc. Other amino acids include for example non-genetically encoded forms of amino acids, as well as a conservative substitution of an L-amino acid. Naturally-occurring non-genetically encoded amino acids include, for example, beta-alanine, 3-amino-propionic acid, 2,3-diaminopropionic acid, alpha-aminoisobutyric acid (Aib), 4-amino-butyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, norleucine (Nle), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (Hoarg), N-acetyl lysine, 2-amino butyric acid, 2-amino butyric acid, 2,4,-diaminobutyric acid (D- or L-), p-aminophenylalanine, N-methylvaline, homocysteine, homoserine (HoSer), cysteic acid, epsilon-amino hexanoic acid, delta-amino valeric acid, or 2,3-diaminobutyric acid (D- or L-), etc. These amino acids are well known in the art of biochemistry/peptide chemistry. In an embodiment, the MiHA peptide comprises only naturally-occurring amino acids.

In embodiments, the MiHA peptides described herein include peptides with altered sequences containing substitutions of functionally equivalent amino acid residues, relative to the herein-mentioned sequences. For example, one or more amino acid residues within the sequence can be substituted by another amino acid of a similar polarity (having similar physico-chemical properties) which acts as a functional equivalent, resulting in a silent alteration. Substitution for an amino acid within the sequence may be selected from other members of the class to which the amino acid belongs. For example, positively charged (basic) amino acids include arginine, lysine and histidine (as well as homoarginine and ornithine). Nonpolar (hydrophobic) amino acids include leucine, isoleucine, alanine, phenylalanine, valine, proline, tryptophan and methionine. Uncharged polar amino acids include serine, threonine, cysteine, tyrosine, asparagine and glutamine. Negatively charged (acidic) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. The herein-mentioned MiHA peptide may comprise all L-amino acids, all D-amino acids or a mixture of L- and D-amino acids. In an embodiment, the herein-mentioned MiHA peptide comprises all L-amino acids.

In an embodiment, in the sequences of the MiHA peptides comprising one of sequences of MiHAs Nos: 1-138 or MiHAs Nos: 1-81, the amino acid residues that do not substantially contribute to interactions with the T-cell receptor may be modified by replacement with other amino acid whose incorporation does not substantially affect T-cell reactivity and does not eliminate binding to the relevant MHC.

The MiHA peptide may also be N- and/or C-terminally capped or modified to prevent degradation, increase stability, affinity and/or uptake. In an embodiment, the amino terminal residue (i.e., the free amino group at the N-terminal end) of the MiHA peptide is modified (e.g., for protection against degradation), for example by covalent attachment of a moiety/chemical group (Z¹). Z¹ may be a straight chained or branched alkyl group of one to eight carbons, or an acyl group (R—CO—), wherein R is a hydrophobic moiety (e.g., acetyl, propionyl, butanyl, iso-propionyl, or iso-butanyl), or an aroyl group (Ar—CO—), wherein Ar is an aryl group. In an embodiment, the acyl group is a C₁-C₁₆ or C₃-C₁₆ acyl group (linear or branched, saturated or unsaturated), in a further embodiment, a saturated C₁-C₆ acyl group (linear or branched) or an unsaturated C₃-C₆ acyl group (linear or branched), for example an acetyl group (CH₃—CO—, Ac). In an embodiment, Z¹ is absent. The carboxy terminal residue (i.e., the free carboxy group at the C-terminal end of the MiHA peptide) of the MiHA peptide may be modified (e.g., for protection against degradation), for example by amidation (replacement of the OH group by a NH₂ group), thus in such a case Z² is a NH₂ group. In an embodiment, Z² may be an hydroxamate group, a nitrile group, an amide (primary, secondary or tertiary) group, an aliphatic amine of one to ten carbons such as methyl amine, iso-butylamine, iso-valerylamine or cyclohexylamine, an aromatic or arylalkyl amine such as aniline, napthylamine, benzylamine, cinnamylamine, or phenylethylamine, an alcohol or CH₂OH. In an embodiment, Z² is absent. In an embodiment, the MiHA peptide comprises one of sequences Nos. 1-138 or 1-81, preferably MiHA Nos. 3, 5, 8-15, 25-28, 30-33, 36-49, 54-61, 65-66, 68-77 and 79-81 set forth in Table I. In an embodiment, the MiHA peptide consists of one of sequences Nos. 1-138 or 1-81, preferably MiHA Nos. 3, 5, 8-15, 25-28, 30-33, 36-49, 54-61, 65-66, 68-77 and 79-81 set forth in Table I, i.e. wherein Z¹ and Z² are absent.

The MiHA peptides of the disclosure may be produced by expression in a host cell comprising a nucleic acid encoding the MiHA peptides (recombinant expression) or by chemical synthesis (e.g., solid-phase peptide synthesis). Peptides can be readily synthesized by manual and/or automated solid phase procedures well known in the art. Suitable syntheses can be performed for example by utilizing “T-boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the MiHA peptides may be prepared by way of segment condensation, as described, for example, in Liu et al., Tetrahedron Lett. 37: 933-936, 1996; Baca et al., J. Am. Chem. Soc. 117: 1881-1887, 1995; Tam et al., Int. J. Peptide Protein Res. 45: 209-216, 1995; Schnolzer and Kent, Science 256: 221-225, 1992; Liu and Tam, J. Am. Chem. Soc. 116: 4149-4153, 1994; Liu and Tam, Proc. Natl. Acad. Sci. USA 91: 6584-6588, 1994; and Yamashiro and Li, Int. J. Peptide Protein Res. 31: 322-334, 1988). Other methods useful for synthesizing the MiHA peptides are described in Nakagawa et al., J. Am. Chem. Soc. 107: 7087-7092, 1985. In an embodiment, the MiHA peptide of the formula I or Ia is chemically synthesized (synthetic peptide). Another embodiment of the present disclosure relates to a non-naturally occurring peptide wherein said peptide consists or consists essentially of an amino acid sequences defined herein and has been synthetically produced (e.g. synthesized) as a pharmaceutically acceptable salt. The salts of the peptides according to the present disclosure differ substantially from the peptides in their state(s) in vivo, as the peptides as generated in vivo are no salts. The non-natural salt form of the peptide may modulate the solubility of the peptide, in particular in the context of pharmaceutical compositions comprising the peptides, e.g. the peptide vaccines as disclosed herein. Preferably, the salts are pharmaceutically acceptable salts of the peptides.

In an embodiment, the herein-mentioned MiHA peptide is substantially pure. A compound is “substantially pure” when it is separated from the components that naturally accompany it. Typically, a compound is substantially pure when it is at least 60%, more generally 75%, 80% or 85%, preferably over 90% and more preferably over 95%, by weight, of the total material in a sample. Thus, for example, a polypeptide that is chemically synthesized or produced by recombinant technology will generally be substantially free from its naturally associated components, e.g. components of its source macromolecule. A nucleic acid molecule is substantially pure when it is not immediately contiguous with (i.e., covalently linked to) the coding sequences with which it is normally contiguous in the naturally occurring genome of the organism from which the nucleic acid is derived. A substantially pure compound can be obtained, for example, by extraction from a natural source; by expression of a recombinant nucleic acid molecule encoding a peptide compound; or by chemical synthesis. Purity can be measured using any appropriate method such as column chromatography, gel electrophoresis, HPLC, etc. In an embodiment, the MiHA peptide is in solution. In another embodiment, the MiHA peptide is in solid form, e.g., lyophilized.

In another aspect, the disclosure further provides a nucleic acid (isolated) encoding the herein-mentioned MiHA peptides or a MiHA precursor-peptide. In an embodiment, the nucleic acid comprises from about 21 nucleotides to about 45 nucleotides, from about 24 to about 45 nucleotides, for example 24, 27, 30, 33, 36, 39, 42 or 45 nucleotides. “Isolated”, as used herein, refers to a peptide or nucleic molecule separated from other components that are present in the natural environment of the molecule or a naturally occurring source macromolecule (e.g., including other nucleic acids, proteins, lipids, sugars, etc.). “Synthetic”, as used herein, refers to a peptide or nucleic molecule that is not isolated from its natural sources, e.g., which is produced through recombinant technology or using chemical synthesis. A nucleic acid of the disclosure may be used for recombinant expression of the MiHA peptide of the disclosure, and may be included in a vector or plasmid, such as a cloning vector or an expression vector, which may be transfected into a host cell. In an embodiment, the disclosure provides a cloning or expression vector or plasmid comprising a nucleic acid sequence encoding the MiHA peptide of the disclosure. Alternatively, a nucleic acid encoding a MiHA peptide of the disclosure may be incorporated into the genome of the host cell. In either case, the host cell expresses the MiHA peptide or protein encoded by the nucleic acid. The term “host cell” as used herein refers not only to the particular subject cell, but to the progeny or potential progeny of such a cell. A host cell can be any prokaryotic (e.g., E. coli) or eukaryotic cell (e.g., insect cells, yeast or mammalian cells) capable of expressing the MiHA peptides described herein. The vector or plasmid contains the necessary elements for the transcription and translation of the inserted coding sequence, and may contain other components such as resistance genes, cloning sites, etc. Methods that are well known to those skilled in the art may be used to construct expression vectors containing sequences encoding peptides or polypeptides and appropriate transcriptional and translational control/regulatory elements operably linked thereto. These methods include in vitro recombinant DNA techniques, synthetic techniques, and in vivo genetic recombination. Such techniques are described in Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y. “Operably linked” refers to a juxtaposition of components, particularly nucleotide sequences, such that the normal function of the components can be performed. Thus, a coding sequence that is operably linked to regulatory sequences refers to a configuration of nucleotide sequences wherein the coding sequences can be expressed under the regulatory control, that is, transcriptional and/or translational control, of the regulatory sequences. “Regulatory/control region” or “regulatory/control sequence”, as used herein, refers to the non-coding nucleotide sequences that are involved in the regulation of the expression of a coding nucleic acid. Thus, the term regulatory region includes promoter sequences, regulatory protein binding sites, upstream activator sequences, and the like.

In another aspect, the present disclosure provides a MHC class I molecule comprising (i.e. presenting or bound to) a MiHA peptide. In an embodiment, the MHC class I molecule is a HLA-A1 molecule, in a further embodiment a HLA-A*01:01 molecule. In an embodiment, the MHC class I molecule is a HLA-A3 molecule, in a further embodiment a HLA-A*03:01 molecule. In an embodiment, the MHC class I molecule is a HLA-A11 molecule, in a further embodiment a HLA-A*11:01 molecule. In an embodiment, the MHC class I molecule is a HLA-A24 molecule, in a further embodiment a HLA-A*24:02 molecule. In an embodiment, the MHC class I molecule is a HLA-A29 molecule, in a further embodiment a HLA-A*29:02 molecule. In an embodiment, the MHC class I molecule is a HLA-A32 molecule, in a further embodiment a HLA-A*32:01 molecule. In another embodiment, the MHC class I molecule is a HLA-B44 molecule, in a further embodiment a HLA-B*44:02 or HLA-B*44:03 molecule. In another embodiment, the MHC class I molecule is a HLA-B7 molecule, in a further embodiment a HLA-B*07:02 molecule. In another embodiment, the MHC class I molecule is a HLA-B8 molecule, in a further embodiment a HLA-B*08:01 molecule. In another embodiment, the MHC class I molecule is a HLA-B13 molecule, in a further embodiment a HLA-B*13:02 molecule. In another embodiment, the MHC class I molecule is a HLA-B14 molecule, in a further embodiment a HLA-B*14:02 molecule. In another embodiment, the MHC class I molecule is a HLA-B15 molecule, in a further embodiment a HLA-B*15:01 molecule. In another embodiment, the MHC class I molecule is a HLA-B18 molecule, in a further embodiment a HLA-B*18:01 molecule. In another embodiment, the MHC class I molecule is a HLA-B27 molecule, in a further embodiment a HLA-B*27:05 molecule. In another embodiment, the MHC class I molecule is a HLA-B35 molecule, in a further embodiment a HLA-B*35:01 molecule. In another embodiment, the MHC class I molecule is a HLA-B40 molecule, in a further embodiment a HLA-B*40:01 molecule. In another embodiment, the MHC class I molecule is a HLA-007 molecule, in a further embodiment a HLA-C*07:02 molecule. In an embodiment, the MiHA peptide is non-covalently bound to the MHC class I molecule (i.e., the MiHA peptide is loaded into, or non-covalently bound to the peptide binding groove/pocket of the MHC class I molecule). In another embodiment, the MiHA peptide is covalently attached/bound to the MHC class I molecule (alpha chain). In such a construct, the MiHA peptide and the MHC class I molecule (alpha chain) are produced as a synthetic fusion protein, typically with a short (e.g., 5 to 20 residues, preferably about 8-12, e.g., 10) flexible linker or spacer (e.g., a polyglycine linker). In another aspect, the disclosure provides a nucleic acid encoding a fusion protein comprising a MiHA peptide defined herein fused to a MHC class I molecule (alpha chain). In an embodiment, the MHC class I molecule (alpha chain)—peptide complex is multimerized. Accordingly, in another aspect, the present disclosure provides a multimer of MHC class I molecule loaded (covalently or not) with the herein-mentioned MiHA peptide. Such multimers may be attached to a tag, for example a fluorescent tag, which allows the detection of the multimers. A great number of strategies have been developed for the production of MHC multimers, including MHC dimers, tetramers, pentamers, octamers, etc. (reviewed in Bakker and Schumacher, Current Opinion in Immunology 2005, 17:428-433). MHC multimers are useful, for example, for the detection and purification of antigen-specific T cells. Thus, in another aspect, the present disclosure provides a method for detecting or purifying (isolating, enriching) CD8⁺ T lymphocytes specific for a MiHA peptide defined herein, the method comprising contacting a cell population with a multimer of MHC class I molecule loaded (covalently or not) with the MiHA peptide; and detecting or isolating the CD8⁺ T lymphocytes bound by the MHC class I multimers. CD8⁺ T lymphocytes bound by the MHC class I multimers may be isolated using known methods, for example fluorescence activated cell sorting (FACS) or magnetic activated cell sorting (MACS).

In yet another aspect, the present disclosure provides a cell (e.g., a host cell), in an embodiment an isolated cell, comprising the herein-mentioned nucleic acid, vector or plasmid of the disclosure, i.e. a nucleic acid or vector encoding one or more MiHA peptides. In another aspect, the present disclosure provides a cell expressing at its surface a MHC class I molecule (e.g., a HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule) bound to or presenting a MiHA peptide according to the disclosure. In one embodiment, the host cell is an eukaryotic cell, such as a mammalian cell, preferably a human cell. a cell line or an immortalized cell. In another embodiment, the cell is an antigen-presenting cell (APC). In one embodiment, the host cell is a primary cell, a cell line or an immortalized cell. In another embodiment, the cell is an antigen-presenting cell (APC). Nucleic acids and vectors can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), and other laboratory manuals. Methods for introducing nucleic acids into mammalian cells in vivo are also known, and may be used to deliver the vector DNA of the disclosure to a subject for gene therapy.

Cells such as APCs can be loaded with one or more MiHA peptides using a variety of methods known in the art. As used herein “loading a cell” with a MiHA peptide means that RNA or DNA encoding the MiHA peptide, or the MiHA peptide, is transfected into the cells or alternatively that the APC is transformed with a nucleic acid encoding the MiHA peptide. The cell can also be loaded by contacting the cell with exogenous MiHA peptides that can bind directly to MHC class I molecule present at the cell surface (e.g., peptide-pulsed cells). The MiHA peptides may also be fused to a domain or motif that facilitates its presentation by MHC class I molecules, for example to an endoplasmic reticulum (ER) retrieval signal, a C-terminal Lys-Asp-Glu-Leu sequence (see Wang et al., Eur J Immunol. 2004 December; 34(12):3582-94).

### Compositions

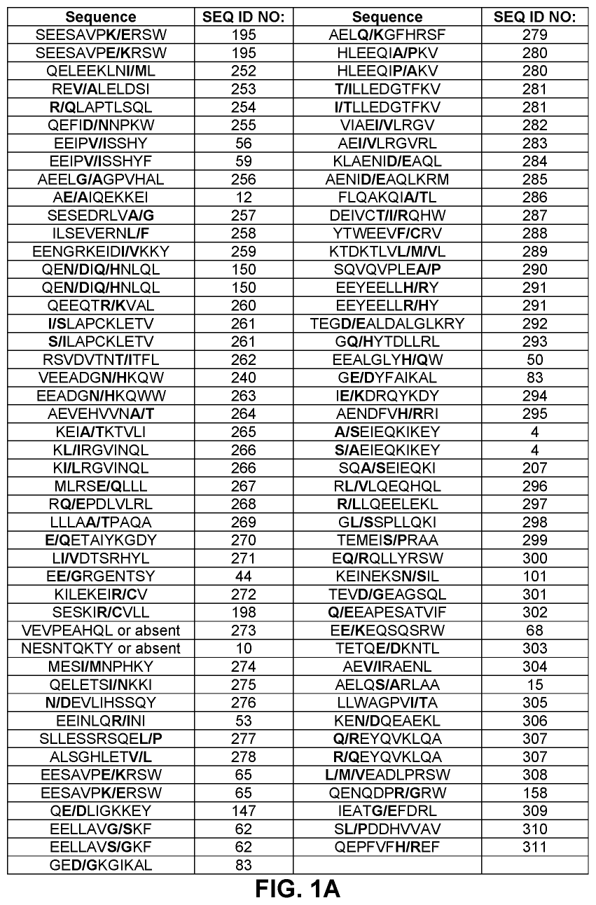

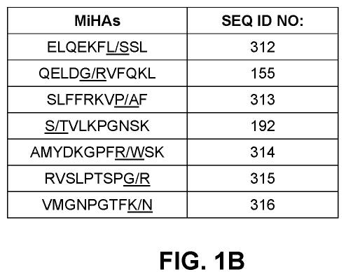

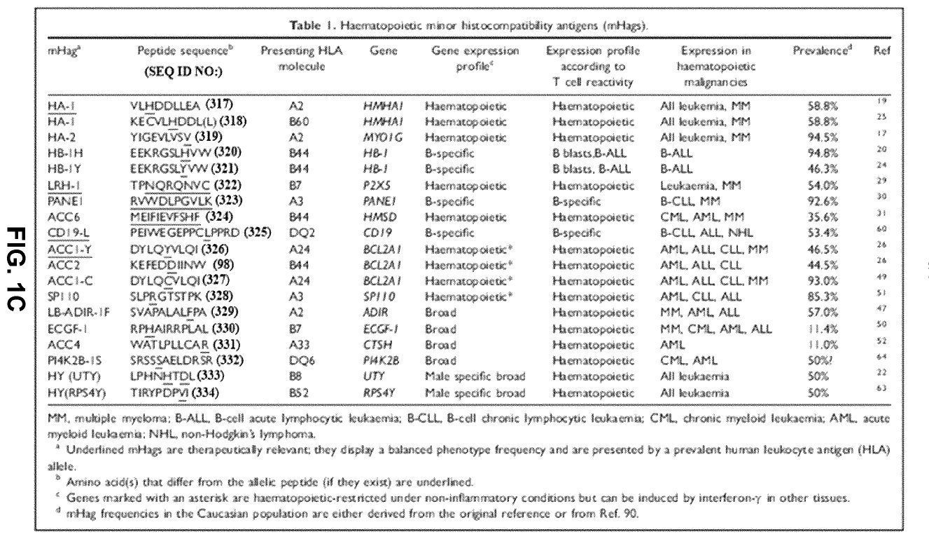

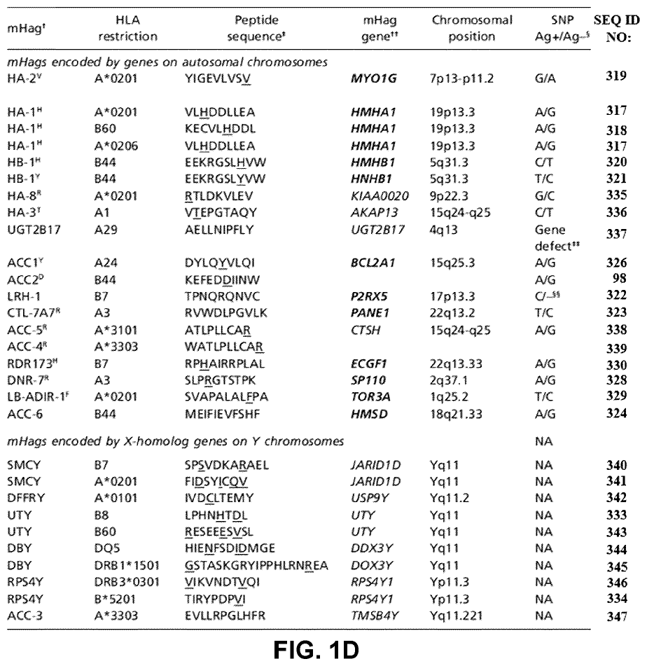

In another aspect, the present disclosure provides a composition or peptide combination/pool comprising any one of, or any combination of, the MiHA peptides defined herein (or a nucleic acid encoding said peptide(s)). In an embodiment, the composition comprises any combination of the MiHA peptides defined herein (e.g., any combination of MiHAs Nos. 1-138 or 1-81, preferably MiHA Nos. 3, 5, 8-15, 25-28, 30-33, 36-49, 54-61, 65-66, 68-77 and 79-81 set forth in Table I), or a combination of nucleic acids encoding said MiHA peptides). For example, the composition may comprise a first MiHA peptide which correspond to MiHA No. 1 and a second MiHA peptide that corresponds to MiHA No. 2. Compositions comprising any combination/sub-combination of the MiHA peptides defined herein are encompassed by the present disclosure. In another embodiment, the combination or pool may comprise one or more known MiHAs, such as the MiHAs disclosed in PCT publications Nos. WO/2016/127249 and WO/2014/026277, in Spaapen and Mutis, Best Practice & Research Clinical Hematology, 21(3): 543-557 and in Akatsuka et al., Cancer Sci, 98(8): 1139-1146, 2007 (see FIGS. 1A-1D). In an embodiment, the composition or peptide combination/pool comprises at least 2, 3, 4, 5, 6, 7, 8, 9, or 10 MiHA peptides, wherein at least one of said MiHA peptide comprising the MiHAs Nos. 1-138 or 1-81, preferably MiHA Nos. 3, 5, 8-15, 25-28, 30-33, 36-49, 54-61, 65-66, 68-77 and 79-81. In an embodiment, the composition or peptide combination/pool comprises at least 2, 3, 4, 5, 6, 7, 8, 9, or 10 MiHA peptides binding to the same MHC class I molecule (e.g., HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule). In a further embodiment, a MHC class I molecule (HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule) that presents a MiHA peptide is expressed at the surface of a cell, e.g., an APC. In an embodiment, the disclosure provides an APC loaded with one or more MiHA peptides bound to MHC class I molecules. In yet a further embodiment, the disclosure provides an isolated MHC class I/MiHA peptide complex.

Thus, in another aspect, the present disclosure provides a composition comprising any one of, or any combination of, the MiHA peptides defined herein and a cell expressing a MHC class I molecule (HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule). APC for use in the present disclosure are not limited to a particular type of cell and include professional APCs such as dendritic cells (DCs), Langerhans cells, macrophages and B cells, which are known to present proteinaceous antigens on their cell surface so as to be recognized by CD8⁺ T lymphocytes. For example, an APC can be obtained by inducing DCs from peripheral blood monocytes and then contacting (stimulating) the MiHA peptides, either in vitro, ex vivo or in vivo. APC can also be activated to present a MiHA peptide in vivo where one or more of the MiHA peptides of the disclosure are administered to a subject and APCs that present a MiHA peptide are induced in the body of the subject. The phrase “inducing an ARC” or “stimulating an ARC” includes contacting or loading a cell with one or more MiHA peptides, or nucleic acids encoding the MiHA peptides such that the MiHA peptides are presented at its surface by MHC class I molecules (e.g., HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule). As noted herein, according to the present disclosure, the MiHA peptides may be loaded indirectly for example using longer peptides/polypeptides comprising the sequence of the MiHAs (including the native protein), which is then processed (e.g., by proteases) inside the APCs to generate the MiHA peptide/MHC class I complexes at the surface of the cells. After loading APCs with MiHA peptides and allowing the APCs to present the MiHA peptides, the APCs can be administered to a subject as a vaccine. For example, the ex vivo administration can include the steps of: (a) collecting APCs from a first subject, (b) contacting/loading the APCs of step (a) with a MiHA peptide to form MHC class I/MiHA peptide complexes at the surface of the APCs; and (c) administering the peptide-loaded APCs to a second subject in need for treatment.

The first subject and the second subject can be the same subject (e.g., autologous vaccine), or may be different subjects (e.g., allogeneic vaccine). Alternatively, according to the present disclosure, use of a MiHA peptide described herein (or a combination thereof) for manufacturing a composition (e.g., a pharmaceutical composition) for inducing antigen-presenting cells is provided. In addition, the present disclosure provides a method or process for manufacturing a pharmaceutical composition for inducing antigen-presenting cells, wherein the method or the process includes the step of admixing or formulating the MiHA peptide, or a combination thereof, with a pharmaceutically acceptable carrier. Cells such as APCs expressing a MHC class I molecule (e.g., HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule) loaded with any one of, or any combination of, the MiHA peptides defined herein, may be used for stimulating/amplifying CD8⁺ T lymphocytes, for example autologous CD8⁺ T lymphocytes. Accordingly, in another aspect, the present disclosure provides a composition comprising any one of, or any combination of, the MiHA peptides defined herein (or a nucleic acid or vector encoding same); a cell expressing a MHC class I molecule (e.g., HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 and/or HLA-B57 molecule) and a T lymphocyte, more specifically a CD8⁺ T lymphocyte (e.g., a population of cells comprising CD8⁺ T lymphocytes).

In an embodiment, the composition further comprises a buffer, an excipient, a carrier, a diluent and/or a medium (e.g., a culture medium). In a further embodiment, the buffer, excipient, carrier, diluent and/or medium is/are pharmaceutically acceptable buffer(s), excipient(s), carrier(s), diluent(s) and/or medium (media). As used herein “pharmaceutically acceptable buffer, excipient, carrier, diluent and/or medium” includes any and all solvents, buffers, binders, lubricants, fillers, thickening agents, disintegrants, plasticizers, coatings, barrier layer formulations, lubricants, stabilizing agent, release-delaying agents, dispersion media, coatings, antibacterial and antifungal agents, isotonic agents, and the like that are physiologically compatible, do not interfere with effectiveness of the biological activity of the active ingredient(s) and that are not toxic to the subject. The use of such media and agents for pharmaceutically active substances is well known in the art (Rowe et al., Handbook of pharmaceutical excipients, 2003, 4th edition, Pharmaceutical Press, London UK). Except insofar as any conventional media or agent is incompatible with the active compound (peptides, cells), use thereof in the compositions of the disclosure is contemplated. In an embodiment, the buffer, excipient, carrier and/or medium is a non-naturally occurring buffer, excipient, carrier and/or medium.

In another aspect, the present disclosure provides a composition comprising one of more of the any one of, or any combination of, the MiHA peptides defined herein (or a nucleic acid encoding said peptide(s)), and a buffer, an excipient, a carrier, a diluent and/or a medium. For compositions comprising cells (e.g., APCs, T lymphocytes), the composition comprises a suitable medium that allows the maintenance of viable cells. Representative examples of such media include saline solution, Earl's Balanced Salt Solution (Life Technologies®) or Plasmalyte® (Baxter International®). In an embodiment, the composition (e.g., pharmaceutical composition) is an “immunogenic composition”, “vaccine composition” or “vaccine”. The term “Immunogenic composition”, “vaccine composition” or “vaccine” as used herein refers to a composition or formulation comprising one or more MiHA peptides or vaccine vector and which is capable of inducing an immune response against the one or more MiHA peptides present therein when administered to a subject. Vaccination methods for inducing an immune response in a mammal comprise use of a vaccine or vaccine vector to be administered by any conventional route known in the vaccine field, e.g., via a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g., via a transdermal delivery system such as a patch). In an embodiment, the MiHA peptide (or a combination thereof) is conjugated to a carrier protein (conjugate vaccine) to increase the immunogenicity of the MiHA peptide(s). The present disclosure thus provides a composition (conjugate) comprising a MiHA peptide (or a combination thereof) and a carrier protein. For example, the MiHA peptide(s) may be conjugated to a Toll-like receptor (TLR) ligand (see, e.g., Zom et al., Adv Immunol. 2012, 114: 177-201) or polymers/dendrimers (see, e.g., Liu et al., Biomacromolecules. 2013 Aug. 12; 14(8):2798-806). In an embodiment, the immunogenic composition or vaccine further comprises an adjuvant. “Adjuvant” refers to a substance which, when added to an immunogenic agent such as an antigen (MiHA peptides and/or cells according to the present disclosure), nonspecifically enhances or potentiates an immune response to the agent in the host upon exposure to the mixture. Examples of adjuvants currently used in the field of vaccines include (1) mineral salts (aluminum salts such as aluminum phosphate and aluminum hydroxide, calcium phosphate gels), squalene, (2) oil-based adjuvants such as oil emulsions and surfactant based formulations, e.g., MF59 (microfluidised detergent stabilised oil-in-water emulsion), QS21 (purified saponin), AS02 [SBAS2] (oil-in-water emulsion+MPL+QS-21), (3) particulate adjuvants, e.g., virosomes (unilamellar liposomal vehicles incorporating influenza haemagglutinin), AS04 ([SBAS4] aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL+M. Phlei cell wall skeleton), AGP [RC-529] (synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified LT and CT (genetically modified bacterial toxins to provide non-toxic adjuvant effects), (5) endogenous human immunomodulators, e.g., hGM-CSF or hIL-12 (cytokines that can be administered either as protein or plasmid encoded), Immudaptin (C3d tandem array) and/or (6) inert vehicles, such as gold particles, and the like.

In an embodiment, the MiHA peptide(s) or composition comprising same is/are in lyophilized form. In another embodiment, the MiHA peptide(s) or composition comprising same is/are in a liquid composition. In a further embodiment, the MiHA peptide(s) is/are at a concentration of about 0.01 μg/mL to about 100 μg/mL in the composition. In further embodiments, the MiHA peptide(s) is/are at a concentration of about 0.2 μg/mL to about 50 μg/mL, about 0.5 μg/mL to about 10, 20, 30, 40 or 50 μg/mL, about 1 μg/mL to about 10 μg/mL, or about 2 μg/mL, in the composition.

### MiHA-Specific TCRs and T Lymphocytes

As noted herein, cells such as APCs that express a MHC class I molecule (e.g., HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 and/or HLA-B57 molecule) loaded with or bound to any one of, or any combination of, the MiHA peptides defined herein, may be used for stimulating/amplifying CD8⁺ T lymphocytes in vivo or ex vivo. Accordingly, in another aspect, the present disclosure provides T cell receptor (TCR) molecules capable of interacting with or binding the herein-mentioned MHC class I molecule/MiHA peptide complex, and nucleic acid molecules encoding such TCR molecules, and vectors comprising such nucleic acid molecules. A TCR according to the present disclosure is capable of specifically interacting with or binding a MiHA peptide loaded on, or presented by, a MHC class I molecule (e.g., HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule), preferably at the surface of a living cell in vitro or in vivo. A TCR and in particular nucleic acids encoding a TCR of the disclosure may for instance be applied to genetically transform/modify T lymphocytes (e.g., CD8⁺ T lymphocytes) or other types of lymphocytes generating new T lymphocyte clones that specifically recognize a MHC class I MiHA peptide complex. In a particular embodiment, T lymphocytes (e.g., CD8⁺ T lymphocytes) obtained from a patient are transformed to express one or more TCRs that recognize MiHA peptide and the transformed cells are administered to the patient (autologous cell transfusion). In a particular embodiment, T lymphocytes (e.g., CD8⁺ T lymphocytes) obtained from a donor are transformed to express one or more TCRs that recognize MiHA peptide and the transformed cells are administered to a recipient (allogenic cell transfusion). In another embodiment, the disclosure provides a T lymphocyte e.g., a CD8⁺ T lymphocyte transformed/transfected by a vector or plasmid encoding a MiHA peptide-specific TCR. In a further embodiment the disclosure provides a method of treating a patient with autologous or allogenic cells transformed with a MiHA-specific TCR. In yet a further embodiment the use of a MiHA specific TCR in the manufacture of autologous or allogenic cells for treating of cancer is provided.

In some embodiments patients treated with the compositions (e.g., pharmaceutical compositions) of the disclosure are treated prior to or following treatment with allogenic stem cell transplant (ASCL), allogenic lymphocyte infusion or autologous lymphocyte infusion. Compositions of the disclosure include: allogenic T lymphocytes (e.g., CD8⁺ T lymphocyte) activated ex vivo against a MiHA peptide; allogenic or autologous APC vaccines loaded with a MiHA peptide; MiHA peptide vaccines and allogenic or autologous T lymphocytes (e.g., CD8⁺ T lymphocyte) or lymphocytes transformed with a MiHA-specific TCR. The method to provide T lymphocyte clones capable of recognizing an MiHA peptide according to the disclosure may be generated for and can be specifically targeted to tumor cells expressing the MiHA in a subject (e.g., graft recipient), for example an ASCT and/or donor lymphocyte infusion (DLI) recipient. Hence the disclosure provides a CD8⁺ T lymphocyte encoding and expressing a T cell receptor capable of specifically recognizing or binding a MiHA peptide/MHC class I molecule complex. Said T lymphocyte (e.g., CD8⁺ T lymphocyte) may be a recombinant (engineered) or a naturally selected T lymphocyte. This specification thus provides at least two methods for producing CD8⁺ T lymphocytes of the disclosure, comprising the step of bringing undifferentiated lymphocytes into contact with a MiHA peptide/MHC class I molecule complex (typically expressed at the surface of cells, such as APCs) under conditions conducive of triggering T cell activation and expansion, which may be done in vitro or in vivo (i.e. in a patient administered with a APC vaccine wherein the APC is loaded with a MiHA peptide or in a patient treated with a MiHA peptide vaccine). Using a combination or pool of MiHA peptides bound to MHC class I molecules, it is possible to generate a population CD8⁺ T lymphocytes capable of recognizing a plurality of MiHA peptides. Alternatively, MiHA-specific or targeted T lymphocytes may be produced/generated in vitro or ex vivo by cloning one or more nucleic acids (genes) encoding a TCR (more specifically the alpha and beta chains) that specifically binds to a MHC class I molecule/MiHA complex (i.e. engineered or recombinant CD8⁺ T lymphocytes). Nucleic acids encoding a MiHA-specific TCR of the disclosure, may be obtained using methods known in the art from a T lymphocyte activated against a MiHA peptide ex vivo (e.g., with an APC loaded with a MiHA peptide); or from an individual exhibiting an immune response against peptide/MHC molecule complex. MiHA-specific TCRs of the disclosure may be recombinantly expressed in a host cell and/or a host lymphocyte obtained from a graft recipient or graft donor, and optionally differentiated in vitro to provide cytotoxic T lymphocytes (CTLs). The nucleic acid(s) (transgene(s)) encoding the TCR alpha and beta chains may be introduced into a T cells (e.g., from a subject to be treated or another individual) using any suitable methods such as transfection (e.g., electroporation) or transduction (e.g., using viral vector). The engineered CD8⁺ T lymphocytes expressing a TCR specific for a MiHA may be expanded in vitro using well known culturing methods.

The present disclosure provides isolated CD8⁺ T lymphocytes that are specifically induced, activated and/or amplified (expanded) by a MiHA peptide (i.e., a MiHA peptide bound to MHC class I molecules expressed at the surface of cell), or a combination of MiHA peptides. The present disclosure also provides a composition comprising CD8⁺ T lymphocytes capable of recognizing an MiHA peptide, or a combination thereof, according to the disclosure (i.e., one or more MiHA peptides bound to MHC class I molecules) and said MiHA peptide(s). In another aspect, the present disclosure provides a cell population or cell culture (e.g., a CD8⁺ T lymphocyte population) enriched in CD8⁺ T lymphocytes that specifically recognize one or more MHC class I molecule/MiHA peptide complex(es) as described herein. Such enriched population may be obtained by performing an ex vivo expansion of specific T lymphocytes using cells such as APCs that express MHC class I molecules loaded with (e.g. presenting) one or more of the MiHA peptides disclosed herein. “Enriched” as used herein means that the proportion of MiHA-specific CD8⁺ T lymphocytes in the population is significantly higher relative to a native population of cells, i.e. which has not been subjected to a step of ex vivo-expansion of specific T lymphocytes. In a further embodiment, the proportion of MiHA-specific CD8⁺ T lymphocytes in the cell population is at least about 0.5%, for example at least about 1%, 1.5%, 2% or 3%. In some embodiments, the proportion of MiHA-specific CD8⁺ T lymphocytes in the cell population is about 0.5 to about 10%, about 0.5 to about 8%, about 0.5 to about 5%, about 0.5 to about 4%, about 0.5 to about 3%, about 1% to about 5%, about 1% to about 4%, about 1% to about 3%, about 2% to about 5%, about 2% to about 4%, about 2% to about 3%, about 3% to about 5% or about 3% to about 4%. Such cell population or culture (e.g., a CD8⁺ T lymphocyte population) enriched in CD8⁺ T lymphocytes that specifically recognizes one or more MHC class I molecule/peptide (MiHA) complex(es) of interest may be used in MiHA-based cancer immunotherapy, as detailed below. In some embodiments, the population of MiHA-specific CD8⁺ T lymphocytes is further enriched, for example using affinity-based systems such as multimers of MHC class I molecule loaded (covalently or not) with the MiHA peptide(s) defined herein. Thus, the present disclosure provides a purified or isolated population of MiHA-specific CD8⁺ T lymphocytes, e.g., in which the proportion of MiHA-specific CD8⁺ T lymphocytes is at least about 50%, 60%, 70%, 80%, 85%, 90%, 95%, 96%, 97%, 98%, 99% or 100%.

### MiHA-Based Cancer Immunotherapy

The present disclosure further relates to the use of any peptide, nucleic acid, expression vector, T cell receptor, cell (e.g., T lymphocyte, APC), and/or composition according to the present disclosure, or any combination thereof, as a medicament or in the manufacture of a medicament. In an embodiment, the medicament is for the treatment of cancer, e.g., cancer vaccine. The present disclosure relates to any peptide, nucleic acid, expression vector, T cell receptor, cell (e.g., T lymphocyte, APC), and/or composition (e.g., vaccine composition) according to the present disclosure, or any combination thereof, for use in the treatment of cancer e.g., as a cancer vaccine. The MiHA peptide sequences identified herein may be used for the production of synthetic peptides to be used i) for in vitro priming and expansion of MiHA-specific T cells to be injected into transplant (AHCT) recipients and/or ii) as vaccines to boost the graft-vs.-tumor effect (GvTE) in recipients of MiHA-specific T cells, subsequent to the transplantation. The potential impact of the therapeutic methods provided by the present disclosure, MiHA-targeted cancer immunotherapy is significant. For hematologic cancers (e.g., leukemia, lymphoma and myeloma), the use of anti-MiHA T cells may replace conventional AHCT by providing superior anti-tumor activity without causing GvHD. It may benefit many patients with hematologic malignancy who cannot be treated by conventional AHCT because their risk/reward (GvHD/GVT) ratio is too high. Finally, since studies in mice have shown that MiHA-targeted immunotherapy may be effective for treatment of solid tumors, MiHA-based cancer immunotherapy may be used for MiHA-targeted therapy of non-hematologic cancers, such as solid cancers. In one embodiment, the cancer is leukemia including but not limited to acute lymphoblastic leukemia (ALL), acute myeloid leukemia (AML), chronic lymphocytic leukemia (CLL) chronic myeloid leukemia (CML), Hairy cell leukemia (HCL), T-cell prolymphocytic leukemia (T-PLL), Large granular lymphocytic leukemia or Adult T-cell leukemia. In another embodiment, the cancer is lymphoma including but not limited to Hodgkin lymphoma (HL), non-Hodgkin lymphoma (NHL), Burkitt's lymphoma, Precursor T-cell leukemia/lymphoma, Follicular lymphoma, Diffuse large B cell lymphoma, Mantle cell lymphoma, B-cell chronic lymphocytic leukemia/lymphoma or MALT lymphoma. In a further embodiment, the cancer is a myeloma (multiple myeloma) including but not limited to plasma cell myeloma, myelomatosis, and Kahler's disease.

In another aspect, the present disclosure provides the use of a MiHA peptide described herein, or a combination thereof (e.g. a peptide pool), as a vaccine for treating cancer in a subject. The present disclosure also provides the MiHA peptide described herein, or a combination thereof (e.g. a peptide pool), for use as a vaccine for treating cancer in a subject. In an embodiment, the subject is a recipient of MiHA-specific CD8⁺ T lymphocytes. Accordingly, in another aspect, the present disclosure provides a method of treating cancer (e.g., of reducing the number of tumor cells, killing tumor cells), said method comprising administering (infusing) to a subject in need thereof an effective amount of CD8⁺ T lymphocytes recognizing (i.e. expressing a TCR that binds) one or more MHC class I molecule/MiHA peptide complexes (expressed at the surface of a cell such as an APC). In an embodiment, the method further comprises administering an effective amount of the MiHA peptide, or a combination thereof, and/or a cell (e.g., an APC such as a dendritic cell) expressing MHC class I molecule(s) loaded with the MiHA peptide(s), to said subject after administration/infusion of said CD8⁺ T lymphocytes. In yet a further embodiment, the method comprises administering to a subject in need thereof a therapeutically effective amount of a dendritic cell loaded with one or more MiHA peptides. In yet a further embodiment the method comprises administering to a patient in need thereof a therapeutically effective amount of an allogenic or autologous cell that expresses a recombinant TCR that binds to a MiHA peptide presented by a MHC class I molecule.

In another aspect, the present disclosure provides the use of CD8⁺ T lymphocytes that recognize one or more MHC class I molecules loaded with (presenting) a MiHA peptide, or a combination thereof, for treating cancer (e.g., of reducing the number of tumor cells, killing tumor cells) in a subject. In another aspect, the present disclosure provides the use of CD8⁺ T lymphocytes that recognize one or more MHC class I molecules loaded with (presenting) a MiHA peptide, or a combination thereof, for the preparation/manufacture of a medicament for treating cancer (e.g., fir reducing the number of tumor cells, killing tumor cells) in a subject. In another aspect, the present disclosure provides CD8⁺ T lymphocytes (cytotoxic T lymphocytes) that recognize one or more MHC class I molecule(s) loaded with (presenting) a MiHA peptide, or a combination thereof, for use in the treatment of cancer (e.g., for reducing the number of tumor cells, killing tumor cells) in a subject. In a further embodiment, the use further comprises the use of an effective amount of a MiHA peptide (or a combination thereof), and/or of a cell (e.g., an APC) that expresses one or more MHC class I molecule(s) loaded with (presenting) a MiHA peptide of formula I, after the use of said MiHA-specific CD8⁺ T lymphocytes. In an embodiment, the subject infused or treated with MiHA-specific CD8 T-cells has received prior treatment with AHCT or donor lymphocyte infusions (i.e. lymphocytes, including T-cells, that have not been activated in vitro against a MiHA peptide presented by a MHC class I molecule).

The present disclosure also provides a method of generating an immune response against tumor cells expressing human class I MHC molecules loaded with any of the MiHA peptide disclosed herein or combination thereof in a subject, the method comprising administering cytotoxic T lymphocytes that specifically recognizes the class I MHC molecules loaded with the MiHA peptide or combination of MiHA peptides. The present disclosure also provides the use of cytotoxic T lymphocytes that specifically recognizes class I MHC molecules loaded with any of the MiHA peptide or combination of MiHA peptides disclosed herein for generating an immune response against tumor cells expressing the human class I MHC molecules loaded with the MiHA peptide or combination thereof.

In a further embodiment, the cancer is a hematologic cancer, e.g., leukemia, lymphoma and myeloma. In an embodiment, the cancer is leukemia.

### Treatment and Donor Selection Methods

Allogenic therapeutic cells described herein express a TCR that recognizes a MiHA peptide that is presented by a patient's (recipient's) tumor cells but not presented by cells of the donor. The disclosure provides a method of selecting an effective therapeutic composition for a patient having a cancer (e.g., a hematological cancer) comprising: (a) obtaining a biological sample from the patient; (b) determining the presence or absence of one or more SNPs selected from Table II, VI or VII; (c) determining the expression of RNA or protein products corresponding to one or more of the SNPs provided in Table II, VI or VII in a tumor sample from the patient. For treatment with allogenic cells: (a) a donor that does not express a genetic variant, corresponding to a MiHA peptide (i.e. those provided in Table II, VI or VII herein) presented by MHC class I molecules expressed by the recipient's cancer cells is selected (b) lymphocytes are obtained from the donor and (c) CD8⁺ T lymphocytes specific for the presented MiHA peptide are prepared using the methods provided herein and administered to the patient. Alternatively, allogenic cells obtained from the selected donor, one that does not express the MiHA peptide of interest, can be genetically transformed to express a TCR against the MiHA of interest and administered to the patient.

For treatment with autologous cells, autologous T lymphocytes expressing a TCR that recognizes one or more MiHA peptide(s) presented by MHC class I molecules present on the cell surface of a patient's cancer cells is administered. The disclosure provides a method of selecting a T lymphocyte therapy for a patient comprising: (a) obtaining a biological sample from the patient; (b) determining the presence or absence of one or more SNPs selected from Table II, VI or VII; (c) determining the expression of RNA or protein products corresponding to one or more of the SNPs provided in Table II, VI or VII in a tumor sample from the patient.

To determine which variant of a given MiHA that should be used in the treatment of a subject (e.g., using MiHA No. 2 (A/SEIEQKIKEY) as an example, to determine which of AEIEQKIKEY or SEIEQKIKEY should be used), the allelic variant expressed by the subject should be first determined. The amino acid substitutions in the proteins as well as the nucleotide substitutions in the transcripts corresponding to the MiHAs described herein (Table II, VI or VII) may be easily identified by the skilled person, for example using the information provided in public databases. For example, Tables II, VI and VII include the reference/identification No. for MiHAs in the dbSNP database, which provides detailed information concerning the variations at the genomic, transcript and protein levels. Based on this information, the determination of the variant (polymorphism or single nucleotide polymorphism (SNP)) expressed by the subject may be readily performed at the nucleic acid and/or protein level on a sample by a number of methods which are known in the art. Table II also includes the reference ID in the Ensembl database for the genes from which the MiHA peptides are derived.

Examples of suitable methods for detecting alterations at the nucleic acid level include sequencing the relevant portion (comprising the variation) of the nucleic acid of interest (e.g., a mRNA, cDNA or genomic DNA encoding the MiHAs), hybridization of a nucleic acid probe capable of specifically hybridizing to a nucleic acid of interest comprising the polymorphism (the first allele) and not to (or to a lesser extent to) a corresponding nucleic acid that do not comprise the polymorphism (the second allele) (under comparable hybridization conditions, such as stringent hybridization conditions), or vice-versa; restriction fragment length polymorphism analysis (RFLP); Amplified fragment length polymorphism PCR (AFLP-PCR); amplification of a nucleic acid fragment using a primer specific for one of the allele, wherein the primer produces an amplified product if the allele is present and does not produce the same amplified product when the other allele is used as a template for amplification (e.g., allele-specific PCR). Other methods include in situ hybridization analyses and single-stranded conformational polymorphism analyses. Further, nucleic acids of interest may be amplified using known methods (e.g., polymerase chain reaction [PCR]) prior to or in conjunction with the detection methods noted herein. The design of various primers for such amplification is known in the art. The nucleic acid (mRNA) may also be reverse transcribed into cDNA prior to analysis.

Examples of suitable methods for detecting alterations/polymorphisms at the polypeptide level include sequencing of the relevant portion (comprising the variation) of the polypeptide of interest, digestion of the polypeptide followed by mass spectrometry or HPLC analysis of the peptide fragments, wherein the variation/polymorphism of the polypeptide of interest results in an altered mass spectrometry or HPLC spectrum; and immunodetection using an immunological reagent (e.g., an antibody, a ligand) which exhibits altered immunoreactivity with a polypeptide comprising the alteration (first allele) relative to a corresponding native polypeptide not comprising the alteration (second allele), for example by targeting an epitope comprising the amino acid variation. Immunodetection can measure the amount of binding between a polypeptide molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots, and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

In one embodiment the disclosure provides a method of selecting an effective therapeutic composition for a patient comprising: (a) isolating MHC class I presented peptides from cancer cells (e.g., hematologic cancer cells) from the patient; and (b) identifying the presence or absence of one or more MiHA peptides depicted in Table I among said MHC class I presented peptides. In a further embodiment, the identification of the presence or absence of the one or more MiHA peptides depicted in Table I is performed by mass spectrometry and/or using an antibody detection reagent that is selective for the one or more MiHA peptides. Detecting or identifying MiHA peptides using mass spectrometry can be performed using methods known in the art such as those described in PCT publications Nos. WO2014/026277 and WO/2016/127249. Mass spectrometry (MS) sequencing of MiHA peptides presented by MHC class I molecules, which have been isolated from a sample of cancer cells, involves comparing an MS spectrum obtained for an isolated and digested peptide to spectra computed in silico for a MiHA peptide. Therapeutic allogenic T lymphocytes described herein, for treating a patient with cancer, target MHC class I molecules presenting one or more MiHA peptides that is/are expressed by cancer cells in the patient but not expressed by the donor's cells. As such, selecting an appropriate donor for generating allogenic T lymphocytes of the disclosure involves genotyping candidate donors for the presence or absence of one or more single nucleotide polymorphisms provided in Table II, VI or VII.

In one embodiment, the disclosure provides a method of selecting an effective immunotherapy treatment (i.e. MHC class I molecule/MiHA peptide complex target) for a patient with cancer comprising: determining the presence of MiHA peptides presented by MHC class I molecules in tumor cells from the patient. In another embodiment the disclosure provides a method of screening candidate allogenic cell donors for a patient comprising determining the presence or absence of one or more SNPs selected from those provided in Table II in a biological sample from the donor. In an embodiment, the presence or absence of a SNP corresponding to a MiHA peptide known to be presented by MHC class I molecule in cancer cells obtained from a patient is determined in candidate donors. In a further embodiment, biological samples obtained from candidate allogenic donors are genotyped to determine the presence or absence of one or more SNPs known to be carried by a patient, wherein the SNPs detected are selected from those provided in Table II. In a further embodiment the disclosure provides a genotyping system comprising a plurality of oligonucleotide probes conjugated to a solid surface for detection of a plurality of SNPs selected from Table II, VI or VII.

For example, to determine which variant of MiHA No. 2 (AEIEQKIKEY or SEIEQKIKEY) should be used in the treatment of a subject, it should be determined on a sample from the subject using any suitable method (sequencing, etc.) whether (i) a transcript from the RASSF1 gene comprises a G or T at a position corresponding to position 528 of Ensembl Transcript ID No. ENST00000359365.8 (ENSG00000068028); (ii) the nucleotide corresponding to position 50322115 of chromosome 3 in human genome assembly GRCh38.p7 is G or T; and/or (iii) a RASSF1 polypeptide comprises an alanine or serine residue at a position corresponding to position 133 of the polypeptide encoded by Ensembl Transcript ID No. ENST00000359365.8. If (i) the transcript from the RASSF1 gene comprises a G at a position corresponding to position 528 of Ensembl Transcript ID No. ENST00000359365.8; (ii) the nucleotide corresponding to position 50322115 of chromosome 3 in human genome assembly GRCh38.p7 is G; and/or (iii) the RASSF1 polypeptide comprises an alanine residue at a position corresponding to position 133 of the polypeptide encoded by Ensembl Transcript ID No. ENST00000359365.8, MiHA variant AEIEQKIKEY should be used. Alternatively, if (i) the transcript from the RASSF1 gene comprises a T at a position corresponding to position 528 of Ensembl Transcript ID No. ENST00000359365.8; (ii) the nucleotide corresponding to position 50322115 of chromosome 3 in human genome assembly GRCh38.p7 is T; and/or (iii) the RASSF1 polypeptide comprises a serine residue at a position corresponding to position 133 of the polypeptide encoded by Ensembl Transcript ID No. ENST00000359365.8, MiHA variant SEIEQKIKEY should be used. The same approach may be applied to determine which variant of any of MiHAs Nos. 1 and 3-138 of Table I should be used in a given subject. MiHAs No. 4 may only be used in male subjects (since the encoding gene is located on chromosome Y, the MiHA is only expressed in male subjects).

In an embodiment, the herein-mentioned CD8⁺ T lymphocytes are in vitro or ex vivo expanded CD8⁺ T lymphocytes, as described herein. Expanded CD8⁺ T lymphocytes may be obtained by culturing primary CD8⁺ T lymphocytes (from an allogenic donor) under conditions permitting the proliferation (amplification) and/or differentiation of the CD8⁺ T lymphocytes. Such conditions typically include contacting the CD8⁺ T lymphocytes with cells, such as APCs, expressing at their surface the MiHA peptide(s)/MHC complexes of interest, in the presence of a suitable medium (medium for hematopoietic/lymphoid cells, e.g., X-VIVO™15 and AIM-V®) growth factors and/or cytokines such as IL-2, IL-7 and/or IL-15 (see, e.g., Montes et al., Clin Exp Immunol. 2005 November; 142(2):292-302). Such expanded CD8⁺ T lymphocytes are then administered to the recipient, for example through intravenous infusion. Methods and conditions for amplifying and preparing antigen-specific CD8⁺ T lymphocytes for adoptive immunotherapy are disclosed, for example, in DiGiusto et al., Cytotherapy 2007; 9(7): 613-629 and Bollard et al., Cytotherapy. 2011 May; 13(5): 518-522). Standard Operating procedures (SOPs) for amplifying antigen-specific CD8⁺ T lymphocytes are available from the Center for Cell and Gene Therapy, Baylor College of Medicine, Texas Children's Hospital, The Methodist Hospital, Houston, Tex., USA (see Sili et al., Cytotherapy. 2012 January; 14(1): 7-11, Supplementary Material). In an embodiment, the subject (recipient) is an allogeneic stem cell transplantation (ASCT) or donor lymphocyte infusion (DLI) recipient.

In another aspect, the present disclosure provides a method of culturing or expanding CD8⁺ T lymphocytes (e.g., for adoptive T-cell immunotherapy), said method comprising (a) culturing CD8⁺ T lymphocytes from a first individual not expressing a variant of a MiHA peptide in the presence of cells expressing a MHC class I molecule of a suitable HLA allele (e.g., HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule) loaded with said variant of the MiHA peptide, under conditions suitable for CD8⁺ T lymphocyte expansion/proliferation. In another aspect, the disclosure provides a method of producing/manufacturing cells for cellular immunotherapy comprising: culturing human lymphocytes in the presence of APC comprising a MiHA peptide presented by a MHC class I molecule, wherein the MHC class I molecule is of the HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 subtype. The human T lymphocyte used in this method is an allogenic cell i.e. a cell obtained from a donor being manufactured for treating a recipient with an allogenic cell. In another aspect, the disclosure provides a method of producing/manufacturing cells for cellular immunotherapy comprising: (a) obtaining lymphocytes (e.g., T lymphocytes) from a cultured cell line and (b) culturing the cells in the presence of APC comprising a MHC class I molecule/MiHA peptide complex wherein the MHC class I molecule is of the HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 subtype. The human T lymphocyte used in the method is preferably an allogenic cell, i.e. a cell obtained from a donor being manufacture for treating a recipient with an allogenic cell. In a further embodiment, the disclosure provides a method of producing/manufacturing cells for cellular immunotherapy comprising: (a) obtaining cells from a donor, e.g., a patient having a hematopoietic cancer (e.g., leukemia) or a healthy individual, for example by leukapheresis, and (b) transforming the cells with a recombinant TCR that binds to a MHC class I molecule/MiHA peptide complex. In a further embodiment, the disclosure provides a method of manufacturing cells for cellular immunotherapy comprising transforming a human cell line with a recombinant TCR that binds with to a MHC class I molecule/MiHA peptide complex as defined herein.

In another aspect, the present disclosure provides a method of expanding CD8⁺ T lymphocytes for adoptive T-cell immunotherapy, said method comprising (a) determining which variant of any of MiHA Nos. 1-138 or 1-81, preferably MiHA Nos. 3, 5, 8-15, 25-28, 30-33, 36-49, 54-61, 65-66, 68-77 and 79-81 is expressed by a subject (recipient), culturing CD8⁺ T lymphocytes from a candidate donor in the presence of cells expressing a MHC class I molecule of a suitable HLA allele (e.g., HLA-A1, HLA-A3, HLA-A11, HLA-A24, HLA-A29, HLA-A32, HLA-B7, HLA-B8, HLA-B13, HLA-B14, HLA-B15, HLA-B18, HLA-B27, HLA-B35, HLA-B40, HLA-B44 or HLA-B57 molecule) loaded with the MiHA variant expressed by the subject, under conditions suitable for CD8⁺ T lymphocyte expansion, wherein said candidate donor does not express the MiHA variant (expressed by the subject (recipient)). In another aspect, the disclosure provides a method of selecting a therapeutic approach for a patient having cancer, for example leukemia: (a) detecting the presence of a MiHA peptide presented by a MHC class I molecule expressed in cancer (e.g., leukemic) cells obtained from the patient; and (b) determining the presence or absence of a SNP corresponding to the MiHA peptide detected in step (a), as indicated in Table II, in biological samples obtained from candidate donors.

In another aspect, the disclosure provides a method of preparing a therapeutic composition for a patient having leukemia: (a) detecting the presence of a MiHA peptide presented by a MHC class I molecule expressed in leukemic cells obtained from the patient; (b) obtaining lymphocytes from the patient by leukapheresis, and (c) transforming said lymphocytes with a TCR that recognizes the presented MiHA peptide detected in step (a). In another aspect, the disclosure provides a method of preparing a therapeutic composition for a patient having, for example leukemia: (a) genotyping the patient to determine the presence of a plurality of SNPs selected from Table II, VI or VII; (b) determining the presence of one of the SNPs in the patient (c) obtaining cells from the patient by leukapheresis, and (d) incubating said cells with a APC expressing a MHC class I molecule/MiHA peptide complex, comprising a MiHA peptide that contains the polymorphism encoded by the SNP present in said patient.

Again using MiHA No. 2 as a representative example to illustrate the method, if it is determined that in a sample from the subject: (i) the transcript from the RASSF1 gene comprises a G at a position corresponding to position 528 of Ensembl Transcript ID No. ENST00000359365.8; (ii) the nucleotide corresponding to position 50322115 of chromosome 3 in human genome assembly GRCh38.p7 is G; and/or (iii) the RASSF1 polypeptide comprises an alanine residue at a position corresponding to position 133 of the polypeptide encoded by Ensembl Transcript ID No. ENST00000359365.8, the CD8⁺ T lymphocytes from the candidate donor are cultured in the presence of cells expressing a MHC class I molecule of the HLA-B18, HLA-B40 and/or HLA-B44 alleles loaded with MiHA variant AEIEQKIKEY under conditions suitable for CD8⁺ T lymphocyte expansion. Alternatively, if it is determined that in a sample from the subject: (i) the transcript from the RASSF1 gene comprises a T at a position corresponding to position 528 of Ensembl Transcript ID No. ENST00000359365.8; (ii) the nucleotide corresponding to position 50322115 of chromosome 3 in human genome assembly GRCh38.p7 is T; and/or (iii) the RASSF1 polypeptide comprises a serine residue at a position corresponding to position 133 of the polypeptide encoded by Ensembl Transcript ID No. ENST00000359365.8, the CD8⁺ T lymphocytes from the candidate donor are cultured in the presence of cells expressing a MHC class I molecule of the HLA-B18, HLA-B40 and/or HLA-B44 alleles loaded with MiHA variant SEIEQKIKEY under conditions suitable for CD8⁺ T lymphocyte expansion. The same approach may be applied to any of MiHAs Nos. 1 and 3-138 defined herein.

In an embodiment, the present disclosure provides a method of treating cancer, said method comprising (i) expanding CD8⁺ T lymphocytes recognizing a MHC class I molecule loaded with a peptide of formula I for adoptive T-cell immunotherapy according to the method defined herein; and (ii) administering (infusing) to a subject in need thereof an effective amount of the expanded CD8⁺ T lymphocytes. In one embodiment, the method further comprises administering an effective amount of the peptide of formula I, and/or a cell (e.g., an APC) expressing MHC class I molecule loaded with a MiHA peptide of formula I, to said subject after administration/infusion of said CD8⁺ T lymphocytes. In an embodiment, the herein-mentioned cancer comprises tumor cells expressing the genes encoding MiHAs Nos. Nos. 1-138 or 1-81, preferably MiHA Nos. 3, 5, 8-15, 25-28, 30-33, 36-49, 54-61, 65-66, 68-77 and/or 79-81 set forth in Table I, or a combination thereof.

### MODE(S) FOR CARRYING OUT THE INVENTION

The present invention is illustrated in further details by the following non-limiting examples.

### Example 1: Materials and Methods (for Examples 2 and 3)

The MiHAs were identified according to the method/strategy described in PCT publications Nos. WO 2014/026277 and WO 2016/127249.

Cell Culture.

Peripheral blood mononuclear cells (PBMCs) were isolated from blood samples of 9 female and 9 male healthy volunteers expressing at least one of the following common alleles HLA-A*01:01, HLA-A*03:01, HLA-A*11:01, HLA-A*24:02, HLA-A*29:02, HLA-A*32:01, HLA-B*07:02, HLA-B*08:01, HLA-B*13:02, HLA-B*14:02, HLA-B*15:01, HLA-B*18:01, HLA-B*27:05, HLA-B*35:01, HLA-B*40:01, HLA-B*44:02 and HLA-B*57:01. Epstein-Barr virus (EBV)-transformed B lymphoblastoid cell lines (B-LCLs) were derived from PBMCs with Ficoll-Paque™ Plus (Amersham) as previously described (Tosato and Cohen, 2007). Established B-LCLs were maintained in RPMI 1640 medium supplemented with 10% fetal bovine serum, 25 mM of HEPES, 2 mM L-glutamine and penicillin-streptomycin (all from Invitrogen®).

DNA Extraction.

Genomic DNA was extracted from 5 million B-LCLs using the PureLink™ Genomic DNA Mini Kit (Invitrogen®) according to the manufacturer's instructions. DNA was quantified and quality-assessed using the Taqman® RNase P Detection Reagents Kit (Life Technologies®).

HLA Typing.

High-resolution HLA genotyping was performed using 500 ng of genomic DNA at the Maisonneuve-Rosemont Hospital.

Preparation of Genomic DNA Libraries.

Genomic libraries were constructed from 200 ng of genomic DNA using the Ion AmpliSeq™ Exome RDY Library Preparation Kit (Life Technologies®) following the manufacturer's protocol. This included the following steps: amplification of targets, partial digestion of primer sequences, ligation of Ion Xpress™ barcode adapters to the amplicons, purification of the library using AMPure® XP reagent (Beckman Coulter®) and quantification of the unamplified library by qPCR. Library templates were then prepared and loaded onto Ion Proton™ I chips using the Ion PI™ IC 200 kit and the Ion Chef™ System.

Exome Sequencing and Variant Calling.

Two exome libraries were sequenced per chip on an Ion Proton™ Sequencer using the default parameters of AmpliSeq™ exome libraries. Variant calling was done using the Torrent Variant Caller plugin with the “germ Line Proton—Low Stringency” parameter of the Ion reporter Software.

RNA Extraction.

Total RNA was isolated from 5 million B-LCLs using TRizol RNA reagent (Life Technologies®) including DNase I treatment (Qiagen®) according to the manufacturer's instructions. Total RNA was quantified using the NanoDrop™ 2000 (Thermo Scientific®) and RNA quality was assessed with the 2100 Bioanalyzer™ (Agilent Technologies®).

Preparation of Transcriptome Libraries.

Libraries were generated from 1 μg of total RNA using the TruSeq™ RNA Sample Prep Kit (v2) (RS-930-1021, Illumina®) following the manufacturer's protocol. Briefly, poly-A mRNA was purified using poly-T oligo-attached magnetic beads using two rounds of purification. During the second elution of the poly-A RNA, the RNA was fragmented and primed for cDNA synthesis. Reverse transcription (RT) of the first strand was done using random primers and SuperScript™ II (InvitroGene®). A second round of RT was also done to generate a double-stranded cDNA, which was then purified using Agencourt AMpure™ XP PCR purification system (Beckman Coulter®). End repair of fragmented cDNA, adenylation of the 3′ ends and ligation of adaptors were done following the manufacturer's protocol. Enrichment of DNA fragments containing adapter molecules on both ends was done using 15 cycles of PCR amplification and the Illumina® PCR mix and primers cocktail.

Whole Transcriptome Sequencing (RNA-Seq).

Paired-end (2×100 bp) sequencing was performed using the Illumina HiSeg2000™ machine running TruSeq™ v3 chemistry. Cluster density was targeted at around 600-800k clusters/mm². Two transcriptomes were sequenced per lane (8 lanes per slide). Details of the Illumina sequencing technologies can be found at https://www.illumina.com/techniques/sequencing.html.

Read Mapping.

Sequence data were mapped to the human reference genome (hg19, UCSC) using the Ilumina Casava™ 1.8.1 and the Eland™ v2 mapping softwares. First, the *.bcl files were converted into compressed FASTQ files, following by demultiplexing of separate multiplexed sequence runs by index. Then, single reads were aligned to the human reference genome including the mitochondrial genome using the multiseed and gapped alignment method. Reads that mapped at 2 or more locations (multireads) were not included in further analyses. An additional alignment was done against splice junctions and contaminants (ribosomal RNA).

Identification of Single Nucleotide Variations in the Transcriptome.

First, the list of all single nucleotide variations observed between the reference genome (GRCh37.p2, NCBI) and the sequenced transcriptome of each of the individuals was retrieved. This was done using the SNP calling program Casava™ v1.8.2 from Ilumina® (https://support.illumina.com/sequencing/sequencing_software/casava.html). Only high confidence single nucleotide variations (Qmax_gt value>20) and that were observed in at least 3 reads (coverage 3) were considered. SNVs with Qmax_gt value below this threshold were assigned with the reference base instead. This strategy was used to identify single nucleotide variations at the transcript level between each of the individuals and the reference genome.

In Silico Translated Transcriptome.

The sequences containing the identified single nucleotide variations of each individual were further processed. For each sequence, all transcripts reported in Ensembl (http://useast.ensembl.org/info/data/ftp/index.html, Flicek et al., Ensembl 2012, Nucleic Acids Research 2012 40 Database issue:D84-D90) were retrieved and in silico translated into proteins using an in-house software pyGeno version (python package pyGeno 1.1.7, https://pypi.python.org/pypi/pyGeno/1.1.7), Granados et al., 2012 (PMID: 22438248)). The in silico translated transcriptomes included cases in which more than one non-synonymous polymorphism was found for a given position. Considering that most MAPs have a maximum length of 11 amino acids (33 bp), the existence of a heterozygous position could lead to MiHA variants in a window of 21 (66 bp) amino acids centered at each ns-SNP. When a window contained more than one ns-SNP, all possible combinations were translated. The number of combinations affected by one ns-SNP was limited to 10,240 to limit the size of the file. In this way, a list of all possible sequences of at most 11 amino acids affected by ns-SNPs was obtained and included in the individual-specific protein databases, which were further used for the identification of MAPs.

Mass Spectrometry and Peptide Sequencing.

3 to 4 biological replicates of 5-6×10⁸ exponentially growing B-LCLs were prepared from each individual. MHC class I-associated peptides were released by mild acid treatment, pretreated by desalting with an HLB cartridge and filtered with a 3,000 Da cut-off column as previously described (Caron et al. 2011 (PMID: 21952136)). Samples were further processed according to two different methods. In the first method, samples were vacuum dried, resuspended in SCX Reconstitution Solution (Protea®) and separated into six fractions using SCX spintips (Protea®) and an ammonium formate buffer step gradient (50, 75, 100, 300, 600, 1500 mM). Vacuum dried fractions were resuspended in 5% acetonitrile, 0.2% formic acid and analyzed by LC-MS/MS using an Eksigent® LC system coupled to a LTQ-Orbitrap ELITE™ mass spectrometer (Thermo Electron®). Peptides were separated on a custom C18 reversed phase column (pre-column: 0.3 mm i.d.×5 mm, analytical column: 150 μm i.d.×100 mm; Jupiter® C18 3 μm 300 Å) using a flow rate of 600 nL/min and a linear gradient of 5-40% aqueous ACN (0.2% formic acid) in 56 min. Full mass spectra were acquired with the Orbitrap® analyzer operated at a resolving power of 60,000 (at m/z 400). Mass calibration used an internal lock mass (protonated (Si(CH₃)₂O))₆; m/z 445.120029) and mass accuracy of peptide measurements was within 5 ppm. MS/MS spectra were acquired at higher energy collisional dissociation with normalized collision energy of 28. Up to ten precursor ions were accumulated to a target value of 50,000 with a maximum injection time of 100 ms and fragment ions were transferred to the Orbitrap® analyzer operating at a resolution of 60,000 at m/z 400. In the second method, samples were split into two identical technical replicates following the 3,000 Da filtration step and vacuum-dried. One technical replicate was resuspended in 3% acetonitrile, 0.2% formic acid and analyzed by LC-MS/MS using an EASY-nLC® II system coupled to a Q-Exactive™ Plus mass spectrometer (Thermo Scientific®). Peptides were separated on a custom C18 reversed phase column as in the first method, using a flow rate of 600 nl/min and a linear gradient of 3-25% aqueous ACN (0.2% formic acid) in 146 min followed by 25-40% in 5 min. Full mass spectra were acquired with the Orbitrap® analyzer operated at a resolving power of 70,000 (at m/z 400). Mass calibration used an internal lock mass (protonated (Si(CH₃)₂O))₆; m/z 445.120029) and mass accuracy of peptide measurements was within 5 ppm. MS/MS spectra were acquired at higher energy collisional dissociation with normalized collision energy of 25. Up to twelve precursor ions were accumulated to a target value of 1,000,000 with a maximum injection time of 200 ms and fragment ions were transferred to the Orbitrap® analyser operating at a resolution of 17,500 at m/z 400.

MS/MS Sequencing and Peptide Clustering.

Database searches were performed against databases specific to each individual (see ‘in silico-generated proteome and personalized databases’ section) using PEAKS®7 (Bioinformatics Solutions Inc., http://www.bioinfor.com/). Mass tolerances for precursor and fragment ions were set to 5 p.p.m. and 0.02 Da, respectively. Searches were performed without enzyme specificity and with variable modifications for cysteinylation, phosphorylation (Ser, Thr and Tyr), oxidation (Met) and deamidation (Asn, Gln). Raw data files were converted to peptide maps comprising m/z values, charge state, retention time and intensity for all detected ions herein a threshold of 30,000 counts. Using in-house software (Proteoprofile) (Granados et al. 2014), peptide maps corresponding to all identified peptide ions were aligned together to correlate their abundances across sample replicates. PEAKS decoy-fusion approach was used to calculate the false discovery rate of quantified unique peptide sequences. The highest scored MS/MS spectra of MHC class I peptides detected in at least one of the individuals were validated manually, using Xcalibur™ software version 2.2 SP1.48 (Thermo Scientific®).

Selection of MiHAs.

Peptides were filtered by their length and those peptides with the canonical MAP length (typically 8-14 mers) were kept. The predicted binding affinity (IC₅₀) of peptides to the allelic products was obtained using NetMHC version 3.4 (http://www.cbs.dtu.dk/services/NetMHC/, Lundegaard et al., 2008 (PMID: 18413329)). Peptides with an IC₅₀ below 5,000 nM were considered as HLA binders.

MiHAs were selected according to the following criteria:

i) Presence of a reported non-synonymous SNP (nsSNP) in the peptide-coding region of the individuals leading to surface expression of the corresponding peptide(s). These constitute MiHA differences between the individuals and other individuals harboring the alternate allele for the reported SNP.
ii) Unambiguous origin of the MiHA. Hence, the MiHA has a single genetic origin in the individual's genome.
iii) The MiHA does not derive from immunoglobulins or HLA class I or class II genes since these genes are highly polymorphic and very variable between individuals.
iv) The MiHA has a reported minor allele frequency (MAF) of at least 0.05 according to the dbSNP database build 138 (NCBI) and/or the NHLBI Exome Sequencing Project (ESP).

The RNA (cDNA) and DNA sequences encoding MiHAs were manually inspected using the Integrative Genomics Viewer v2.3.25 (The Broad Institute). The UCSC Repeat Masker track was included to discard candidates that corresponded to repetitive regions.

Determination of Allele Frequency.

The minor allele frequency (MAF) of each ns-SNP was obtained from the dbSNP database build 138 (NCBI) and/or the NHLBI Exome Sequencing Project (ESP). A definition of MAF can be found here: (http://www.ncbi.nlm.nih.gov/proiects/SNP/docs/rs attributes.html. Briefly, dbSNP is reporting the minor allele frequency for each rs included in a default global population. Since this is being provided to distinguish common polymorphism from rare variants, the MAF is actually the second most frequent allele value. In other words, if there are 3 alleles, with frequencies of 0.50, 0.49, and 0.01, the MAF will be reported as 0.49. The default global population is 1000Genome phase 1 genotype data from 1094 worldwide individuals, released in the May 2011 dataset.

MS/MS Validation of MiHA Sequences.

The highest scored MS/MS spectra of all candidate MiHAs detected in at least one of the individuals were validated manually, using Xcalibur™ software version 2.2 SP1.48 (Thermo Scientific®). MS/MS spectra of the selected MiHAs were further validated using synthetic MiHA versions synthesized by Genscript. Subsequently, 250-500 fmol of each peptide were injected in the LTQ-Orbitrap ELITE™ or the Q-Exactive™ Plus mass spectrometer using the same parameters as those used to analyze the biological samples.

Determination of the Tissue Distribution of Gene Expression.

Allogeneic T cells can react against a multitude of host MiHAs expressed elsewhere than in hematopoietic/lymphoid organs and induce GVHD. Therefore, to avoid GVHD MiHA expression should not be ubiquitous. Unfortunately, current technical limitations prevent from experimentally assessing MiHA expression in these tissues by mass spectrometry. As an alternative, it was previously shown that MAPs preferentially derive from abundant transcripts (Granados et al. Blood 2012). Thus, the level of expression of transcripts coding for MiHAs could be used as an indicator of MiHAs expression. Publicly available data from Fagerberg et al., Mol Cell Proteomics 2014 13: 397-406 were used, which is part of The Human Project Atlas (THPA) (http://www.proteinatlas.org/tissue, Uhlen et al (2010). Nat Biotechnol. 28(12):1248-50), listing the expression profiles of human genes for 32 tissues. From this data, the expression level of genes coding for the identified MiHAs was obtained. Genes were then classified as “ubiquitous” if expressed in 32 tissues with a “Fragments Per Kilobase of exons per Million mapped reads (FPKM)” >10 or as “not ubiquitous” if not expressed with a FPKM >10 in all 32 tissues. Also, these data were used to calculate the ratio of MiHA genes expression in the bone marrow compared to the skin. Of note, the bone marrow samples used by from Fagerberg et al. (supra) were Ficoll™-separated preparations in which non-hematopoietic components of stroma, adipose cells, bone and vessels, as well as large portions of the fully differentiated erythropoietic and myelopoietic populations had been removed (http://www.proteinatlas.org/humanproteome/bone+marrow). Reads Per Kilobase per Million mapped reads (RPKM) values of MiHA-coding genes in AML samples were obtained from the TCGA Data Portal version 3.1.6. AML data included 179 samples of different subtypes: 16 M0, 42 M1, 41 M2, 16 M3, 36 M4, 21 M5, 2 M6, 3 M7, 2 not classified. Values were converted to Log₁₀(1,000 RPKM+1) for visualization purposes. Mean values were calculated using the 179 AMLs, expect for the Y chromosome-encoded UTY gene, for which only 95 male samples were considered.

Cumulative Number of Identified MiHAs Per Individual.

A custom software tool was used to estimate the cumulative number of HLA-A*01:01, HLA-A*02:01, HLA-A*03:01, HLA-A*11:01, HLA-A*24:02, HLA-A*29:02, HLA-A*32:01, HLA-B*07:02, HLA-B*08:01, HLA-B*13:02, HLA-B*14:02, HLA-B*15:01, HLA-B*18:01, HLA-B*27:05, HLA-B*35:01, HLA-B*40:01, HLA-B*44:02, HLA-B*44:03 or HLA-B*57:01-associated MiHAs expected for each additional individual studied. Since this number is influenced by the MiHAs present in each individual and by the order in which individuals are analyzed, the number of newly identified MiHAs expected for each additional individual studied in all combinations and permutations of groups of studied individuals was exhaustively listed. Then, the average number of MiHAs for each number of studied individuals was computed. To approximate the cumulative number of MiHAs for up to 20 individuals, a predictive curve was mapped on the data points. The curve was fitted on a function using the curve_fit tool from the “optimize” module of the “scipy” Python library (Jones E, Oliphant E, Peterson P, et al. SciPy: Open Source Scientific Tools for Python, 2001-, http://www.scipy.org/). The following equation was used to represent the cumulative number of identified MiHAs:

$$\frac{a \times x}{b + x}$$

Frequency of Therapeutic MiHA Mismatches.

In order to estimate the number of therapeutic MiHA mismatches, a bioinformatic simulation approach was used. For each ns-SNP encoding the 39 optimal MiHAs, the reported alleles were retrieved from the European-American population of the Exome Sequencing Project (ESP) or, if not available, from the European population of “The 1,000 Genomes Project” (http://www.1000genomes.org/). Next, the alleles were categorized from a peptide perspective as ‘dominant’ if the MiHA was detected by MS or known to be immunogenic, or as ‘recessive’ if the MiHA was neither detected by MS nor shown to be immunogenic. Of note, in some loci both alleles were codominant. It was assumed that the presence of a dominant allele always leads to the surface expression of the MiHA. In the case of overlapping MiHAs deriving from the same ns-SNP, the MiHA locus was considered only once. In this simulation, it was also assumed that MiHA-coding SNPs are independent events. In the case of Y chromosome-derived MiHAs (absent in females), a therapeutic mismatch occurred in all male recipient/female donor pairs. Based on the reported minor allele frequencies (MAFs), the allele frequency of the ‘dominant’ or of the ‘recessive’ MiHA was determined in all MiHA-coding loci. Assuming a female/male ratio of 1:1, 1×10⁶ unrelated donor/recipient pairs were randomly generated and virtually genotyped using increasing subsets (1 to 30) of this ranked list of MiHAs. Thus, one population was generated for each MiHA subset. The MAF of each MiHA was used as a probability to generate each individual's maternal and paternal MiHA alleles. For each MiHA subset tested, this procedure resulted in two sets of MiHA alleles (or MiHAs alleles) per individual. The number of MiHA mismatches found in each pair was determined and if at least one mismatch was achieved, a therapeutic mismatch was called. The same procedure was used for the related pairs, except that the sampling population corresponded to the progeny of a parental population and was generated according to Mendelian inheritance. This procedure was repeated 1×10⁶ times for both related and unrelated pairs.

Statistical Analyses and Data Visualization.

Unless otherwise stated, analyses and figures were performed using the RStudio™ version 0.98.1091, R version 3.1.2 and Prism™ version 5.0d software. The Wilcoxon rank sum test was used to compare the polymorphic index distribution of exons and exon-exon junctions, or of MiHA-coding genes and that of genes coding for non-polymorphic MAPs. The gplots package in R was used to perform hierarchical clustering and heatmaps of MiHA genes expression in different AML subtypes. Mean expression of MiHA genes among AML subtypes was compared using ANOVA followed by Tukey's multiple-comparison test.

### Example 2: Identification and Characterization of Human MiHAs

A MiHA is essentially a MAP coded by a genomic region containing an ns-SNP.^{13,21} All human MiHAs discovered to date derive from bi-allelic loci with either two co-dominant alleles or one dominant and one recessive allele.^{21,26} Indeed, an ns-SNP in a MAP-coding sequence will either hinder MAP generation or generate a variant MAP.¹¹ Hence, at the peptidomic level, each allele can be dominant (generate a MAP) or recessive (a null allele that generates no MAP). All MiHAs reported in this work were detected by MS and are therefore coded by dominant alleles. It was reasoned that two features should dictate which of these MiHAs may represent adequate targets for immunotherapy of HCs. First, the usefulness of a MiHA is determined by the allelic frequency of the MiHA-coding ns-SNP. Indeed, in order to be recognized by allogeneic T cells, a MiHA must be present on host cells and absent in donor cells (otherwise, donor T cells would not recognize the MiHA as non-self). This situation is referred to as a “therapeutic mismatch”. The probability to have a therapeutic mismatch is maximal when the allelic frequency of the target MiHA is 0.5 and decreases as the allele frequency approaches the two extremes of 0 and 1.¹⁴ Thus, MiHA having an allele frequency of 0.01 or 0.99 would yield a low frequency of therapeutic mismatch: in the first case, MiHA-positive recipients would be rare whereas in the second case, MiHA-negative donors would be difficult to find. As a rule, only variants with a MAF 0.05 are considered as common and balanced genetic polymorphisms.³³ Thus, all MiHAs coded by loci whose least common (minor) allele had a frequency <0.05 were excluded. Second, the tissue distribution of a MiHA is relevant to both the efficacy and the innocuity of MiHA targeting. For HC immunotherapy, the target MiHA must be expressed in hematopoietic cells (including HC cells) but should not be ubiquitously expressed by host tissues.

Proteogenomic analyses were performed on B lymphoblastoid cell lines (BLCLs) from 18 individuals (9 females and 9 males) expressing at least one of the following alleles: HLA-A*01:01, HLA-A*03:01, HLA-A*11:01, HLA-A*24:02, HLA-A*29:02, HLA-A*32:01, HLA-B*07:02, HLA-B*08:01, HLA-B*13:02, HLA-B*14:02, HLA-B*15:01, HLA-B*18:01, HLA-B*27:05, HLA-B*35:01, HLA-B*40:01, HLA-B*44:02 and HLA-B*57:01. Whole exome and transcriptome sequencing was performed for each cell line in order to identify ns-SNPs and then in silico translated the genomic sequences to create personalized proteomes. Each proteome was subsequently used as a reference to sequence the individual-specific repertoire of MAPs by high-throughput MS.²⁶ Several MiHA candidates generated by ns-SNPs were identified by MS. However, most of these ns-SNPs were of limited clinical interest because they were rare variants with a MAF <0.05. Further analyses focused on common variants, with a MAF 0.05.³³ After filtering and manual MS validation, several high-frequency MiHAs were identified (Table II).

TABLE II
Features of MIHAs identified in the studies described herein

| | | | | | SEQ ID |
|---|---|---|---|---|---|
| Name | Sequence¹ | HLA | SNP_ID | Ensembl gene ID | NO: |
| RASSF1-1A/S | A/SEIEQKIKEY | B18.01 | rs2073498 | ENSG00000068028 | 4-6 |
| RASSF1-1A/S | A/SEIEQKIKEY | B40.01 | rs2073498 | ENSG00000068028 | 4-6 |
| RASSF1-1A/S | A/SEIEQKIKEY | B44.02 | rs2073498 | ENSG00000068028 | 4-6 |
| LTA-1P/H | AAQTARQP/HPK | A11.01 | rs2229092 | ENSG00000226979 | 7-9 |
| CCDC34-1E/A | AE/AIQEKKEI | B44.02 | rs17244028 | ENSG00000109881 | 12-14 |
| TRAPPC5-1S/A | AELQS/ARLAA | B44.02 | rs6952 | ENSG00000181029 | 15-17 |
| HIST1H1C-1A/V | APPAEKA/VPV | B07.02 | rs2230653 | ENSG00000187837 | 18-20 |
| ZC3H12D-1P/Q | APREP/QFAHSL | B07.02 | rs150937045 | ENSG00000178199 | 21-23 |
| MKI67-3S/N | APRES/NAQAI | B07.02 | rs10082533 | ENSG00000148773 | 24-26 |
| PDLIM5-1F/S | APRPFGSVF/S | B07.02 | rs2452600 | ENSG00000163110 | 27-29 |
| RIN3-1R/C | APRR/CPPPPP | B07.02 | rs117068593 | ENSG00000100599 | 30-32 |
| LTA-2P/H | AQTARQP/HPK | A11.01 | rs2229092 | ENSG00000226979 | 33-35 |
| SMARCA5-1Y/* | DRANRFEY/*L | B14.02 | rs11100790 | ENSG00000153147 | 36-38 |
| OAS3-1K/R/M/T | DRFVARK/R/M/TL | B14.02 | rs1859330 | ENSG00000111331 | 39-43 |
| HJURP-1E/G | EE/GRGENTSY | B44.02 | rs10511 | ENSG00000123485 | 44-46 |
| TESPA1-1E/K | EE/KEQSQSRW | B44.02 | rs997173 | ENSG00000135426 | 68-70 |
| CPOX-2N/H | EEADGN/HKQWW | B44.02 | rs1131857 | ENSG00000080819 | 47-49 |
| PREX1-1H/Q | EEALGLYH/QW | B44.02 | rs41283558 | ENSG00000124126 | 50-52 |
| MCPH1-R/I | EEINLQR/INI | B40.01 | rs2083914 | ENSG00000147316 | 53-55 |
| MCPH1-R/I | EEINLQR/INI | B44.02 | rs2083914 | ENSG00000147316 | 53-55 |
| BLM-3V/I | EEIPV/ISSHY | A29.02 | rs7167216 | ENSG00000197299 | 56-58 |
| BLM-3V/I | EEIPV/ISSHY | B18.01 | rs7167216 | ENSG00000197299 | 56-58 |
| BLM-3V/I | EEIPV/ISSHY | B44.02 | rs7167216 | ENSG00000197299 | 56-58 |
| BLM-2V/I | EEIPV/ISSHYF | B40.01 | rs7167216 | ENSG00000197299 | 59-61 |
| BLM-2V/I | EEIPV/ISSHYF | B44.02 | rs7167216 | ENSG00000197299 | 59-61 |
| MKI67-1G/S | EELLAVG/SKF | B40.01 | rs2152143 | ENSG00000148773 | 62-64 |
| MKI67-1G/S | EELLAVG/SKF | B44.02 | rs2152143 | ENSG00000148773 | 62-64 |
| MKI67-1G/S | EELLAVS/GKF | B44.02 | rs2152143 | ENSG00000148773 | 62-64 |
| MIIP-2K/E | EESAVPE/KRSW | B44.02 | rs2295283 | ENSG00000116691 | 65-67 |
| MIIP-2K/E | EESAVPK/ERSW | B44.02 | rs2295283 | ENSG00000116691 | 65-67 |
| TRAPPC5-2A/S | ELQA/SRLAAL | B08.01 | rs6952 | ENSG00000181029 | 71-73 |
| HJURP-1S/F | EPQGS/FGRQGNSL | B07.02 | rs12582 | ENSG00000123485 | 74-76 |
| HMMR-4R/C | ESKIR/CVLL | B08.01 | rs299284 | ENSG00000072571 | 77-79 |
| LILRB4-1G/D | G/DPRPSPTRSV | B07.02 | rs731170 | ENSG00000186818 | 80-82 |
| MKI67-2D/G | GED/GKGIKAL | B40.01 | rs10082391 | ENSG00000148773 | 83-85 |
| MKI67-2D/G | GED/GKGIKAL | B44.02 | rs10082391 | ENSG00000148773 | 83-85 |
| IL4R-1A/E | GRA/EGIVARL | B27.05 | rs1805011 | ENSG00000077238 | 86-88 |
| NUP153-1I/V | GTLSPSLGNSSI/VLK | A11.01 | rs2228375 | ENSG00000124789 | 89-91 |
| RNF213-1L/I | HRVYLVRKL/I | B27.05 | rs62077764 | ENSG00000173821 | 92-94 |
| RNF213-2V/L | IYPQV/LLHSL | A24.02 | rs35332090 | ENSG00000173821 | 95-97 |
| BCL2A1-3G/D | KEFEDD/GIINW | B44.02 | rs3826007 | ENSG00000140379 | 98-100 |
| (ACC-2D) | | | | | |
| BCL2A1-3G/D | KEFEDG/DIINW | B18.01 | rs3826007 | ENSG00000140379 | 98-100 |
| BCL2A1-3G/D | KEFEDG/DIINW | B40.01 | rs3826007 | ENSG00000140379 | 98-100 |
| BCL2A1-3G/D | KEFEDG/DIINW | B44.02 | rs3826007 | ENSG00000140379 | 98-100 |
| BCL2A1-3G/D | KEFEDG/DIINW | B57.01 | rs3826007 | ENSG00000140379 | 98-100 |
| SMC4-1N/S | KEINEKSN/SIL | B40.01 | rs33999879 | ENSG00000113810 | 101-103 |
| 5MC4-1N/S | KEINEKSN/SIL | B44.02 | rs33999879 | ENSG00000113810 | 101-103 |
| CRTAM-1A/G | KLYSEA/GKTK | A03.01 | rs1916036 | ENSG00000109943 | 104-106 |
| SP110-1R/W/G | KRVGASYER/W/G | B27.05 | rs1129411 | ENG00000135899 | 107-110 |
| SP100-1M/T | KVKTSLNEQM/TY | B15.01 | rs836237 | ENSG00000067066 | 111-113 |
| CYBA-1Y/H | KY/HMTAVVKL | A24.02 | rs4673 | ENSG00000051523 | 114-116 |
| CYBA-2Y/H | KY/HMTAVVKLF | A24.02 | rs4673 | ENSG00000051523 | 117-119 |
| CYBA-2Y/H | KY/HMTAVVKLF | B15.01 | rs4673 | ENSG00000051523 | 117-119 |
| DNMT1-1H/R | LENGAH/RAY | B18.01 | rs16999593 | ENSG00000130816 | 120-122 |
| LTA-3C/R | LPRVC/RGTTL | B07.02 | rs2229094 | ENSG00000226979 | 123-125 |
| MKI67-4L/I | LPSKRVSL/I | B07.02 | rs997983 | ENSG00000148773 | 126-128 |
| TRAF3IP3-1Q/H | LRIQ/HQREQL | B14.02 | rs2076150 | ENSG00000009790 | 129-131 |
| USP15-1T/I | MPSHLRNT/ILL | B07.02 | rs11174420 | ENSG00000135655 | 132-134 |
| USP15-2T/I | MPSHLRNT/ILLM | B07.02 | rs11174420 | ENSG00000135655 | 135-137 |
| HY-UTY-2 | NESNTQKTY or | B44.02 | Y-linked | ENSG00000183878 | 10 |
| | absence² | | | | |
| PXK-1R/K | NSEEHSAR/KY | A01.01 | rs56384862 | ENSG00000168297 | 138-140 |
| H3F3C-1H/P | PH/PRYRPGTVAL | B07.02 | rs3759295 | ENSG00000188375 | 141-143 |
| TGFB1-1P/L | PPSGLRLLP/LL | B07.02 | rs1800470 | ENSG00000105329 | 144-146 |
| MIS18BP1-1E/D | QE/DLIGKKEY | B18.01 | rs34101857 | ENSG00000129534 | 147-149 |
| MIS18BP1-1E/D | QE/DLIGKKEY | B44.02 | rs34101857 | ENSG00000129534 | 147-149 |
| ZWINT-1G/R | QELDG/RVFQKL | B18.01 | rs2241666 | ENSG00000122952 | 155-157 |
| ZWINT-1G/R | QELDG/RVFQKL | B44.02 | rs2241666 | ENSG00000122952 | 155-157 |
| ZWINT-1G/R | QELDR/GVFQKL | B40.01 | rs2241666 | ENSG00000122952 | 155-157 |
| ZWINT-1G/R | QELDR/GVFQKL | B44.02 | rs2241666 | ENSG00000122952 | 155-157 |
| CENPF- | QEN/DIQ/HNLQL | B44.02 | rs3748693 | ENSG00000117724 | 150-154 |
| 1NQ/DQ/NH/DH | | | | | |
| CENPF- | QEN/DIQ/HNLQL | B44.02 | rs3748692 | EN5G00000117724 | 150-154 |
| 1NQ/DQ/NH/DH | | | | | |
| TROAP-1R/G | QENQDPR/GRW | B44.02 | rs8285 | ENSG00000135451 | 158-160 |
| GBP4-1Y/N | QERSFQEY/N | B18.01 | rs655260 | ENSG00000162654 | 161-163 |
| INDEL-PPTC7-1 | QTDPRAGGGGGGDY | A01.01 | rs151075597 | ENSG00000196850 | 11 |
| | or absence | | | | |
| CENPM-1R/* | R/*VWDLPGVLK | A11.01 | rs5758511 | ENSG00000100162 | 1-3 |
| (PANE1) | | | | | |
| CENPM-1R/* | R/*VWDLPGVLK | B27.05 | rs5758511 | ENSG00000100162 | 1-3 |
| (PANE1) | | | | | |
| APOBEC3H- | R/GIFASRLYY | A32.01 | rs139297 | ENSG00000100298 | 164-166 |
| 2R/G | | | | | |
| NUSAP1-1T/A | RANLRAT/AKL | B07.02 | rs7178634 | ENSG00000137804 | 167-170 |
| NUSAP1-2T/N | RANLRAT/NKL | B07.02 | rs7178777 | ENSG00000137804 | 167-170 |
| FBXO7-1G/E | RPPG/EGSGPL | B07.02 | rs9621461 | ENSG00000100225 | 171-173 |
| FBXO7- | RPPG/EGSGPLL/H/R/P | B07.02 | rs8137714 | ENSG00000100225 | 174-182 |
| 2GL/EL/GH/EH/ | | | | | |
| GR/ER/GP/EP | | | | | |
| FBXO7- | RPPG/EGSGPLL/H/R/P | B07.02 | rs9621461 | ENSG00000100225 | 174-182 |
| 2GL/EL/GH/EH/ | | | | | |
| GR/ER/GP/EP | | | | | |
| KDM6B-1P/S | RPPPP/SPAWL | B07.02 | rs62059713 | ENSG00000132510 | 183-185 |
| TCL1A-1V/I | RREDV/IVLGR | B27.05 | rs17093294 | ENSG00000100721 | 186-188 |
| RNF213-3A/T | RTA/TDNFDDILK | A11.01 | rs61359568 | ENSG00000173821 | 189-191 |
| RASSF1-1A/S | S/AEIEQKIKEY | B44.02 | rs2073498 | ENSG00000068028 | 4-6 |
| ELF1-1S/T | S/TVLKPGNSK | A11.01 | rs1056820 | ENSG00000120690 | 192-194 |
| MIIP-1K/E | SEESAVPE/KRSW | B44.02 | rs2295283 | ENSG00000116691 | 195-197 |
| MIIP-1K/E | SEESAVPK/ERSW | B44.02 | rs2295283 | ENSG00000116691 | 195-197 |
| HMMR-3R/C | SESKIR/CVLL | B40.01 | rs299284 | ENSG00000072571 | 198-200 |
| HMMR-3R/C | SESKIR/CVLL | B44.02 | rs299284 | ENSG00000072571 | 198-200 |
| GTSE1-1D/E | SPD/ESSTPKL | B07.02 | rs6008684 | ENSG00000075218 | 201-203 |
| OSCAR-1N/K | SPRGN/KLPLLL | B07.02 | rs1657535 | ENSG00000170909 | 204-206 |
| RASSF1-2A/S | SQA/SEIEQKI | B13.02 | rs2073498 | ENSG00000068028 | 207-209 |
| BCL2A1-2K/N | SRVLQN/KVAF | B27.05 | rs1138358 | ENSG00000140379 | 210-212 |
| ZBTB1-1T/N | SVSKLST/NPK | A11.01 | rs45512391 | ENSG00000126804 | 213-215 |
| C17orf53-1T/P | T/PARPQSSAL | B07.02 | rs227584 | ENSG00000125319 | 216-218 |
| ELF1-1S/T | T/SVLKPGNSK | A11.01 | rs1056820 | ENSG00000120690 | 192-194 |
| MK167-5A/V | TAKQKLDPA/V | B08.01 | rs45549235 | ENSG00000148773 | 219-221 |
| BCLAF1-1N/S | TLN/SERFTSY | B15.01 | rs7381749 | ENSG00000029363 | 222-224 |
| MKI67-6L/V | TPRNTYKMTSL/V | B07.02 | rs2240 | ENSG00000148773 | 225-227 |
| WIPF1-1L/P | TPRPIQSSL/P | B07.02 | rs4972450 | ENSG00000115935 | 228-230 |
| DDX20-1R/S | TPVDDR/SSL | B35.01 | rs197414 | ENSG00000064703 | 231-233 |
| MCM7-1R/S | TQR/SPADVIF | B15.01 | rs1130958 | ENSG00000166508 | 234-236 |
| PRC1-1Y/C | TVY/CHSPVSR | A03.01 | rs12911192 | ENSG00000198901 | 237-239 |
| CPOX-1N/H | VEEADGN/HKQW | B44.02 | rs1131857 | ENSG00000080819 | 240-242 |
| IFIH1-1H/R | VYNNIMRH/RYL | A24.02 | rs10930046 | ENSG00000115267 | 243-245 |
| OAS3-2S/R | YPRAGS/RKPP | B07.02 | rs2285933 | ENSG00000111331 | 246-248 |
| UHRF1BP1L- | YTDSSSI/VLNY | A01.01 | rs60592197 | ENSG00000111647 | 249-251 |
| 1I/V | | | | | |

¹The residues in bold and separated by “/” indicate the amino acid variation(s) present in the MiHA.
^{a}The genes from which this MiHA is derived is located on chromosome Y. Accordingly, this MiHa is present in male but absent in female individuals.
^{b}Deletion mutation (CGC codon) resulting in absence of the MiHA (SNP rs151075597).

Tables III-a to III-q below depict the MiHA identified herein, sorted by HLA alleles. Some of the MiHAs identified herein were previously reported for other HLA alleles, as indicated.

TABLE III-a
HLA.A01.01

| | | HLA | Previously |
|---|---|---|---|
| | | (present | reported |
| Name | Sequence¹ | study) | for |
| PXK-1R/K | NSEEHSAR/KY | HLA.A01.01 | — |
| INDEL-PPTC7-1 | QTDPRAGGGGG | HLA.A01.01 | — |
| | GDY or | | |
| | absence | | |
| UHRF1BP1L-1I/V | YTDSSSI/VLNY | HLA.A01.01 | — |

TABLE III-b
HLA.A03.01

| | | HLA (present | Previously |
|---|---|---|---|
| Name | Sequence¹ | study) | reported for |
| CRTAM-1A/G | KLYSEA/GKTK | HLA.A03.01 | — |
| PRC1-1Y/C | TVY/CHSPVSR | HLA.A03.01 | — |

TABLE III-c
HLA.A11.01

| | | HLA | Previously |
|---|---|---|---|
| | | (present | reported |
| Name | Sequence¹ | study) | for |
| LTA-1P/H | AAQTARQP/HPK | HLA.A11.01 | — |
| LTA-2P/H | AQTARQP/HPK | HLA.A11.01 | — |
| NUP153-1I/V | GTLSPSLGNSSI/VL | HLA.A11.01 | — |
| | K | | |
| CENPM-1R/* | R/*VWDLPGVLK | HLA.A11.01 | HLA.A03 |
| (PANE1) | | | |
| RNF213-3A/T | RTA/TDNFDDILK | HLA.A11.01 | — |
| ELF1-1S/T | S/TVLKPGNSK | HLA.A11.01 | — |
| ZBTB1-1T/N | SVSKLST/NPK | HLA.A11.01 | — |
| ELF1-1S/T | T/SVLKPGNSK | HLA.A11.01 | — |

TABLE III-d
HLA.A24.02

| | | HLA | Previously |
|---|---|---|---|
| | | (present | reported |
| Name | Sequence¹ | study) | for |
| RNF213-2V/L | IYPQV/LLHSL | HLA.A24.02 | — |
| CYBA-1Y/H | KY/HMTAVVKL | HLA.A24.02 | — |
| CYBA-2Y/H | KY/HMTAVVKLF | HLA.A24.02 | — |
| IFIH1-1H/R | VYNNIMRH/RYL | HLA.A24.02 | — |

TABLE III-e
HLA.A29.02

| | | HLA | Previously |
|---|---|---|---|
| | | (present | reported |
| Name | Sequence¹ | study) | for |
| BLM-3V/I | EEIPV/ISSHY | HLA.A29.02 | HLA.B44.02 |

TABLE III-f
HLA.A32.01

| | | HLA | Previously |
|---|---|---|---|
| | | (present | reported |
| Name | Sequence¹ | study) | for |
| APOBEC3H- | R/GIFASRLYY | HLA.A32.01 | — |
| 2R/G | | | |

TABLE III-g
HLA.B07.02

| | | HLA | Previously |
|---|---|---|---|
| | | (present | reported |
| Name | Sequence¹ | study) | for |
| HIST1H1C-1A/V | APPAEKA/VPV | HLA.B07.02 | — |
| ZC3H12D-1P/Q | APREP/QFAHSL | HLA.B07.02 | — |
| MKI67-3S/N | APRES/NAQAI | HLA.B07.02 | — |
| PDLIM5-1F/S | APRPFGSVF/S | HLA.B07.02 | — |
| RIN3-1R/C | APRR/CPPPPP | HLA.B07.02 | — |
| HJURP-1S/F | EPQGS/FGRQGNSL | HLA.B07.02 | — |
| LILRB4-1G/D | G/DPRPSPTRSV | HLA.B07.02 | — |
| LTA-3C/R | LPRVC/RGTTL | HLA.B07.02 | — |
| MKI67-4L/I | LPSKRVSL/I | HLA.B07.02 | — |
| USP15-1T/I | MPSHLRNT/ILL | HLA.B07.02 | — |
| USP15-2T/I | MPSHLRNT/ILLM | HLA.B07.02 | — |
| H3F3C-1H/P | PH/PRYRPGTVAL | HLA.B07.02 | — |
| TGFB1-1P/L | PPSGLRLLP/LL | HLA.B07.02 | — |
| NUSAP1-1T/A | RANLRAT/AKL | HLA.B07.02 | — |
| NUSAP1-2T/N | RANLRAT/NKL | HLA.B07.02 | — |
| FBXO7-1G/E | RPPG/EGSGPL | HLA.B07.02 | — |
| FBXO7- | RPPG/EGSGPLL/H/ | HLA.B07.02 | — |
| 2GL/EL/GH/EH/ | R/P | | |
| GR/ER/GP/EP | | | |
| FBXO7- | RPPG/EGSGPLL/H/ | HLA.B07.02 | — |
| 2GL/EL/GH/EH/ | R/P | | |
| GR/ER/GP/EP | | | |
| KDM6B-1P/S | RPPPP/SPAWL | HLA.B07.02 | — |
| GTSE1-1D/E | SPD/ESSTPKL | HLA.B07.02 | — |
| OSCAR-1N/K | SPRGN/KLPLLL | HLA.B07.02 | — |
| C17orf53-1T/P | T/PARPQSSAL | HLA.B07.02 | — |
| MKI67-6L/V | TPRNTYKMTSL/V | HLA.B07.02 | — |
| WIPF1-1L/P | TPRPIQSSL/P | HLA.B07.02 | — |
| OA53-2S/R | YPRAGS/RKPP | HLA.B07.02 | — |

TABLE III-h
HLA.B08.01

| | | HLA (present | Previously |
|---|---|---|---|
| Name | Sequence¹ | study) | reported for |
| TRAPPC5-2A/S | ELQA/SRLAAL | HLA.B08.01 | — |
| HMMR-4R/C | ESKIR/CVLL | HLA.B08.01 | — |
| MKI67-5A/V | TAKQKLDPA/V | HLA.B08.01 | — |

TABLE III-i
HLA.B13.02

| | | HLA (present | Previously |
|---|---|---|---|
| Name | Sequence¹ | study) | reported for |
| RASSF1-2A/S | SQA/SEIEQKI | HLA.B13.02 | HLA.A02.01 |

TABLE III-j
HLA.B14.02

| | | HLA | Previously |
|---|---|---|---|
| | | (present | reported |
| Name | Sequence¹ | study) | for |
| SMARCA5-1Y/* | DRANRFEY/*L | HLA.B14.02 | — |
| OAS3-1 K/R/M/T | DRFVARK/R/M/TL | HLA.B14.02 | — |
| TRAF3IP3-1Q/H | LRIQ/HQREQL | HLA.B14.02 | — |

TABLE III-k
HLA.B15.01

| | | HLA (present | Previously |
|---|---|---|---|
| Name | Sequence¹ | study) | reported for |
| SP100-1M/T | KVKTSLNEQM/TY | HLA.B15.01 | — |
| CYBA-2Y/H | KY/HMTAVVKLF | HLA.B15.01 | — |
| BCLAF1-1N/S | TLN/SERFTSY | HLA.B15.01 | — |
| MCM7-1R/S | TQR/SPADVIF | HLA.B15.01 | — |

TABLE III-l
HLA.B18.01

| | | HLA | Previously |
|---|---|---|---|
| | | (present | reported |
| Name | Sequence¹ | study) | for |
| RASSF1-1A/S | A/SEIEQKIKEY | HLA.B18.01 | HLA.B44.03 |
| BLM-3V/I | EEIPV/ISSHY | HLA.B18.01 | HLA.B44.03 |
| BCL2A1-3G/D | KEFEDG/DIINW | HLA.B18.01 | HLA.B44.03 |
| DNMT1-1H/R | LENGAH/RAY | HLA.B18.01 | — |
| MIS18BP1-1E/D | QE/DLIGKKEY | HLA.B18.01 | HLA.B44.03 |
| ZWINT-1G/R | QELDG/RVFQKL | HLA.B18.01 | HLA.B44.03 |
| GBP4-1Y/N | QERSFQEY/N | HLA.B18.01 | — |

TABLE III-m
HLA.B27.05

| | | HLA (present | Previously |
|---|---|---|---|
| Name | Sequence¹ | study) | reported for |
| IL4R-1A/E | GRA/EGIVARL | HLA.B27.05 | — |
| RNF213-1L/I | HRVYLVRKL/I | HLA.B27.05 | — |
| SP110-1R/W/G | KRVGASYER/W/G | HLA.B27.05 | — |
| CENPM-1R/* | R/*VWDLPGVLK | HLA.B27.05 | HLA.A03 |
| (PANE1) | | | |
| TCL1A-1V/I | RREDV/IVLGR | HLA.B27.05 | — |
| BCL2A1-2K/N | SRVLQN/KVAF | HLA.B27.05 | — |

TABLE III-n
HLA.B35.01

| | | HLA | Previously |
|---|---|---|---|
| | | (present | reported |
| Name | Sequence¹ | study) | for |
| DDX20-1R/S | TPVDDR/SSL | HLA.B35.01 | |

TABLE III-o
HLA.B40.01

| | | HLA | Previously |
|---|---|---|---|
| | | (present | reported |
| Name | Sequence¹ | study) | for |
| RASSF1-1A/S | A/SEIEQKIKEY | HLA.B40.01 | HLA.B44.03 |
| MCPH1-R/I | EEINLQR/INI | HLA.B40.01 | HLA.B44.03 |
| BLM-2V/I | EEIPV/ISSHYF | HLA.B40.01 | HLA.B44.03 |
| MKI67-1G/S | EELLAVG/SKF | HLA.B40.01 | HLA.B44.03 |
| MKI67-2D/G | GED/GKGIKAL | HLA.B40.01 | HLA.B44.03 |
| BCL2A1-3G/D | KEFEDG/DIINW | HLA.B40.01 | HLA.B44.03 |
| SMC4-1N/S | KEINEKSN/SIL | HLA.B40.01 | HLA.B44.03 |
| ZWINT-1G/R | QELDR/GVFQKL | HLA.B40.01 | HLA.B44.03 |
| HMMR-3R/C | SESKIR/CVLL | HLA.B40.01 | HLA.B44.03 |

TABLE III-p
HLA.B44.02

| | | HLA (present | Previously |
|---|---|---|---|
| Name | Sequence¹ | study) | reported for |
| RASSF1-1A/S | A/SEIEQKIKEY | HLA.B44.02 | HLA.B44.03 |
| CCDC34-1E/A | AE/AIQEKKEI | HLA.B44.02 | HLA.B44.03 |
| TRAPPC5-1S/A | AELQS/ARLAA | HLA.B44.02 | HLA.B44.03 |
| HJURP-1E/G | EE/GRGENTSY | HLA.B44.02 | HLA.B44.03 |
| TESPA1-1E/K | EE/KEQSQSRW | HLA.B44.02 | HLA.B44.03 |
| CPOX-2N/H | EEADGN/HKQWW | HLA.B44.02 | HLA.B44.03 |
| PREX1-1H/Q | EEALGLYH/QW | HLA.B44.02 | HLA.B44.03 |
| MCPH1-R/I | EEINLQR/INI | HLA.B44.02 | HLA.B44.03 |
| BLM-3V/I | EEIPV/ISSHY | HLA.B44.02 | HLA.B44.03 |
| BLM-2V/I | EEIPV/ISSHYF | HLA.B44.02 | HLA.B44.03 |
| MKI67-1G/S | EELLAVG/SKF | HLA.B44.02 | HLA.B44.03 |
| MKI67-1G/S | EELLAVS/GKF | HLA.B44.02 | HLA.B44.03 |
| MIIP-2K/E | EESAVPE/KRSW | HLA.B44.02 | HLA.B44.03 |
| MIIP-2K/E | EESAVPK/ERSW | HLA.B44.02 | HLA.B44.03 |
| MKI67-2D/G | GED/GKGIKAL | HLA.B44.02 | HLA.B44.03 |
| BCL2A1-3G/D | KEFEDD/GIINW | HLA.B44.02 | HLA.B44.03 |
| (ACC-2D) | | | |
| BCL2A1-3G/D | KEFEDG/DIINW | HLA.B44.02 | HLA.B44.03 |
| SMC4-1N/S | KEINEKSN/SIL | HLA.B44.02 | HLA.B44.03 |
| HY-UTY-2 | NESNTQKTY or | HLA.B44.02 | HLA.B44.03 |
| | absence² | | |
| MIS18BP1- | QE/DLIGKKEY | HLA.B44.02 | HLA.B44.03 |
| 1E/D | | | |
| ZWINT-1G/R | QELDG/RVFQKL | HLA.B44.02 | HLA.B44.03 |
| ZWINT-1G/R | QELDR/GVFQKL | HLA.B44.02 | HLA.B44.03 |
| CENPF- | QEN/DIQ/HNLQL | HLA.B44.02 | HLA.B44.03 |
| 1NQ/DQ/NH/ | | | |
| DH | | | |
| CENPF- | QEN/DIQ/HNLQL | HLA.B44.02 | HLA.B44.03 |
| 1NQ/DQ/NH/ | | | |
| DH | | | |
| TROAP-1R/G | QENQDPR/GRW | HLA.B44.02 | HLA.B44.03 |
| RASSF1-1A/S | S/AEIEQKIKEY | HLA.B44.02 | — |
| MIIP-1K/E | SEESAVPE/KRSW | HLA.B44.02 | HLA.B44.03 |
| MIIP-1K/E | SEESAVPK/ERSW | HLA.B44.02 | HLA.B44.03 |
| HMMR-3R/C | SESKIR/CVLL | HLA.B44.02 | HLA.B44.03 |
| CPOX-1N/H | VEEADGN/HKQW | HLA.B44.02 | HLA.B44.03 |

TABLE III-q
HLA.B57.01

| | | HLA | Previously |
|---|---|---|---|
| | | (present | reported |
| Name | Sequence¹ | study) | for |
| BCL2A1-3G/D | KEFEDG/DIINW | HLA.B57.01 | HLA.B44.03 |

### Example 3: The MiHAs Identified are Coded by Genes Preferentially Expressed in Hematopoietic Cells

It was assumed that, for hematopoietic cancer (HC) immunotherapy, optimal MiHAs should be expressed on hematopoietic cells, including the target HC cells, but should ideally not be ubiquitously expressed. Indeed, ubiquitous expression decreases the efficacy of immunotherapy by causing exhaustion of MiHA-specific T cells and entails the risk of toxicity toward normal host epithelial cells (Graft-versus-Host-Disease, GvHD). Since the abundance of a MAP shows a good correlation with the abundance of its source transcript,^{22,38-40} and RNA-Seq is currently the most accurate method for evaluation of transcript abundance, the expression level of MiHA-coding transcripts was evaluated by RNA-Seq. No RNA-Seq data are available for purified primary epithelial cells from all anatomic sites, but this information is available for entire tissues and organs. Publicly available RNA-Seq data on 27 human tissues from different individuals³⁰ were therefore used to assess the expression profile of genes coding the MiHAs presented by the HLA-A*01:01, HLA-A*03:01, HLA-A*11:01, HLA-A*24:02, HLA-A*29:02, HLA-A*32:01, HLA-B*07:02, HLA-B*08:01, HLA-B*13:02, HLA-B*14:02, HLA-B*15:01, HLA-B*18:01, HLA-B*27:05, HLA-B*35:01, HLA-B*40:01, HLA-B*44:02 and/or HLA-B*57:01 allele. To evaluate the relative expression of MiHA-coding genes in hematopoietic vs. epithelial cells, RNA-Seq data obtained from bone marrow vs. skin cells were used. Skin cells are not a pure population of epithelial cells (they contain cells of monocytic and dendritic cell lineage), but are nevertheless highly enriched in epithelial relative to hematopoietic cells. As a criterion for preferential expression in hematopoietic cells, an expression ratio 2 in the bone marrow relative to the skin was used.

Acute myeloid leukemia (AML) is the most common indication for AHCT according to the Center for International Blood and Marrow Transplant Research (CIBMTR, http://www.cibmtr.org). The expression of genes coding the MiHAs identified herein in AML cells was thus analyzed using RNA-Seq data from 179 AML samples available from The Cancer Genome Atlas (TCGA). The predicted binding affinity of the MiHA identified herein was also determined using NetMHC⁵⁸⁻⁶⁰. Results from these analyses are depicted in Table IV.

TABLE IV
Selected features of the MiHAs described herein.

| | MAF | | IC₅₀ | BM/skin | AMLs |
|---|---|---|---|---|---|
| MiHA Name | Global/EA | HLA | (nM) | ratio | (RPKM) |
| RASSF1-1A/S | 0.08/0.10 | HLA.B18.01 | 788 | 2.54 | 49.38 |
| RASSF1-1A/S | 0.08/0.10 | HLA.B40.01 | 3015 | 2.54 | 49.38 |
| RASSF1-1A/S | 0.08/0.10 | HLA.B44.02 | 34 | 2.54 | 49.38 |
| LTA-1P/H | 0.03/0.07 | HLA.A11.01 | 95 | 11.00 | 0.37 |
| CCDC34-1E/A | 0.20/0.35 | HLA.B44.02 | 35 | 2.14 | 3.14 |
| TRAPPC5-1S/A | 0.34/0.27 | HLA.B44.02 | 737 | 2.59 | 30.74 |
| HIST1H1C-1A/V | 0.19/0.02 | HLA.B07.02 | 222 | 22.47 | 11.70 |
| ZC3H12D-1P/Q | 0.07/N.A. | HLA.B07.02 | 14 | 2.37 | 4.03 |
| MKI67-3S/N | 0.22/0.17 | HLA.B07.02 | 10 | 5.16 | 19.89 |
| PDLIM5-1F/S | 0.28/0.31 | HLA.B07.02 | 7 | 2.00 | 5.47 |
| RIN3-1R/C | 0.08/0.20 | HLA.B07.02 | 147 | 15.58 | 32.22 |
| LTA-2P/H | 0.03/0.07 | HLA.A11.01 | 76 | 11.00 | 0.37 |
| SMARCA5-1Y/* | 0.32/0.19 | HLA.B14.02 | 389 | 2.05 | 35.80 |
| OAS3-1K/R/M/T | 0.34/0.36 | HLA.B14.02 | 1292 | 3.06 | 8.60 |
| HJURP-1E/G | 0.18/0.10 | HLA.B44.02 | 40 | 9.49 | 7.48 |
| TESPA1-1E/K | 0.25/0.07 | HLA.B44.02 | 29 | 5.49 | 21.37 |
| CPOX-2N/H | 0.24/0.13 | HLA.B44.02 | 30 | 2.06 | 13.41 |
| PREX1-1H/Q | 0.14/0.19 | HLA.B44.02 | 30 | 8.24 | 41.48 |
| MCPH1-R/I | 0.08/0.15 | HLA.B40.01 | 3956 | 2.09 | 6.09 |
| MCPH1-R/I | 0.08/0.15 | HLA.B44.02 | 43 | 2.09 | 6.09 |
| BLM-3V/I | 0.07/0.07 | HLA.A29.02 | 3152 | 18.27 | 10.41 |
| BLM-3V/I | 0.07/0.07 | HLA.B18.01 | 74 | 18.27 | 10.41 |
| BLM-3V/I | 0.07/0.07 | HLA.B44.02 | 32 | 18.27 | 10.41 |
| BLM-2V/I | 0.07/0.07 | HLA.B40.01 | 1551 | 9.01 | 10.41 |
| BLM-2V/I | 0.07/0.70 | HLA.B44.02 | 868 | 9.01 | 10.41 |
| MKI67-1G/S | 0.21/0.25 | HLA.B40.01 | 2672 | 4.27 | 19.89 |
| MKI67-1G/S | 0.21/0.25 | HLA.B44.02 | 115 | 4.27 | 19.89 |
| MKI67-1G/S | 0.21/0.25 | HLA.B44.02 | 2833 | 4.27 | 19.89 |
| MIIP-2K/E | 0.34/0.29 | HLA.B44.02 | 23 | 2.69 | 15.83 |
| MIIP-2K/E | 0.34/0.29 | HLA.B44.02 | 16 | 2.69 | 15.83 |
| TRAPPC5-2A/S | 0.34/0.27 | HLA.B08.01 | 22 | 2.07 | 30.74 |
| CENPF-2L/S | 0.10/0.05 | HLA.B08.01 | 13 | 2.54 | 10.85 |
| HJURP-1S/F | 0.18/0.10 | HLA.B07.02 | 43 | 9.49 | 7.48 |
| HMMR-4R/C | 0.08/0.12 | HLA.B08.01 | 2177 | 3.52 | 7.33 |
| LILRB4-1G/D | 0.35/0.31 | HLA.B07.02 | 26 | 25.52 | 2.97 |
| MKI67-2D/G | 0.22/0.17 | HLA.B40.01 | 16 | 4.27 | 19.89 |
| MKI67-2D/G | 0.22/0.17 | HLA.B44.02 | 4473 | 4.27 | 19.89 |
| IL4R-1A/E | 0.22/0.11 | HLA.B27.05 | 52 | 2.66 | 15.09 |
| NUP153-1I/V | 0.14/0.29 | HLA.A11.01 | 12 | 2.42 | 28.38 |
| RNF213-1L/l | 0.04/0.07 | HLA.B27.05 | 45 | 2.28 | 36.60 |
| RNF213-2V/L | 0.13/0.11 | HLA.A24.02 | 15 | 2.28 | 36.60 |
| BCL2A1-3G/D (ACC-2D) | 0.19/0.25 | HLA.B44.02 | 72 | 259.40 | 9.83 |
| BCL2A1-3G/D | 0.19/0.25 | HLA.B18.01 | 950 | 292.97 | 9.83 |
| BCL2A1-3G/D | 0.19/0.25 | HLA.B40.01 | 1545 | 292.97 | 9.83 |
| BCL2A1-3G/D | 0.19/0.25 | HLA.B44.02 | 48 | 292.97 | 9.83 |
| BCL2A1-3G/D | 0.19/0.25 | HLA.B57.01 | 3036 | 292.97 | 9.83 |
| SMC4-1N/S | 0.05/0.05 | HLA.B40.01 | 19 | 3.49 | 42.29 |
| SMC4-1N/S | 0.05/0.05 | HLA.B44.02 | 940 | 3.49 | 42.29 |
| CRTAM-1A/G | 0.09/0.03 | HLA.A03.01 | 21 | 81.00 | 0.70 |
| SP110-1R/W/G | 0.06/0.12 | HLA.B27.05 | 168 | 2.66 | 23.59 |
| SP100-1M/T | 0.27/0.15 | HLA.B15.01 | 266 | 3.82 | 23.41 |
| CYBA-1Y/H | 0.3/0.34 | HLA.A24.02 | 30 | 23.17 | 54.37 |
| CYBA-2Y/H | 0.30/0.34 | HLA.A24.02 | 10 | 23.17 | 54.37 |
| CYBA-2Y/H | 0.30/0.34 | HLA.B15.01 | 1913 | 23.17 | 54.37 |
| DNMT1-1H/R | 0.06/0.00 | HLA.B18.01 | 57 | 2.52 | 34.42 |
| LTA-3C/R | 0.27/0.27 | HLA.B07.02 | 8 | 11.00 | 0.372 |
| MKI67-4L/I | 0.10/0.09 | HLA.B07.02 | 107 | 5.16 | 19.89 |
| TRAF3IP3-1Q/H | 0.37/0.22 | HLA.B14.02 | 1529 | 8.07 | 31.03 |
| USP15-1T/I | 0.30/0.31 | HLA.B07.02 | 28 | 3.64 | 34.04 |
| USP15-2T/I | 0.30/0.31 | HLA.B07.02 | 18 | 3.64 | 34.04 |
| HY-UTY-2 | N.A./0.50 | HLA.B44.02 | 28 | 4.13 | 0.16 |
| PXK-1R/K | 0.20/0.37 | HLA.A01.01 | 12 | 3.63 | 36.86 |
| H3F3C-1H/P | 0.08/0.06 | HLA.B07.02 | 3601 | 5.62 | 0.52 |
| TGFB1-1P/L | 0.44/NA | HLA.B07.02 | 98 | 3.88 | 114.06 |
| MIS18BP1-1E/D | 0.10/0.08 | HLA.B18.01 | 60 | 3.47 | 40.82 |
| MIS18BP1-1E/D | 0.10/0.08 | HLA.B44.02 | 640 | 3.47 | 40.82 |
| ZWINT-1G/R | 0.26/0.37 | HLA.B18.01 | 1301 | 2.80 | 16.48 |
| ZWINT-1G/R | 0.26/0.37 | HLA.B44.02 | 788 | 2.80 | 16.48 |
| ZWINT-1G/R | 0.26/0.37 | HLA.B40.01 | 253 | 2.61 | 16.48 |
| ZWINT-1G/R | 0.26/0.37 | HLA.B44.02 | 92 | 2.61 | 16.48 |
| CENPF-1NQ/DQ/NH/DH | 0.22/0.09 | HLA.B44.02 | 96 | 3.33 | 10.85 |
| CENPF-1NQ/DQ/NH/DH | 0.10/0.05 | HLA.B44.02 | 96 | 3.33 | 10.85 |
| TROAP-1R/G | 0.05/0.01 | HLA.B44.02 | 19 | 4.289 | 8.74 |
| GBP4-1Y/N | 0.29/0.34 | HLA.B18.01 | 184 | 2.24 | 8.15 |
| INDEL-PPTC7-1 | 0.06/0.13 | HLA.A01.01 | 10 | 3.10 | 18.58 |
| CENPM-1R/* (PANE1) | 0.27/0.28 | HLA.A11.01 | 37 | 4.53 | 6.06 |
| CENPM-1R/* (PANE1) | 0.27/0.28 | HLA.B27.05 | 2773 | 4.53 | 6.06 |
| APOBEC3H-2R/G | 0.50/0.46 | HLA.A32.01 | 590 | 13.73 | 1.61 |
| NUSAP1-1T/A | 0.26/0.01 | HLA.B07.02 | 803 | 5.21 | 28.06 |
| NUSAP1-2T/N | 0.26/0.00 | HLA.B07.02 | 803 | 5.21 | 28.06 |
| FBXO7-1G/E | 0.07/0.10 | HLA.B07.02 | 8 | 4.42 | 25.51 |
| FBXO7- | 0.07/0.10 | HLA.B07.02 | 25 | 4.42 | 25.51 |
| 2GL/EL/GH/EH/GR/ER/GP/EP | | | | | |
| FBXO7- | 0.07/0.10 | HLA.B07.02 | 25 | 4.42 | 25.51 |
| 2GL/EL/GH/EH/GR/ER/GP/EP | | | | | |
| KDM6B-1P/S | 0.15/0.14 | HLA.B07.02 | 21 | 2.17 | 17.23 |
| TCL1A-1V/I | 0.05/0.02 | HLA.B27.05 | 274 | 1221.00 | 1.58 |
| RNF213-3A/T | 0.04/0.06 | HLA.A11.01 | 133 | 2.28 | 36.60 |
| RASSF1-1A/S | 0.08/0.10 | HLA.B44.02 | 19 | 2.39 | 49.38 |
| ELF1-1S/T | 0.44/0.32 | HLA.A11.01 | 24 | 3.16 | 86.40 |
| MIIP-1K/E | 0.34/0.29 | HLA.B44.02 | 17 | 2.69 | 15.83 |
| MIIP-1K/E | 0.34/0.29 | HLA.B44.02 | 59 | 2.69 | 15.83 |
| HMMR-3R/C | 0.08/0.12 | HLA.B40.01 | 10 | 3.42 | 7.33 |
| HMMR-3R/C | 0.08/0.12 | HLA.B44.02 | 2535 | 3.42 | 7.33 |
| GTSE1-1D/E | 0.12/0.11 | HLA.B07.02 | 51 | 3.45 | 4.03 |
| OSCAR-1N/K | 0.13/0.03 | HLA.B07.02 | 34 | 10.08 | 12.69 |
| RASSF1-2A/S | 0.08/0.10 | HLA.B13.02 | 1664 | 2.39 | 49.38 |
| BCL2A1-2K/N | 0.43/0.26 | HLA.B27.05 | 616 | 292.97 | 9.83 |
| ZBTB1-1T/N | 0.07/0.10 | HLA.A11.01 | 15 | 2.16 | 16.59 |
| C17orf53-1T/P | 0.43/0.31 | HLA.B07.02 | 23 | 5.81 | 4.42 |
| ELF1-1S/T | 0.44/0.32 | HLA.A11.01 | 34 | 3.16 | 86.40 |
| MKI67-5A/V | 0.06/0.01 | HLA.B08.01 | 375 | 5.16 | 19.89 |
| BCLAF1-1N/S | N.A./0.00 | HLA.B15.01 | 29 | 7.24 | 51.39 |
| MKI67-6L/V | 0.22/0.17 | HLA.B07.02 | 14 | 5.16 | 19.89 |
| WIPF1-1L/P | 0.05/0.04 | HLA.B07.02 | 10 | 6.85 | 46.39 |
| DDX20-1R/S | 0.17/0.13 | HLA.B35.01 | 38 | 2.08 | 7.21 |
| MCM7-1R/S | 0.05/0.05 | HLA.B15.01 | 33 | 3.08 | 56.09 |
| PRC1-1Y/C | 0.03/0.07 | HLA.A03.01 | 86 | 3.98 | 22.00 |
| CPOX-1N/H | 0.24/0.13 | HLA.B44.02 | 44 | 2.06 | 13.41 |
| IFIH1-1H/R | 0.19/0.01 | HLA.A24.02 | 155 | 3.29 | 7.46 |
| OAS3-2S/R | 0.28/0.26 | HLA.B07.02 | 75 | 3.06 | 8.60 |
| UHRF1BP1L-1I/V | 0.05/0.07 | HLA.A01.01 | 6 | 4.35 | 10.92 |

MAF Global/EA: Global MAF reported by dbSNP, and the MAF in European Americans (EA) reported in the Exome Sequencing Project (ESP);
IC₅₀ (nm): the predicted HLA binding affinity (IC₅₀) of the detected MiHA variants according to NetMHC (v.3.4⁵⁸⁻⁶⁰;
BM/skin ratio: relative BM/skin expression of the MiHA-coding transcripts.
AMLs (RPKM): mean MiHA gene expression in primary AML samples (RPKM) obtained from TCGA.

### Example 4: Materials and Methods (for Example 5)

Sample Preparation.

The Epstein-Barr virus (EBV)-transformed B-lymphoblastoid cell line was derived from peripheral blood mononuclear cells as described previously [26]. Cells were grown in RPM11640 containing HEPES and supplemented with 10% heat-inactivated fetal bovine serum, penicillin/streptomycin and L-glutamine and expanded in roller bottles. The cells were then collected, washed with PBS and either used fresh or stored at −80° C. B-ALL specimen used in this study was from an adult male B-ALL patient and was collected and cryopreserved at the Leukemia Cell Bank of Quebec at Maisonneuve-Rosemont Hospital, Montreal. B-ALL cells were expanded in vivo after transplantation in mice as follows. NOD Cg-Prkdc^{scid}/II2rg^{tm1Wjl}/SzJ (NSG) mice were purchased from Jackson Laboratory and bred in a specific pathogen-free animal facility. B-ALL cells were thawed at 37° C., washed and resuspended in RPMI (Life Technologies). A total of 1-2×10⁶ B-ALL cells were transplanted via the tail vein into 8-12-week-old sub-lethally irradiated (250 cGy, 137Cs-gamma source) NSG mice. Mice were sacrificed 30-60 days post-injection when showing signs of disease. Spleens were mechanically dissociated and leukemic cells were isolated by FicoII® gradient. Purity and viability of the samples (usually >90%) were then assessed by flow cytometry. B-ALL cells were identified as human CD45+CD19+.

Flow Cytometry.

Data acquisition was performed on a BD Canto II cytometer (BD Bioscience). The analysis was done with BD FACSDiva® 4.1 software. Antibodies used were anti-human CD45 Pacific Blue (BioLegend 304029), anti-human CD19 PE-Cy7 (BD Bioscience 557835), anti-mouse CD45.1 APC-efluor 730 (eBioscience 47-0453-82) and anti-human HLA-ABC PE (Cedarlane CLHLA-01 PE). The absolute membrane density of MHC I was evaluated by indirect labeling with a purified anti-human HLA-ABC (clone W6/32, eBioscience 14-9983-82), using commercially available QIFIKIT® (Dako) according to the manufacturer's instructions.

Cell Viability Assay.

A 10 μL of resuspended cells (pre- and post-MAE) was added to 10 μL of Trypan blue solution, 0.4%. After mixing, 10 μL was pipetted and transfer into a counting chamber slide. Determination of cell viability was then performed using a countless automated cell counter (Invitrogen).

Peptide Isolation by Immunoprecipitation.

The W6/32 antibodies (BioXcell) were incubated in PBS for 60 minutes at room temperature with PureProteome protein A magnetic beads (Millipore) at a ratio of 1 mg of antibody per mL of slurry. Antibodies were covalently cross-linked to magnetic beads using dimethylpimelidate as described [61]. The beads were stored at 4° C. in PBS pH 7.2 and 0.02% NaN₃. Biological replicates of 2×10⁶, 20×10⁶ and 100×10⁶ cell pellets from both cell types were resuspended in 1 mL PBS pH 7.2 and solubilized by adding 1 mL of detergent buffer containing PBS pH 7.2, 1% (w/v) CHAPS (Sigma) supplemented with Protease inhibitor cocktail (Sigma). After a 60-minute incubation with tumbling at 4° C., samples were spun at 10000 g for 30 minutes at 4° C. Post-nuclear supernatants were transferred into new tubes containing magnetic beads coupled to W6/32 antibodies at a ratio of 10 μg of W6/32 antibody per 1×10⁶ cells. Samples were incubated with tumbling for 180 minutes at 4° C. and placed on a magnet to recover bound MHC I complexes to magnetic beads. Magnetic beads were first washed with 4×1 mL PBS, then with 1×1 mL of 0.1×PBS and finally with 1×1 mL of water. MHC I complexes were eluted from the magnetic beads by acidic treatment using 0.2% trifluoroacetic acid (TFA). To remove any residual magnetic beads, eluates were transferred into 2.0 mL Costar mL Spin-X centrifuge tube filters (0.45 μm, Corning) and spun 2 minutes at 3000 g. Filtrates containing peptides were separated from MHC I subunits (HLA molecules and β-2 macroglobulin) using home-made stage tips packed with twenty 1 mm diameter octadecyl (C-18) solid phase extraction disks (EMPORE). Stage tips were pre-washed first with methanol then with 80% acetonitrile (ACN) in 0.2% TFA and finally with 0.1% formic acid (FA). Samples were loaded onto the stage tips and the peptides were retained on the stage tips while the HLA molecules and β-2 macroglobulin were found in the flow through. Stage tips were washed with 0.1% FA and peptides were eluted with 30% ACN in 0.1% TFA. The peptides were dried using vacuum centrifugation and then stored at −20° C. until MS analysis.

Peptide Isolation by Mild Acid Elution.

Biological replicates of 2×10⁶, 20×10⁶ and 100×10⁶ cells from both cell types were used. Peptides were released by mild acid elution using 1 mL of citrate pH 3.3 buffer for the 2×10⁶ and 20×10⁶ cell samples while 1.5 mL of citrate pH 3.3 buffer was used for the 100×10⁶ cell samples. Samples were then desalted using an HLB cartridge and filtered with a 3,000 Da cut-off column as previously described [38].

Mass Spectrometry and Peptide Sequencing.

Vacuum dried fractions were resuspended in 17 μL of 5% ACN, 0.2% FA and analyzed by LC-MS/MS using an Easy nLC1000 coupled to a Q Exactive HF mass spectrometer (Thermo Fisher Scientific). Peptides were separated on a custom C18 reversed phase column (150 μm i.d.×100 mm, Jupiter Proteo 4 μm, Phenomenex) using a flow rate of 600 nL/min and a linear gradient of 5-30% ACN (0.2% FA) in 56 min, followed by 3.3 min at 80% ACN (0.2% FA). Survey scan (MS1) were acquired with the Orbitrap at a resolving power of 60,000 (at m/z 200) over a scan range of 350-1200 m/z with a target values of 3×10⁶ with a maximum injection time of 100 ms. Mass calibration used an internal lock mass (protonated (Si(CH₃)₂O))₆; m/z 445.120029) and mass accuracy of peptide measurements was within 5 ppm. MS/MS spectra were acquired at higher energy collisional dissociation with a normalized collision energy of 25. Up to twenty precursor ions were accumulated with a precursor isolation window of 1.6 m/z, an advanced gain control (AGC) of 5×10⁴ with a maximum injection time of 50 ms and fragment ions were transferred to the Orbitrap analyzer operating at a resolution of 30,000 at m/z 200.

Peptide Identification and Label-Free Quantification.

Database searches were performed using PEAKS 8 (Bioinformatics Solutions Inc.) Mass tolerances for precursor and fragment ions were set to 10 ppm and 0.02 Da, respectively. Searches were performed without enzyme specificity and with variable modifications for deamidation (N, Q) and Oxidation (M). Subject-specific protein sequence databases that incorporate single amino-acid polymorphism (SAP) detected by RNAseq were generated with a Python script relying on pyGeno (v1.2.9) [62]. Ensembl reference genome release 75 (GRCh37.p13) and 88 (GRCh38.p10) were used for B-LCL and B-ALL cells, respectively. Polymorphisms were called by Casava (Illumina) for B-LCL and FreeBayes [63] for B-ALL. Additionally, for B-ALL, only sequences with expressed transcript were retained. Label-free quantification was performed using PEAKS with mass tolerance of 6 ppm and retention time windows of 0.8 min to compare MHC I peptide abundance across samples. Peaks areas were median normalized only for replicates of the same condition.

Bioinformatic Analyses.

MHC I peptide selection was achieved using the following criteria: peptide false discovery rate was limited to 5%, peptide length between 8-15 residues, and a threshold of top 2% ranked predicted sequences according to NetMHC 4.0. PEAKS result files were processed using Jupyter/IPython notebooks (v1.0.0/v6.0.0) to generate statistical analyses and visualization. Pandas (0.20.1), NumPy (v1.11.3) and SciPy (v0.19.0) were used to parse the data files and compute statistics. Holoviews (v1.8.1), Matplotlib (v2.0.2), and matplotlib-venn (v0.11.5) were used for plotting. The identification and validation of MiHAs used a Python script based on pyGeno [62] to extract MHC I peptides containing a non-synonymous polymorphic variant. The final list of MiHA was generated using Jypyter/IPython notebooks with following criteria: the peptide sequence must not be present in another protein (single genetic origin), must not be located on the chromosome Y, must not derive from HLA or IgG genes, and the minor allele frequency (MAF) must be higher or equal to 0.05 (dbSNP build 150, common). MS/MS of MiHA were manually validated (4 consecutive fragments above background required). Peak areas for MiHA peptides were extracted from PEAKS label-free quantification to compare the detection between experimental methods and cell amounts.

### Example 5: MHC I Immunopeptidome Repertoire of B-Cell Lymphoblasts Using Two Isolation Methods

The human cells selected for this study derived both from B-cells. The first model corresponds to an Epstein-Barr virus (EBV) transformed B-lymphoblastic cell line (B-LCL) obtained from normal peripheral mononuclear cells. This immortalized cell line is grown in vitro under typical cell culture conditions (see Example 4) and was described previously [26, 61, 64-66]. The second model is derived from human B acute lymphoblastic leukemia (B-ALL) cells obtained from a leukemic patient. B-ALL cells could only be expanded in vivo after injection in mice and isolation from spleen of the infected animals. High-resolution HLA genotyping was obtained for both B lymphoblastic cells and revealed two allotypes (A*02:01 and B*44:03) shared between them (Table V). As the number of MHC I peptides is proportional to the expression levels of MHC I molecules, we also determined the number of MHC I complexes localized at the cell surface for both cell type. FACS analysis (Table 1) revealed that the B-LCL cells expressed approximately 6 times more MHC I complexes (3×10⁶ molecules per cell) compared to B-ALL cells (5×10⁵ molecules per cell).

TABLE V
Description of B-lymphoblast cell models

| Cell model | Tissue origin | MHC I molecule/cell | HLA genotyping |
|---|---|---|---|
| B-LCL | B-cells EBV transformed | 3.4 × 10⁶ ± 0.72 × 10⁶ | A*02:01, A*01:01 |
| | | | B*07:02, B*44:03 |
| | | | Cw*07:02, Cw*16:01 |
| B-ALL | B-cell leukemia, mouse | 0.55 × 10⁶ ± 0.08 × 10⁶ | A*02:01, A*11:01 |
| | xenograft | | B*40:01, B*44:03 |

The work flow used for the analysis of MIPs using both MAE and IP purification methods is as follows. For the MAE approach, incubation of viable cells at low pH disrupts the MHC I complexes and releases the β2-microglobulin proteins and peptides into the buffer while membrane-bound HLA molecules remain associated with the cell surface. Peptides are desalted and then separated from the larger β2-microglobulin proteins by ultrafiltration prior to MS analyses. For the IP approach, MHC I complexes are solubilized in a detergent buffer and then captured by immuno-affinity using the W6/32 antibody coupled to a solid support. This pan-MHC I antibody recognizes the 3 HLA class I alleles (A, B and C) and is immuno-competent only for the ternary MHC I complexes when HLA molecules are associated with β2-microglobulin and peptides. The antibody/MHC I complexes are washed to remove contaminating proteins and detergent and denatured by an acidic treatment to disrupt the antibody and MCH I complexes. Peptides are then separated from the antibody, HLA molecules and β2-microglobulin by solid phase extraction prior to MS analyses on a Q-Exactive mass spectrometer. MS/MS spectra are searched with PEAKS software using protein sequence database specific to each cell type.

The reproducibility of the IP and MAE isolation methods on biological triplicates from extracts of 2, 20 and 100 million cells. Ion intensities from each LC-MS/MS data set were correlated between biological replicates. Excellent reproducibility with Pearson coefficients typically exceeding 0.9 was obtained for most cell extracts except for the MAE isolation of 2 and 20 million B-LCL cells and the IP isolation of 2 million B-ALL cells where reproducibility was lower. Next, the recovery yield of peptides identified for all experiments was examined. Peptides identified by both methods increased progressively with cell numbers for both isolation methods and cell models. For example, the number of peptides identified by the IP method increased from 2016 to 5093 peptides for 2 to 100 million B-LCL cells, respectively. On average, a 5.4-fold increase in the number of peptides identified in B-LCL cells relative to B-ALL cells, consistent with the abundance of MHC I molecules at the cell surface (Table V). The comparison of peptides identified for both cell models indicated that the IP method consistently provided more identification than the MAE method, though this difference decreased gradually with increasing cell amounts. A closer examination of these results revealed that the IP method typically provided a higher proportion of MIPs compared to MAE with enrichment levels ranging from 90 to 92% compared to 81-92% for the MAE.

In all experiments, more than 95% of all identified peptides were of length 8-15 amino acids. For B-LCL cells, the relative proportion of MIPs corresponded to approximately 80% of all peptides identified by either the MAE or IP methods. In contrast, a lower proportion of MIPs were isolated from B-ALL cells, where 70% of all peptides identified were assigned to MIPs compared to only 40% for the MAE method. While each isolation method provided different recovery yields of MIPs, the distribution of peptide affinity as defined by NetMHC 4.0 was comparable for both methods with mean affinities of 40 nM for B-LCL and B-ALL cells. Each MIP was classified according to binding motif favored by alleles identified from the HLA genotyping (Table 1). From the 6048 and 3682 MIPs identified in IP and MAE extracts of B-LCL cells, 41-42%, 32-34%, 11-13%, and 12% were presented by MHC I allelic products B*44:03, B*07:02, A:02:01 and A*01:01, respectively. Similar distribution of allelic products between MAE and IP methods was also noted for the B-ALL cells, where 29-41%, 33-34%, 15-31%, and 7-11% were presented by MHC I allelic products B*40:01, B*44:03, A*11:01, and A*02:01, respectively. Collectively, these results, indicated that the IP and MAE methods provided comparable distributions of allelic products with similar affinities, and that no significant bias in HLA binding products exist between these methods. As noted above, a total of 6050 and 2350 unique MAPs were identified in B-LCL and B-ALL cells, respectively. pyGeno was used to extract MHC I peptides containing a non-synonymous polymorphic variant, and determined that a subset of 676 and 214 peptides corresponded to putative MiHA candidates in B-LCL and B-ALL cells, respectively. These peptide variants are generally defined according to their relative occurrence in subjects bearing a given HLA allele (i.e. minor allele frequency, MAF) and their association to a well-defined genetic polymorphism [11, 14, 21]. Thus, putative MiHAs from peptides that originate from a single genetic origin, do not derive from HLA or IgG genes, and have a MAF value higher or equal to 0.05 were selected. A list of MiHAs identified is presented in Tables VI and VII for peptide variants detected in B-LCL and B-ALL cells, respectively. A comparison of the number of MiHAs identified across all experiments indicated that their detection is also scaled according to cell numbers and ranged from 8 to 18 peptides and 1 to 15 peptides for IP and MAE extracts obtained from 2×10⁶ to 1×10⁸ B-LCL cells, respectively. The enhanced identification of MiHAs observed with the IP method reflects the overall increase in the recovery of MIPs compared to the MAE method. On average, the relative proportion of MiHAs identified corresponded to approximately 0.4% of the MIP repertoire, consistent with that reported earlier for B-LCL cells [26, 67].

TABLE VI
List of MiHAs identified in B-LCL cells

| | | | | | | | SEQ ID |
|---|---|---|---|---|---|---|---|
| MiHA (No.) | Gene | SNP id | MAF | Affinity | IP/MAE | HLA | NO: |
| APKKPTGA/VDL | HMGXB3 | rs6579767 | 0.20 | 20.41 | ✓/— | B*07:02 | 348-350 |
| (82) | | | | | | | |
| ASELHTSLH/Y (83) | MDN1 | rs9294445 | 0.40 | 5.76 | ✓/✓ | A*01:01 | 351-353 |
| EEV/LKLRQQL (84) | CDK5RAP2 | rs4837768 | 0.25 | 1356.23 | ✓/— | B*44:03 | 354-356 |
| EL/IDPSNTKALY | PPID | rs9410 | 0.26 | 256.62 | ✓/✓ | A*01:01 | 357-359 |
| (85) | | | | | | | |
| EI/LDPSNTKALY | PPID | rs9410 | 0.26 | 115.03 | ✓/✓ | A*01:01 | 357-359 |
| (86) | | | | | | | |
| VPNV/EKSGAL (87) | AP3B1 | rs6453373 | 0.07 | 11 | ✓/✓ | B*07:02 | 360-362 |
| IS/PRAAAERSL (88) | SERF2/ | rs12702 | 0.21 | 13.23 | ✓/✓ | B*07:02 | 363-365 |
| | HYPK | | | | | | |
| LPSDDRGP/S/TL | SBNO2 | rs2302110 | 0.15 | 18.62 | ✓/— | B*07:02 | 366-369 |
| (89) | | | | | | | |
| LC/SEKPTVTTVY | PON2 | rs7493 | 0.28 | 67.85 | ✓/✓ | A*01:01 | 370-372 |
| (90) | | | | | | | |
| RPRAPRES/NAQAI | MKI67 | rs10082533 | 0.23 | 10 | ✓/— | B*07:02 | 373-375 |
| (91) | | | | | | | |
| H/RESPIFKQF (92) | CAPG | rs6886 | 0.41 | 55 | —/✓ | B*44:03 | 376-378 |
| TPRNTYKMTSL/V | MKI67 | rs2240 | 0.23 | 36 | ✓/— | B*07:02 | 379-381 |
| VPREYI/VRAL (94) | DCAF13 | rs3134253 | 0.25 | 4 | ✓/✓ | B*07:02 | 382-384 |
| RPRARYYI/VQV | EBI3 | rs4740 | 0.45 | 19.11 | ✓/✓ | B*07:02 | 385-387 |
| (95) | | | | | | | |
| SAFADRPS/AF (96) | CYP1B1 | rs1056827 | 0.36 | 4302.15 | ✓/✓ | B*07:02 | 388-390 |
| V/APEEARPAL (97) | DCAF15 | rs7245761 | 0.13 | 15 | ✓/✓ | B*07:02 | 391-393 |
| NLDKNTV/MGY (98) | DNAJC11 | rs12137794 | 0.06 | 10 | ✓/— | A*01:01 | 394-396 |
| SPRV/APVSPLKF | RPS6KB2 | rs13859 | 0.49 | 28.23 | ✓/✓ | B*07:02 | 397-399 |
| (99) | | | | | | | |
| SL/PRPQGLSNPST | CSF1 | rs1058885 | 0.42 | 17.86 | ✓/✓ | B*07:02 | 400-402 |
| L (100) | | | | | | | |
| SPRA/VPVSPLKF | RPS6KB2 | rs13859 | 0.49 | 44.71 | ✓/✓ | B*07:02 | 397-399 |
| (101) | | | | | | | |
| TPRPIQSSP/L (102) | WIPF1 | rs4972450 | 0.09 | 5.03 | ✓/— | B*07:02 | 403-405 |
| HPR/PQEQIAL (103) | ERAP1 | rs26653 | 0.44 | 6 | ✓/✓ | B*07:02 | 406-408 |
| YYRTNHT/I/SVM | MAN2B1 | rs1054487 | 0.45 | 34 | ✓/— | 0*07:02 | 409-412 |
| (104) | | | | | | | |
| KEMDSDQQR/T/KS | CDK5RAP2 | rs3780679 | 0.08 | 331 | ✓/— | A*01:01 | 413-416 |
| Y (105) | | | | | | | |
| M/L/VELQQKAEF | CENPF | rs3795524 | 0.07 | 76 | ✓/— | B*44:03 | 417-420 |
| (106) | | | | | | | |
| S/YGGPLRSEY | FAM178A | rs10883563 | 0.43 | 4586 | ✓/— | C*07:02 | 421-423 |
| (107) | | | | | | | |
| TEAG/AVQKQW | HEATR5B | rs62621396 | 0.13 | 101 | ✓/— | B*44:03 | 424-426 |
| (108) | | | | | | | |
| RPR/HPEDQRL | HERPUD1 | rs2217332 | 0.15 | 16 | ✓/— | B*07:02 | 427-429 |
| (109) | | | | | | | |
| LPRGMQ/KPTEFFQ | PSMB8 | rs2071543 | 0.15 | 45 | ✓/✓ | B*07:02 | 430-432 |
| SL (110) | | | | | | | |
| LARPA/VSAAL (111) | MDH2 | rs6720 | 0.48 | 11 | ✓/✓ | B*07:02 | 433-435 |
| APRES/NAQAI (112) | MKI67 | rs10082533 | 0.23 | 7 | ✓/✓ | B*07:02 | 436-438 |
| R/QPRAPRESAQAI | MKI67 | rs10764749 | 0.20 | 10 | ✓/— | B*07:02 | 439-441 |
| (113) | | | | | | | |
| RP/LRKEVKEEL | MKI67 | rs1063535 | 0.50 | 13 | —/✓ | B*07:02 | 442-444 |
| (114) | | | | | | | |
| SP/LYPRVKVDF | NADSYN1 | rs7121106 | 0.10 | 158 | ✓/✓ | B*07:02 | 445-447 |
| (115) | | | | | | | |
| IPF/LSNPRVL (116) | NLRP2 | rs10403648 | 0.16 | 72 | ✓/✓ | B*07:02 | 448-450 |
| EEVTS/T/ASEDKRK | PIKFYVE | rs999890 | 0.14 | 667 | ✓/— | B*44:03 | 451-454 |
| TY (117) | | | | | | | |
| FSEPRAI/VFY (118) | PKN1 | rs2230539 | 0.19 | 4 | ✓/— | A*01:01 | 455-457 |
| VI/TDSAELQAY | PRKDC | rs7830743 | 0.18 | 162 | ✓/✓ | A*01:01 | 458-460 |
| (119) | | | | | | | |
| LPRGMQ/KPTEF | PSMB8 | rs2071543 | 0.15 | 28 | ✓/✓ | B*07:02 | 461-463 |
| (120) | | | | | | | |
| NSEEHSAK/RY | PXK | rs56384862 | 0.29 | 10 | ✓/— | A*01:01 | 464-466 |
| (121) | | | | | | | |
| TTDKR/WTSFY | RASSF5 | rs4845112 | 0.11 | 3 | ✓/✓ | A*01:01 | 467-469 |
| (122) | | | | | | | |
| S/GEMDRRNDAW | TRAPPC12 | rs11686212 | 0.47 | 58 | ✓/— | B*44:03 | 470-472 |
| (123) | | | | | | | |
| R/CPTRKPLSL (124) | TRPT1 | rs11549690 | 0.05 | 9 | ✓/✓ | B*07:02 | 473-475 |
| YTDSSSI/VLNY | UHRF1BP1L | rs60592197 | 0.06 | 4 | ✓/— | A*01:01 | 476-478 |
| (125) | | | | | | | |
| SPGK/NERHLNAL | URB1 | rs2070378 | 0.32 | 176 | ✓/— | B*07:02 | 479-481 |
| (126) | | | | | | | |
| FT/R/IESRVSSQQT | WNK1 | rs2286007 | 0.06 | 48 | ✓/— | A*01:01 | 482-485 |
| VSY (127) | | | | | | | |
| RP/L/RAGPALLL | FUCA1 | rs2070956 | 0.14 | 11 | ✓/✓ | B*07:02 | 514-517 |
| (128) | | | | | | | |
| EEA/T/SPSQQGF | ZNF548 | rs17856896 | 0.10 | 307 | ✓/— | B*44:03 | 518-521 |
| (129) | | | | | | | |

TABLE VII
List of MiHAs identified in B-ALL cells

| | | | | | | | SEQ ID |
|---|---|---|---|---|---|---|---|
| MiHA (No.) | Gene | SNP id | MAF | Affinity | IP/MAE | HLA | NO: |
| KETDVVLKV/I | AKAP12 | rs3734797 | 0.06 | 131 | ✓/— | B*40:01 | 486-488 |
| (130) | | | | | | | |
| REEPEKI/MIL | AKAP13 | rs7179919 | 0.19 | 10 | ✓/✓ | B*40:01 | 489-491 |
| (131) | | | | | | | |
| M/L/VELQQKAEF | CENPF | rs3795524 | 0.07 | 76 | ✓/— | B*44:03 | 492-495 |
| (132) | | | | | | | |
| QEEQTR/KVAL | CEP55 | rs75139274 | 0.07 | 7 | ✓/— | B*40:01 | 496-498 |
| (133) | | | | | | | |
| ATFYGPV/IKK | CUL3 | rs3738952 | 0.14 | 12 | ✓/— | A*11:01 | 499-501 |
| (134) | | | | | | | |
| E/QETAIYKGDY | HERC3 | rs1804080 | 0.19 | 48 | ✓/— | B*44:03 | 502-504 |
| (135) | | | | | | | |
| ATSNVHM/TVKK | KANK2 | rs17616661 | 0.08 | 12 | ✓/— | A*11:01 | 505-507 |
| (136) | | | | | | | |
| EEINLQR/INI (137) | MCPH1 | rs2083914 | 0.12 | 407 | ✓/✓ | B*44:03 | 508-510 |
| QE/DLIGKKEY | MIS18BP1 | rs34101857 | 0.11 | 85 | ✓/✓ | B*44:03 | 511-513 |
| (138) | | | | | | | |

The scope of the claims should not be limited by the preferred embodiments set forth in the examples, but should be given the broadest interpretation consistent with the description as a whole.

### REFERENCES

1. Schumacher T N, Schreiber R D. Neoantigens in cancer immunotherapy. Science 2015; 348:69-74.
2. Rosenberg S A, Restifo N P. Adoptive cell transfer as personalized immunotherapy for human cancer. Science 2015; 348:62-68.
3. Sharma P, Allison J P. The future of immune checkpoint therapy. Science 2015; 348:56-61.
4. Grupp S A, Kalos M, Barrett D, Aplenc R, Porter D L, Rheingold S R et al. Chimeric antigen receptor-modified T cells for acute lymphoid leukemia. N. Engl. J Med 2013; 368:1509-1518.
5. Maus M V, Fraietta J A, Levine B L, Kalos M, Zhao Y, June C H. Adoptive immunotherapy for cancer or viruses. Annu. Rev. Immunol 2014; 32:189-225.
6. Maus M V, Grupp S A, Porter D L, June C H. Antibody modified T cells: CARs take the front seat for hematologic malignancies. Blood 2014; 123:2625-2635.
7. Reddy P, Maeda Y, Liu C, Krijanovski O I, Korngold R, Ferrara J L. A crucial role for antigen-presenting cells and alloantigen expression in graft-versus-leukemia responses. Nat. Med. 2005; 11:1244-1249.
8. Vincent K, Roy D C, Perreault C. Next-generation leukemia immunotherapy. Blood 2011; 118:2951-2959.
9. Mutis T, Brand R, Gallardo D, van B A, Niederwieser D, Goulmy E. Graft-versus-host driven graft-versus-leukemia effect of minor histocompatibility antigen HA-1 in chronic myeloid leukemia patients. Leukemia 2010; 24:1388-1392.
10. Jenq R R, van den Brink M R. Allogeneic haematopoietic stem cell transplantation: individualized stem cell and immune therapy of cancer. Nat Rev. Cancer 2010; 10:213-221.
11. Roopenian D, Choi E Y, Brown A. The immunogenomics of minor histocompatibility antigens. Immunol. Rev. 2002; 190:86-94.
12. Mullally A, Ritz J. Beyond HLA: the significance of genomic variation for allogeneic hematopoietic stem cell transplantation. Blood 2007; 109:1355-1362.
13. Granados D P, Laumont C M, Thibault P, Perreault C. The nature of self for T cells—a systems-level perspective. Curr. Opin. Immunol. 2015; 34:1-8.
14. Warren E H, Zhang X C, Li S, Fan W, Storer B E, Chien J W et al. Effect of MHC and non-MHC donor/recipient genetic disparity on the outcome of allogeneic HCT. Blood 2012; 120:2796-2806.
15. Blazer B R, Murphy W J, Abedi M. Advances in graft-versus-host disease biology and therapy. Nat Rev Immunol 2012; 12:443-458.
16. Fontaine P, Roy-Proulx G, Knafo L, Baron C, Roy D C, Perreault C. Adoptive transfer of T lymphocytes targeted to a single immunodominant minor histocompatibility antigen eradicates leukemia cells without causing graft-versus-host disease. Nat. Med. 2001; 7:789-794. 15691.137
17. Meunier M C, Delisle J S, Bergeron J, Rineau V, Baron C, Perreault C. T cells targeted against a single minor histocompatibility antigen can cure solid tumors. Nat. Med. 2005; 11:1222-1229.
18. Li N, Matte-Martone C, Zheng H, Cui W, Venkatesan S, Tan H S et al. Memory T cells from minor histocompatibility antigen-vaccinated and virus-immune donors improves GVL and immune reconstitution. Blood 2011; 118:5965-5976.
19. Blankenstein T, Leisegang M, Uckert W, Schreiber H. Targeting cancer-specific mutations by T cell receptor gene therapy. Curr. Opin. Immunol. 2015; 33:112-119.
20. Schreiber R D, Old L J, Smyth M J. Cancer immunoediting: integrating immunity's roles in cancer suppression and promotion. Science 2011; 331:1565-1570.
21. Spierings E, Hendriks M, Absi L, Canossi A, Chhaya S, Crowley J et al. Phenotype frequencies of autosomal minor histocompatibility antigens display significant differences among populations. PLoS. Genet. 2007; 3:e103.
22. Yadav M, Jhunjhunwala S, Phung Q T, Lupardus P, Tanguay J, Bumbaca S et al. Predicting immunogenic tumour mutations by combining mass spectrometry and exome sequencing. Nature 2014; 515:572-576.
23. Bleakley M, Riddell S R. Exploiting T cells specific for human minor histocompatibility antigens for therapy of leukemia. Immunol. Cell Biol. 2011; 89:396-407.
24. Hombrink P, Hassan C, Kester M G, Jahn L, Pont M J, de Ru A H et al. Identification of biological relevant minor histocompatibility antigens within the B-lymphocyte-derived HLA-ligandome using a reverse immunology approach. Clin. Cancer Res. 2015; 21:2177-2186.
25. Van Bergen C A, Rutten C E, Van Der Meijden E D, Van Luxemburg-Heijs S A, Lurvink E G, Houwing-Duistermaat J J et al. High-throughput characterization of 10 new minor histocompatibility antigens by whole genome association scanning. Cancer Res. 2010; 70:9073-9083.
26. Granados D P, Sriranganadane D, Daouda T, Zieger A, Laumont C M, Caron-Lizotte O et al. Impact of genomic polymorphism on the repertoire of human MHC class I-associated peptides. Nat Commun 2014; 5:3600.
27. Dolan B P, Sharma A A, Gibbs J S, Cunningham T J, Bennink J R, Yewdell J W. MHC class I antigen processing distinguishes endogenous antigens based on their translation from cellular vs. viral mRNA. Proc. Natl. Acad. Sci. U.S.A 2012; 109:7025-7030.
28. Yewdell J W. DRiPs solidify: progress in understanding endogenous MHC class I antigen processing. Trends Immunol. 2011; 32:548-558.
29. Nesvizhskii A I. Proteogenomics: concepts, applications and computational strategies. Nat Methods 2014; 11:1114-1125. 15691.137
30. Fagerberg L, Hallstrom B M, Oksvold P, Kampf C, Djureinovic D, Odeberg J et al. Analysis of the human tissue-specific expression by genome-wide integration of transcriptomics and antibody-based proteomics. Mol Cell Proteomics. 2014; 13:397-406.
31. Wolff M, Greenberg P D. Antigen-specific activation and cytokine-facilitated expansion of naive, human CD8⁺ T cells. Nat Protoc. 2014; 9:950-966.
32. Janelle V, Carli C, Taillefer J, Orio J, Delisle J S. Defining novel parameters for the optimal priming and expansion of minor histocompatibility antigen-specific T cells in culture. J Transl. Med. 2015; 13:123.
33. Abecasis G R, Altshuler D, Auton A, Brooks L D, Durbin R M, Gibbs R A et al. A map of human genome variation from population-scale sequencing. Nature 2010; 467:1061-1073.
34. Spierings E. Minor histocompatibility antigens: past, present, and future. Tissue Antigens 2014; 84:374-60.
35. Majewski J, Ott J. Distribution and characterization of regulatory elements in the human genome. Genome Res. 2002; 12:1827-1836.
36. Asakura S, Hashimoto D, Takashima S, Sugiyama H, Maeda Y, Akashi K et al. Alloantigen expression on non-hematopoietic cells reduces graft-versus-leukemia effects in mice. J. Clin. Invest 2010; 120:2370-2378.
37. Flutter B, Edwards N, Fallah-Arani F, Henderson S, Chai J G, Sivakumaran S et al. Nonhematopoietic antigen blocks memory programming of alloreactive CD8⁺ T cells and drives their eventual exhaustion in mouse models of bone marrow transplantation. J. Clin. Invest 2010; 120:3855-3868.
38. Fortier M H, Caron E, Hardy M P, Voisin G, Lemieux S, Perreault C et al. The MHC class I peptide repertoire is molded by the transcriptome. J. Exp. Med. 2008; 205:595-610.
39. Hoof I, van Baarle D, Hildebrand W H, Kesmir C. Proteome sampling by the HLA class I antigen processing pathway. PLoS. Comput Biol. 2012; 8:e1002517.
40. Granados D P, Yahyaoui W, Laumont C M, Daouda T, Muratore-Schroeder T L, Cote C et al. MHC I-associated peptides preferentially derive from transcripts bearing miRNA response elements. Blood 2012; 119:e181-e191.
41. Caron E, Vincent K, Fortier M H, Laverdure J P, Bramoulle A, Hardy M P et al. The MHC I immunopeptidome conveys to the cell surface an integrative view of cellular regulation. Mol. Syst. Biol. 2011; 7:533.
42. Bennett J M, Catovsky D, Daniel M T, Flandrin G, Galton D A, Gralnick H R et al. Proposed revised criteria for the classification of acute myeloid leukemia. A report of the French-American-British Cooperative Group. Ann. Intem. Med. 1985; 103:620-625.
43. Chappell P, Meziane E K, Harrison M, Magiera L, Hermann C, Mears L et al. Expression levels of MHC class I molecules are inversely correlated with promiscuity of peptide binding. Elife. 2015; 4:e05345. 15691.137
44. Schellens I M, Hoof I, Meiring H D, Spijkers S N, Poelen M C, van Gaans-van den Brink J A et al. Comprehensive analysis of the naturally processed peptide repertoire: differences between HLA-A and B in the immunopeptidome. PLoS. One. 2015; 10:e0136417.
45. Caron E, Espona L, Kowalewski D J, Schuster H, Ternette N, Alpizar A et al. An open-source computational and data resource to analyze digital maps of immunopeptidomes. Elife. 2015; 4:
46. Heath W R, Carbone F R. The skin-resident and migratory immune system in steady state and memory: innate lymphocytes, dendritic cells and T cells. Nat Immunol 2013; 14:978-985.
47. Bollard C M, Barrett A J. Cytotoxic T lymphocytes for leukemia and lymphoma. Hematology. Am. Soc. Hematol. Educ. Program. 2014; 2014:565-569.
48. Yee C. The use of endogenous T cells for adoptive transfer. Immunol Rev 2014; 257:250-263.
49. Perreault C, Roy D C, Fortin C. Immunodominant minor histocompatibility antigens: the major ones. Immunol. Today 1998; 19:69-74.
50. Choi E Y, Christianson G J, Yoshimura Y, Jung N, Sproule T J, Malarkannan S et al. Real-time T-cell profiling identifies H60 as a major minor histocompatibility antigen in murine graft-versus-host disease. Blood 2002; 100:4259-4264.
51. Kim J, Ryu S J, Oh K, Ju J M, Jeon J Y, Nam G et al. Memory programming in CD8(+) T-cell differentiation is intrinsic and is not determined by CD4 help. Nat Commun 2015; 6:7994.
52. Rosinski S L, Stone B, Graves S S, Fuller D H, De Rosa S C, Spies G A et al. Development of a minor histocompatibility antigen vaccine regimen in the canine model of hematopoietic cell transplantation. Transplantation 2015; 99:2083-2094.
53. Turtle C J, Riddell S R. Genetically retargeting CD8⁺ lymphocyte subsets for cancer immunotherapy. Curr. Opin. Immunol. 2011; 23:299-305.
54. Nauerth M, Weissbrich B, Knall R, Franz T, Dossinger G, Bet J et al. TCR-ligand koff rate correlates with the protective capacity of antigen-specific CD8+ T cells for adoptive transfer. Sci. Transl. Med. 2013; 5:192 ra87.
55. Ghosh A, Holland A M, van den Brink M R. Genetically engineered donor T cells to optimize graft-versus-tumor effects across MHC barriers. Immunol Rev 2014; 257:226-236.
56. June C H, Riddell S R, Schumacher T N. Adoptive cellular therapy: A race to the finish line. Sci. Transl. Med. 2015; 7:280ps7.
57. Torikai H, Reik A, Soldner F, Warren E H, Yuen C, Zhou Y et al. Toward eliminating HLA class I expression to generate universal cells from allogeneic donors. Blood 2013; 122:1341-1349.
58. Inaguma Y, Akahori Y, Murayama Y, Shiraishi K, Tsuzuki-lba S, Endoh A et al. Construction and molecular characterization of a T-cell receptor-like antibody and CAR-T cells specific for minor histocompatibility antigen HA-1H. Gene Ther. 2014; 21:575-584. 15691.137
59. Nielsen M, Lundegaard C, Worning P, Lauemoller S L, Lamberth K, Buus S, Brunak S, Lund O. Protein Sci., 2003 12:1007-17.
60. Andreatta M, Nielsen M. Bioinformatics 2016 Feb. 15; 32(4):511-7.
61. Lamoliatte, F., McManus, F. P., Maarifi, G., Chelbi-Alix, M. K., Thibault, P., Uncovering the SUMOylation and ubiquitylation crosstalk in human cells using sequential peptide immunopurification. Nat Commun 2017, 8, 14109.
62. Daouda, T., Perreault, C., Lemieux, S., pyGeno: A Python package for precision medicine and proteogenomics. F1000Res 2016, 5, 381.
63. Garrison, E., Gabor, M., Haplotype-based variant detection from short-read sequencing. arXiv 2012, 1207.3907.
64. Bassani-Sternberg, M., Pletscher-Frankild, S., Jensen, L. J., Mann, M., Mass spectrometry of human leukocyte antigen class I peptidomes reveals strong effects of protein abundance and turnover on antigen presentation. Mol Cell Proteomics 2015, 14, 658-673.
65. Hassan, C., Kester, M. G., de Ru, A. H., Hombrink, P., et al., The human leukocyte antigen-presented ligandome of B lymphocytes. Mol Cell Proteomics 2013, 12, 1829-1843.
66. Berlin, C., Kowalewski, D. J., Schuster, H., Mirza, N., et al., Mapping the HLA ligandome landscape of acute myeloid leukemia: a targeted approach toward peptide-based immunotherapy. Leukemia 2015, 29, 647-659.
67. Granados, D. P., Rodenbrock, A., Laverdure, J. P., Cote, C., et al., Proteogenomic-based discovery of minor histocompatibility antigens with suitable features for immunotherapy of hematologic cancers. Leukemia 2016, 30, 1344-1354.

1-47. (canceled)

48. A method of treating cancer, said method comprising administering to a subject expressing a major histocompatibility complex (MHC) class I molecules of the HLA-B*07:02 allele in need thereof an effective amount of CD8⁺ T lymphocytes recognizing a MHC class I molecule of the HLA-B*07:02 allele loaded with a minor histocompatibility antigen (MiHA) peotide of 8 to 14 amino acids comprising any one of the sequences set forth in SEQ ID NO:18, 21, 24, 27, 30, 74, 80, 123, 132, 135, 141, 144, 167, 171, 174, 183, 201, 204, 216, 225 or 228, or a combination thereof.

49-50. (canceled)

51. The method of claim 48, wherein said CD8⁺ T lymphocytes are ex vivo expanded CD8⁺ T lymphocytes.

52. The method of claim 48, wherein said method further comprises expanding said CD8⁺ T lymphocytes in the presence of cells expressing said MHC class I molecule of the HLA-B*07:02 allele loaded with said MiHA peptide in vitro prior to administration to the subject, and wherein said CD8⁺ T lymphocytes are from a second subject that does not express said MiHA peptide.

53. The method of claim 48, wherein said subject in need thereof is an allogeneic stem cell transplantation (ASCT) recipient.

54. The method of claim 48, further comprising administering an effective amount of (i) the MiHA peptide recognized by said CD8⁺ T lymphocytes, and/or (ii) a cell expressing at its surface MHC class I molecules comprising the MiHA peptide defined in (i) in their peptide binding groove.

55. The method of claim 48, wherein said cancer is a hematologic cancer.

56. The method of claim 55, wherein said hematologic cancer is a leukemia, a lymphoma or a myeloma.

57-65. (canceled)

66. The method of claim 48, wherein said MiHA peptide consists of any one of the sequences set forth in SEQ ID NO:18, 21, 24, 27, 30, 74, 80, 123, 132, 135, 141, 144, 167, 171, 174, 183, 201,204, 216, 225 or 228.

67. The method of claim 48, wherein said MiHA peptide comprises the sequence set forth in SEQ ID NO: 204.

68. The method of claim 67, wherein said MiHA peptide consists of the sequence set forth in SEQ ID NO: 204.

69. The method of claim 52, wherein said subject in need thereof is an allogeneic stem cell transplantation (ASCT) recipient.

70. The method of claim 52, further comprising administering an effective amount of (i) the MiHA peptide recognized by said CD8⁺ T lymphocytes, and/or (ii) a cell expressing at its surface MHC class I molecules comprising the MiHA peptide defined in (i) in their peptide binding groove.

71. The method of claim 52, wherein said cancer is a hematologic cancer.

72. The method of claim 71, wherein said hematologic cancer is a leukemia, a lymphoma or a myeloma.

73. The method of claim 66, wherein said subject in need thereof is an allogeneic stem cell transplantation (ASCT) recipient.

74. The method of claim 66, further comprising administering an effective amount of (i) the MiHA peptide recognized by said CD8⁺ T lymphocytes, and/or (ii) a cell expressing at its surface MHC class I molecules comprising the MiHA peptide defined in (i) in their peptide binding groove.

75. The method of claim 66, wherein said cancer is a hematologic cancer.

76. The method of claim 75, wherein said hematologic cancer is a leukemia, a lymphoma or a myeloma.
